(19) United States
(12) Patent Application Publication
(10) Pub. No.: US 2024/0352013 A1
(43) Pub. Date: Oct. 24, 2024

(54) BICYCLIC FUSED PYRAZOLE DERIVATIVES FOR THE TREATMENT OF RESPIRATORY INFECTIONS INCLUDING RSV

(71) Applicant: GEORGIA STATE UNIVERSITY RESEARCH FOUNDATION, INC., Atlanta, GA (US)

(72) Inventors: Richard Plemper, Decatur, GA (US); Julien Sourimant, Atlanta, GA (US)

(21) Appl. No.: 18/682,755

(22) PCT Filed: Jul. 29, 2022
(86) PCT No.: PCT/US22/38774

§ 371 Date: Feb. 9, 2024

Related U.S. Application Data
(60) Provisional application No. 63/232,952, filed on Aug. 13, 2021.

(51) Int. Cl. C07D 471/04; A61K 31/444; A61P 31/14

(52) U.S. Cl. CPC C07D 471/04; A61K 31/444; A61P 31/14

(57) ABSTRACT

Disclosed herein are compounds and compositions for inhibiting, treating or preventing respiratory infections, illnesses, diseases, and other respiratory conditions such as those caused by viruses including RSV, coronavirus, and related members of the pneumovirus and paramyxovirus families such as human metapneumovirus, mumps virus, human parainfluenzaviruses, and Nipah and hendra virus. Methods of inhibition, treatment or prevention of infections and diseases caused by these viruses are also provided.

### CROSS-REFERENCE TO RELATED APPLICATIONS

This application claims the benefit of U.S. Provisional Application Ser. No. 63/232,952, filed Aug. 13, 2021, the disclosure of said application being incorporated herein in its entirety.

### STATEMENT REGARDING FEDERALLY SPONSORED RESEARCH AND DEVELOPMENT

This invention was made with government support under grant numbers All 53400, A1071002, and HD079327 awarded by the National Institutes of Health. The government has certain rights in the invention.

### FIELD

The invention relates to the use of small molecule therapeutics for the treatment of respiratory infections and diseases such as respiratory syncytial virus (RSV) and coronaviruses, as well as related members of the pneumovirus and paramyxovirus family such as human metapneumovirus, mumps virus, human parainfluenzaviruses, and Nipah and hendra virus.

### BACKGROUND

Respiratory syncytial virus (RSV) is a member of the paramyxovirus family, which consists of mostly highly contagious nonsegmented, negative polarity RNA viruses that spread through the respiratory route. Specifically, RSV is a member of the order Mononegavirales, which consists of the non-segmented negative strand RNA viruses in the Families Paramyxoviridae, Pneumoviridae; Bunyaviridae, Rhabdoviridae and Filoviridae. RSV of humans (often also termed RSV or HRSV) is a member of the Pneumoviridae. Based on genetic and antigenic variations in the structural proteins, RSV is classified into two subgroups, A and B (Mufson, M. et al., J. Gen. Virol. 66:2111-2124). Other members of the Pneumovirus family include viruses such as bovine RSV (BRSV), ovine RSV (ORSV), pneumonia virus of mice (PVM), and the human metapneumoviruses amongst others.

In addition to the genome features described above, family characteristics include a lipid envelope containing one or more glycoprotein species considered to be associated with attachment and entry of the host cell. Entry is considered to require a process by which the viral envelope fuses with the membrane of the host cell. Fusion of infected cells with, for example, their neighbors, can also result in the formation of fused multinucleate cells known as syncytia in some cases. The fusion process is believed to be glycoprotein mediated and is a feature shared with diverse enveloped viruses in other taxonomic groups. In the case of the pneumo- and paramyxoviruses, virions characteristically express a fusion glycoprotein (F), which mediates membrane fusion.

Respiratory syncytial virus (RSV) is the leading cause of acute upper and lower respiratory tract infections (LRTI) in adults, young children and infants. Although at risk populations include the hospitalized, elderly and high-risk adults, RSV is primarily considered to be a pediatric disease due to the prevalence and severity of unfavorable outcomes in infants. Acute LRTI infections are a leading cause of global childhood mortality and morbidity. Serological evidence indicates that in the western world approximately 95% of all children have been infected with RSV by the age of two and 100% of children have been exposed by the time they reach adulthood.

RSV disease is thus the leading cause of virus infection-induced death among children less than 1 year of age and can be life-threatening to the elderly and the immunocompromised. Reinfection with RSV can occur throughout life, but infants born prematurely, or with bronchopulmonary dysplasia or a congenital heart defect, are at highest risk of developing severe disease. In a typical case, initial RSV infection of airway epithelia cells is followed by rapid spread from the nasopharynx to the lower airways that can affect respiratory function through excessive mucus, necrotic epithelial debris, and inflammatory cells obstructing the airways.

RSV is a seasonal infectious disease that generally runs from November to March/April in the Northern Hemisphere. In more tropical climates, the annual epidemics are more variable, often coinciding with the wet season. In most cases the RSV infections will only cause minor upper respiratory illness with symptoms resembling that of the common cold. However, severe infection with the virus may result in bronchiolitis or pneumonia, which may result in hospitalization or death. Further, since the immune response to RSV infection is not protective, RSV infections reoccur throughout adulthood. Annual re-infection rates in adults of 3-6% have been observed.

RSV infections place a significant burden on the healthcare system. This is particularly so in the case of infants such as, for example, immunodeficient infants, which on average spend twice as long in hospital as other patients with an RSV infection (7-8 days compared to 3-4 days). Hospitalization of infants with acute RSV-related bronchiolitis or RSV-related pneumonia involves supportive care management with oxygen therapy, fluids to prevent dehydration, nasal suctioning and respiratory support. There is also an economic impact associated with parents taking time away from work to care for their child.

Attempts to develop an effective RSV vaccine have been fruitless thus far, because the virus is poorly immunogenic overall and neutralizing antibody titers wane quickly after infection. Although ribavirin has been approved for RSV treatment, it has not been widely adopted in clinical use due to efficacy and toxicity issues. The humanized neutralizing antibody palivizumab is used for immunoprophylaxis of high-risk pediatric patients, but high costs prohibit broad-scale implementation.

Another more virulent form of respiratory disease is caused by coronaviruses such as coronavirus SARS-CoV-2 which is an RNA virus. Reportedly, this virus first erupted in China in November 2019. The disease caused by this virus is called COVID-19 (signifying a coronavirus disease that erupted in the year 2019) according to the World Health Organization. The official name given to this virus is SARS-CoV-2 by the International Committee on Taxonomy of Viruses. An earlier form of coronavirus caused a condition known as SARS, and SARS coronavirus was observed to cause severe respiratory illness in humans. Although the earlier form of SARS had been mostly contained since about 2004, the SARS-CoV-2 or Covid-19 virus has spread exponentially and is a significant cause of respiratory illness to millions of patients worldwide.

According to a mechanism of infection, the COVID-19 virus enters the respiratory tract and makes contact with lung cells. The S-protein (spike) of the virus contacts a host cell at a receptor site (ACE-2) on the epithelial cells of the lung. The S-protein divides into two types of SP-1 and SP-2. SP-2 helps the virus to integrate with the host cell membrane and thus the virus makes entry into the cell. Inside the human cell, the viral RNA can make its progress through several biochemical or molecular mechanisms. They are as follows: The COVID-19 viral RNA can behave like MRNA (positive sense) and make proteins through translation and also replicate to form RNA strands, or the viral RNA may make MRNA using their own and host cell enzymes (negative sense RNA virus) and thus make proteins through translation and make RNA strands through replication with the aid of RNA dependent RNA polymerase enzyme. Sometimes viral RNA may be converted to DNA through the aid of reverse transcriptase enzyme. This new DNA copy will integrate with the host DNA and thus transcribe the MRNA to make proteins and copies of viral RNA. No matter which way the RNA and proteins are replicated and translated, they will be assembled into active virus particles in the endothelial reticulum attached to the Golgi bodies of the host cells. The new virus then attacks neighboring cells, thus continuing their destruction of the lung tissue.

The COVID-19 virus SARS-CoV-2 preferentially attacks the ciliated cells of the upper respiratory tract and also the type-II pneumocytes in the alveoli, thus causing inflammation and pneumonia. Although there are now preventative vaccines against SARS-CoV-2, it still results in millions of cases around the world, and treatment options remain limited.

One previous attempt at developing successful compounds was disclosed in U.S. Pat. No. 10,906,899, said patent incorporated herein by reference. However, in light of the prevalence of viruses that can cause respiratory disease and their ability to evolve different strains that may have more severe effects and higher levels of contagion, there is a continuing need to develop additional compounds that are effective against RSV and other viruses that cause respiratory disease.

Accordingly, there remains an urgent and unmet need for new compounds that are useful in the treatment and prevention of respiratory diseases and infections from viruses such as RSV and coronavirus. Small-molecule drug-like therapeutics have high promise to provide a novel avenue towards respiratory infectious disease management and prevention. It is therefore an object of the present invention to provide new small-molecule therapeutics classes for the treatment of human patients and other hosts infected with viruses that attach the respiratory system such as RSV and coronaviruses.

### SUMMARY

In accordance with the invention, disclosed herein are compounds, compositions, and methods of inhibiting respiratory infections and diseases such as RSV, coronavirus, influenza, etc. or treating or preventing respiratory infection, disease, or other respiratory condition in a patient in need thereof.

In addition, the present inventors have provided compounds, compositions, and methods of inhibiting and impairing RNA elongation of synthetic primer/template RNA pairs of a virus that cause a respiratory infection, disease, or other respiratory condition in a patient.

In addition, the present inventors have provided compounds, compositions, and methods of blocking viral RNA-dependent RNA polymerase (also known as RdRP or RNA replicase), wherein the blockage can be non-competitive, said methods directed to administration to a human or animal patient in need thereof an effective amount of a compound or composition in accordance with the claimed invention.

In the description below, it will be noted that although described in particular for RSV, such methods can be applied in the same manner on viruses other than RSV such as coronavirus, influenza, and others as listed above which cause respiratory infection and disease.

In accordance with the invention, RSV can be inhibited, and RSV infection can be treated or prevented, by administering to a patient in need thereof a composition containing an anti-RSV compound of Formula 1 as indicated below:

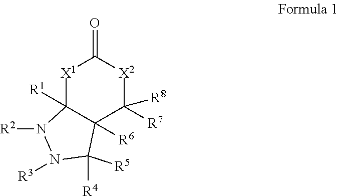

or a pharmaceutically acceptable salt thereof, wherein
X¹ and X² are independently selected from —NR⁰ and —CR^{a}R^{b};
R¹, R⁴, R⁵, R⁶, R⁷, R⁸, R^{a} and R^{b} are independently selected from —R^{c}, —OR^{c}, —N(R^{c})₂, —SR^{c}, —SO₂R^{c}, —SO₂N(R^{c})₂; —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —CF₃, —NO₂;
R⁰, R², and R³ are independently selected from —R^{c}, —SO₂R^{c}, —SO₂N(R^{c})₂; —C(O)R^{c}, —COOR^{c}, —C(O)N(R^{c})₂;
wherein two or more of R⁰, R¹, R², R³, R⁴, R⁵, R⁶, R⁷, R⁸, R^{a} and R^{b} can together form a ring;
wherein any two of the aforementioned R groups, when adjacent, can together form a double bond;
wherein two of the aforementioned R groups, when germinal, can together form a carbonyl, olefin or imine;
wherein R^{c} is in each case independently selected from hydrogen, C₁₋₈ alkyl, C₃₋₈ cycloalkyl, C₂₋₈ heterocyclyl, C₆₋₁₂ aryl, C₃₋₁₂ heteroaryl, C₁₋₈ alkyl-C₃₋₈ cycloalkyl, C₁₋₈ alkyl-C₂₋₈ heterocyclyl, C₁₋₈ alkyl-C₆₋₁₂ aryl, and C₁₋₈ alkyl-C₃₋₁₂ heteroaryl; wherein one or more of R^{c} can together form a ring with any one or more of R⁰, R¹, R², R³, R⁴, R⁵, R⁶, R⁷, R⁸, R^{a}, R^{b} and R^{c}.

More specifically, RSV can also be inhibited, and RSV infection can also be treated or prevented by administering to a patient in need thereof a composition containing an anti-RSV compound of Formula 1a as follows:

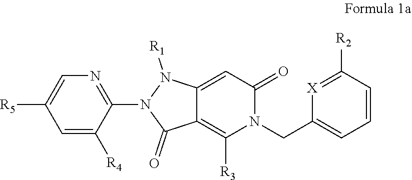

or a pharmaceutically acceptable salt thereof, wherein
X is N, C, —NR⁰, or —CR^{a}R^{b};
R⁰ and R¹ are independently selected from the group consisting of —R^{c}, —SO₂R^{c}, —SO₂N(R^{c})₂; —CR^{c}, —C(O)R^{c}, —COOR^{c}, or —C(O)N(R^{c})₂,
wherein R^{c} is selected from the group consisting of hydrogen, C₁₋₈ alkyl, C₃₋₈ cycloalkyl, C₂₋₈ heterocyclyl, C₆₋₁₂ aryl, C₃₋₁₂ heteroaryl, C₁₋₈ alkyl-C₃₋₈ cycloalkyl, C₁₋₈ alkyl-C₂₋₈ heterocyclyl, C₁₋₈ alkyl-C₆₋₁₂ aryl, and C₁₋₈ alkyl-C₃₋₁₂ heteroaryl; and wherein when R^{c} is not hydrogen, R^{c} may be optionally substituted at any position;
R², R³, R⁴, R⁵, R^{a}, and R^{b} are independently selected from the group consisting of R^{c}, —OR^{c}, —N(R^{c})₂, —SR^{c}, —SO₂R^{c}, —SO₂N(R^{c})₂; —CR^{c}, —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —CF₃, or —NO₂;
wherein two or more of R⁰, R¹, R², R³, R⁴, R⁵, R^{a} and R^{b} can together form a ring; wherein any two of the aforementioned R groups when adjacent can together form a double bond;
wherein two of the aforementioned R groups, when germinal, can together form a carbonyl, olefin or imine; and
wherein one or more of R^{c} can together form a ring with any one or more of R⁰, R¹, R², R³, R⁴, R⁵, R^{a}, R^{b} and R^{c}.

Still other compounds and compositions in accordance with the invention are provided as set forth in more detail hereinbelow.

The details of one or more embodiments are set forth in the descriptions below. Other features, objects, and advantages will be apparent from the description and from the claims.

### BRIEF DESCRIPTION OF THE DRAWING FIGURES

FIG. 1 provides a graphic representation of lung tissue tests in mice showing the anti-viral effectiveness of compounds in accordance with the invention.

FIGS. 2A-2J provide various representations of the resistance and mechanistic profiling of compound AVG-233 in accordance with the invention.

FIGS. 3A-3H provide various representations showing the label-free positive target identification of compound AVG-233 in accordance with the invention.

FIGS. 4A-4G provide various representations showing compound AVG-233 target site mapping through photo-affinity labeling.

FIGS. 5A-5H provide various representations showing the efficacy of AVG-233 in well-differentiated human airway epithelial (3D-HAE) cells grown at air-liquid interface.

FIGS. 6A-6G provide various representations showing the identification of orally efficacious developmental analogs of AVG-233.

FIG. 7 provides a graphic representation of a dose-response inhibition of RSV minireplicon in presence of resistance mutation candidates.

FIG. 8 provides a graphic representation of multi-step growth curves of recRSV-fireSMASh harboring individual resistance mutations L1502Q, Y1631H, or H1632Q.

FIG. 9 provides a graphic representation of a purified recombinant RSV RdRP (P-L) with resistance mutations or mutation N812A eliminating polymerase activity (59). Coomassie blue staining after SDS-PAGE fractionation; material representing L and P polypeptides is highlighted.

FIG. 10 provides an autoradiogram of the primer extension assay from FIG. 21.

FIG. 11 provides a side-by side comparison of AVG-233 and AZ-27 in de novo RNA synthesis assay using L preparations harboring distinct resistance mutations.

Color-coding of L preparations as in FIG. 2D-E.

FIG. 12 provides a graphic representation of an In vitro RdRP assay performed as in FIG. 1I, using the alternative primer/template pair shown.

FIG. 13 provides a graphic representation showing the effect of endogenous nucleotides on AVG-233 RdRP inhibition.

FIG. 14 provides a depiction of the immunostaining of 3D-HAE.

FIG. 15 provides a depiction of the immunolabelling of 3D-HAE.

FIG. 16 provides additional depictions of the immunostaining of 3D-HAE.

FIG. 17 provides additional depictions of the immunostaining of 3D-HAE.

FIG. 18 provides additional depictions of the immunostaining of 3D-HAE.

FIG. 19 provides a depiction of ciliated cells from 3D-HAE infected with recRSV-fireSMASh.

FIG. 20 provides a depiction of the treatment with AVG-233 of 3D-HAEs infected with recRSV-fireSMASh.

FIG. 21 provides a graphic representation of the dose-response inhibition of recRSV-fireSMASh by analogs of AVG-233 in undifferentiated primary human airway epithelial cells.

FIG. 22 provides a graphic representation of the dose-response inhibition of in vitro RdRP primer extension by analogs of AVG-233.

FIG. 23 provides a graphic representation of a side-by-side comparison of AVG-233 and AVG-388 dose-response inhibition of either RSV minireplicon (top) and recRSV-fireSMASh (bottom).

FIG. 24 provides a graphic representation of a comparison of AVG-233 and AVG-388 cytotoxicity.

FIG. 25 provides a graphic representation of mouse bodyweight and temperature as referred to in the examples.

FIG. 26 provides a depiction of lung histopathology of cells as referred to in the examples.

FIG. 27 is a schematic of the chemical synthesis strategy of the AVG scaffold.

FIG. 28 is a schematic of the chemical synthesis strategy of AVG analog C.

### DETAILED DESCRIPTION OF EXEMPLARY EMBODIMENTS

Before the present compounds, compositions, methods and systems are disclosed and described, it is to be understood that the methods and systems are not limited to specific synthetic methods, specific compounds, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes, from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

The term “alkyl” as used herein is a branched or unbranched hydrocarbon group such as methyl, ethyl, n-propyl, isopropyl, n-butyl, isobutyl, t-butyl, pentyl, hexyl, heptyl, octyl, nonyl, decyl, dodecyl, and the like. The alkyl group can also be substituted or unsubstituted. Unless stated otherwise, the term “alkyl” contemplates both substituted and unsubstituted alkyl groups. The alkyl group can be substituted with one or more groups including, but not limited to, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol as described herein. An alkyl group which contains no double or triple carbon-carbon bonds is designated a saturated alkyl group, whereas an alkyl group having one or more such bonds is designated an unsaturated alkyl group.

Unsaturated alkyl groups having a double bond can be designated alkenyl groups, and unsaturated alkyl groups having a triple bond can be designated alkynyl groups. Unless specified to the contrary, the term alkyl embraces both saturated and unsaturated groups.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is replaced with a heteroatom such as, but not limited to, nitrogen, oxygen, sulfur, selenium or phosphorus. The cycloalkyl group and heterocycloalkyl group can be substituted or unsubstituted. Unless stated otherwise, the terms “cycloalkyl” and “heterocycloalkyl” contemplate both substituted and unsubstituted cycloalkyl and heterocycloalkyl groups. The cycloalkyl group and heterocycloalkyl group can be substituted with one or more groups including, but not limited to, alkyl, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol as described herein. A cycloalkyl group which contains no double or triple carbon-carbon bonds is designated a saturated cycloalkyl group, whereas an cycloalkyl group having one or more such bonds (yet is still not aromatic) is designated an unsaturated cycloalkyl group. Unless specified to the contrary, the term alkyl embraces both saturated and unsaturated groups.

The term “aryl” as used herein is an aromatic ring composed of carbon atoms. Examples of aryl groups include, but are not limited to, phenyl and naphthyl, etc. The term “heteroaryl” is an aryl group as defined above where at least one of the carbon atoms of the ring is replaced with a heteroatom such as, but not limited to, nitrogen, oxygen, sulfur, selenium or phosphorus. The aryl group and heteroaryl group can be substituted or unsubstituted. Unless stated otherwise, the terms “aryl” and “heteroaryl” contemplate both substituted and unsubstituted aryl and heteroaryl groups. The aryl group and heteroaryl group can be substituted with one or more groups including, but not limited to, alkyl, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol as described herein.

Exemplary heteroaryl and heterocyclyl rings include: benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyL cirrnolinyl, decahydroquinolinyl, 2H,6H˜1,5,2-dithiazinyl, dihydrofuro[2,3 b]tetrahydrofuran, furanyl, furazanyl, imidazolidinyl, imidazolinyl, imidazolyl, IH-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, and xanthenyl.

The terms “alkoxy,” “cycloalkoxy,” “heterocycloalkoxy,” “cycloalkoxy,” “aryloxy,” and “heteroaryloxy” have the aforementioned meanings for alkyl, cycloalkyl, heterocycloalkyl, aryl and heteroaryl, further providing said group is connected via an oxygen atom.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. Unless specifically stated, a substituent that is said to be “substituted” is meant that the substituent is substituted with one or more of the following: alkyl, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol as described herein. In a specific example, groups that are said to be substituted are substituted with a protic group, which is a group that can be protonated or deprotonated, depending on the pH.

Unless specified otherwise, the term “patient” refers to any mammalian organism, including but not limited to, humans.

Pharmaceutically acceptable salts are salts that retain the desired biological activity of the parent compound and do not impart undesirable toxicological effects. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. Pharmaceutically acceptable and non-pharmaceutically acceptable salts may be prepared using procedures well known in the art, for example, by reacting a sufficiently basic compound such as an amine with a suitable acid comprising a physiologically acceptable anion. Alkali metal (for example, sodium, potassium, or lithium) or alkaline earth metal (for example, calcium) salts of carboxylic acids can also be made.

Disclosed herein are compounds, compositions and methods of inhibiting RSV or treating or preventing RSV infection in a patient in need thereof by administering to the patient an effective amount of at least one RSV inhibiting compound. In certain embodiments, the RSV inhibiting compound has the structure of Formula I below:

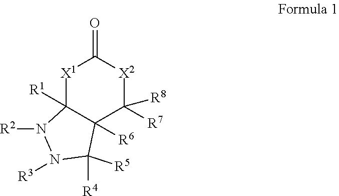

or a pharmaceutically acceptable salt thereof, wherein
X¹ and X² are independently selected from —NR⁰ and —CR^{a}R^{b};
R¹, R⁴, R⁵, R⁶, R⁷, R⁸, R^{a} and R^{b} are independently selected from —R^{c}, —OR^{c}, —N(R^{c})₂, —SR^{c}, —SO₂R^{c}, —SO₂N(R^{c})₂; —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —CF₃, —NO₂;
R⁰, R², and R³ are independently selected from —R^{c}, —SO₂R^{c}, —SO₂N(R^{c})₂; —C(O)R^{c}, —COOR^{c}, —C(O)N(R^{c})₂;
wherein two or more of R⁰, R¹, R², R³, R⁴, R⁵, R⁶, R⁷, R⁸, R^{a} and R^{b} can together form a ring;
wherein any two of the aforementioned R groups, when adjacent, can together form a double bond;
wherein two of the aforementioned R groups, when germinal, can together form a carbonyl, olefin or imine;
wherein R^{c} is in each case independently selected from hydrogen, C₁₋₈ alkyl, C₃₋₈ cycloalkyl, C₂₋₈ heterocyclyl, C₆₋₁₂ aryl, C₃₋₁₂ heteroaryl, C₁₋₈ alkyl-C₃₋₈ cycloalkyl, C₁₋₈ alkyl-C₂₋₈ heterocyclyl, C₁₋₈ alkyl-C₆₋₁₂ aryl, and C₁₋₈ alkyl-C₃₋₁₂ heteroaryl; wherein one or more of R^{c} can together form a ring with any one or more of R⁰, R¹, R², R³, R⁴, R⁵, R⁶, R⁷, R⁸, R^{a}, R^{b} and R^{c}.

More specifically, RSV can also be inhibited, and RSV infection can also be treated or prevented by administering to a patient in need thereof a composition containing an anti-RSV compound of Formula 1a as follows:

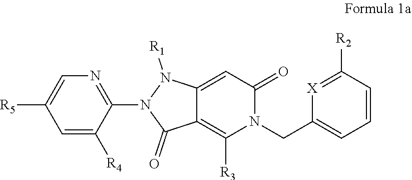

or a pharmaceutically acceptable salt thereof, wherein
X is N, C, —NR⁰, or —CR^{a}R^{b};
R⁰ and R¹ are independently selected from the group consisting of —R^{c}, —SO₂R^{c}, —SO₂N(R^{c})₂; —CR^{c}, —C(O)R^{c}, —COOR^{c}, substituted or unsubstituted benzyl, and —C(O)N(R^{c})₂,
wherein R^{c} is selected from the group consisting of hydrogen, C₁₋₈ alkyl, C₃₋₈ cycloalkyl, C₂₋₈ heterocyclyl, C₆₋₁₂ aryl, C₃₋₁₂ heteroaryl, C₁₋₈ alkyl-C₃₋₈ cycloalkyl, C₁₋₈ alkyl-C₂₋₈ heterocyclyl, C₁₋₈ alkyl-C₆₋₁₂ aryl, and C₁₋₈ alkyl-C₃₋₁₂ heteroaryl; and wherein when R^{c} is not hydrogen, R^{c} may be optionally substituted at any position;
R², R³, R⁴, R⁵, R^{a}, and R^{b} are independently selected from the group consisting of R^{c}, —OR^{c}, —N(R^{c})₂, —SR^{c}, —SO₂R^{c}, —SO₂N(R^{c})₂; —CR^{c}, —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —CF₃, or —NO₂;
wherein two or more of R⁰, R¹, R², R³, R⁴, R⁵, R^{a} and R^{b} can together form a ring;
wherein any two of the aforementioned R groups when adjacent can together form a double bond;
wherein two of the aforementioned R groups, when germinal, can together form a carbonyl, olefin or imine; and
wherein one or more of R^{c} can together form a ring with any one or more of R⁰, R¹, R², R³, R⁴, R⁵, R^{a}, R^{b} and R^{c}.

In particular embodiment, Formula 1a can have various substituents as identified above, including individual cases such as (1) wherein X is N, (2) wherein R¹ is optionally substituted C₁₋₈ alkyl-C₆₋₁₂ aryl, (3) wherein R¹ is an optionally substituted benzyl, (4) wherein R² is —CF₃, or —Cl, (5) wherein R³ is C₁₋₈ alkyl, (6) wherein R³ is methyl, and (7) wherein R⁴ and R⁵ are independently H or F. These substituents can also be used in conjunction with all of the exemplary formulas described herein, such as formulas 1b and 1 c below.

In certain embodiments of Formula 1a, R¹ can be substituted or unsubstituted benzyl, including:

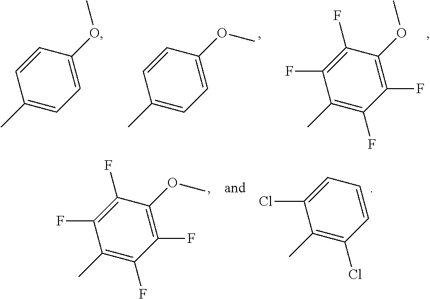

More specifically, RSV can also be inhibited, and RSV infection can also be treated or prevented by administering to a patient in need thereof a composition containing an anti-RSV compound of Formula 1b as follows:

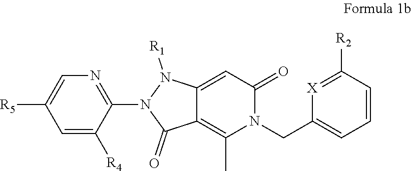

or a pharmaceutically acceptable salt thereof, wherein
X is N, C, —NR⁰, or —CR^{a}R^{b};
R⁰ and R¹ are independently selected from the group consisting of —R^{c}, —SO₂R^{c}, —SO₂N(R^{c})₂; —CR^{c}, —C(O)R^{c}, —COOR^{c}, substituted or unsubstituted benzyl, and —C(O)N(R^{c})₂,
wherein R^{c} is selected from the group consisting of hydrogen, C₁₋₈ alkyl, C₃₋₈ cycloalkyl, C₂₋₈ heterocyclyl, C₆₋₁₂ aryl, C₃₋₁₂ heteroaryl, C₁₋₈ alkyl-C₃₋₈ cycloalkyl, C₁₋₈ alkyl-C₂₋₈ heterocyclyl, C₁₋₈ alkyl-C₆₋₁₂ aryl, and C₁₋₈ alkyl-C₃₋₁₂ heteroaryl; and wherein when R^{c} is not hydrogen, R^{c} may be optionally substituted at any position;
R², R⁴, R⁵, R^{a}, and R^{b} are independently selected from the group consisting of R^{c}, —OR^{c}, —N(R^{c})₂, —SR^{c}, —SO₂R^{c}, —SO₂N(R^{c})₂; —CR^{c}, —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —CF₃, or —NO₂;
wherein two or more of R⁰, R¹, R², R⁴, R⁵, R^{a} and R^{b} can together form a ring;
wherein any two of the aforementioned R groups when adjacent can together form a double bond;
wherein two of the aforementioned R groups, when germinal, can together form a carbonyl, olefin or imine; and
wherein one or more of R^{c} can together form a ring with any one or more of R⁰, R¹, R², R⁴, R⁵, R^{a}, R^{b} and R^{c}.

Even more specifically, RSV can also be inhibited, and RSV infection can also be treated or prevented by administering to a patient in need thereof a composition containing an anti-RSV compound of Formula 1c as follows:

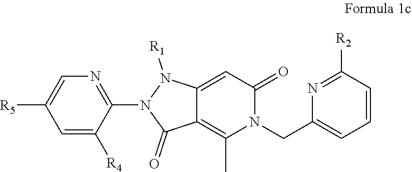

or a pharmaceutically acceptable salt thereof, wherein
R⁰ and R¹ are independently selected from the group consisting of —R^{c}, —SO₂R^{c}, —SO₂N(R^{c})₂; —CR^{c}, —C(O)R^{c}, —COOR^{c}, substituted or unsubstituted benzyl, and —C(O)N(R^{c})₂,
wherein R^{c} is selected from the group consisting of hydrogen, C₁₋₈ alkyl, C₃₋₈ cycloalkyl, C₂₋₈ heterocyclyl, C₆₋₁₂ aryl, C₃₋₁₂ heteroaryl, C₁₋₈ alkyl-C₃₋₈ cycloalkyl, C₁₋₈ alkyl-C₂₋₈ heterocyclyl, C₁₋₈ alkyl-C₆₋₁₂ aryl, and C₁₋₈ alkyl-C₃₋₁₂ heteroaryl; and wherein when R^{c} is not hydrogen, R^{c} may be optionally substituted at any position;
R², R⁴, R⁵, R^{a}, and R^{b} are independently selected from the group consisting of R^{c}, —OR^{c}, —N(R^{c})₂, —SR^{c}, —SO₂R^{c}, —SO₂N(R^{c})₂; —CR^{c}, —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —CF₃, or —NO₂;
wherein two or more of R⁰, R¹, R², R⁴, R⁵, R^{a} and R^{b} can together form a ring;
wherein any two of the aforementioned R groups when adjacent can together form a double bond;
wherein two of the aforementioned R groups, when germinal, can together form a carbonyl, olefin or imine; and
wherein one or more of R^{c} can together form a ring with any one or more of R⁰, R¹, R², R⁴, R⁵, R^{a}, R^{b} and R^{c}.

As used herein, the term olefin includes unsubstituted methylene (e.g., =CH2), as well as substituted groups including the functional groups falling with the definitions of the above R groups. The term imine includes the primary imine (e.g., =NH) as well as substituted imines including the functional groups falling with the definitions of R⁰, R², and R³.

Any of the heteroaryl groups may be substituted one or more times by —F, —Cl, —Br, —I, —CN, —NO₂, OH, COOH, C₁₋₈ alkyl, C₃₋₈ cycloalkyl, C₂₋₈ heterocyclyl, C₆₋₁₂ aryl, C₃₋₁₂ heteroaryl, C₁₋₈ alkyl-C₃₋₈ cycloalkyl, C₁₋₈ alkyl-C₂₋₃ heterocyclyl, C₁₋₈ alkyl-C₆₋₁₂ aryl, and C₁₋₈ alkyl-C₃₋₁₂ heteroaryl, C₁₋₈ alkyl-C₁₋₈ alkoxy, C₁₋₈ alkyl-C₃₋₈ cycloalkoxy, C₁₋₈ alkyl-C₂₋₃ heterocycloalkoxy, C₁₋₈ alkyl-C₆₋₁₂ aryloxy, and C₁₋₈ alkyl-C₃₋₁₂ heteroaryloxy.

The substitution may occur at any atomic position permitted by valency. Any of the above mentioned groups may be unsubstituted or substituted one or more times by —F, —Cl, —Br, —I, —CN, —NO₂, OH, COOH

In some embodiments of the above compounds, the heteroaryl group can be selected from the following:

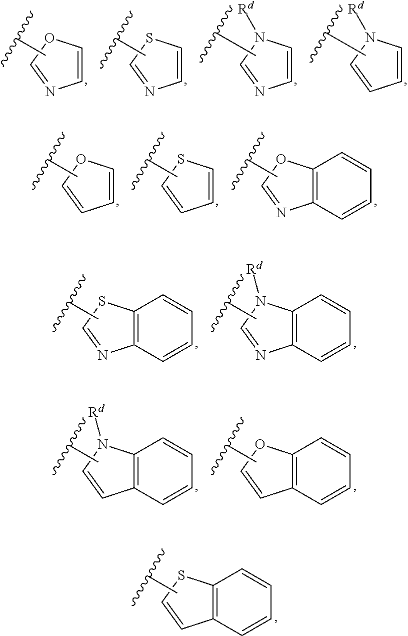

wherein R^{d} is hydrogen, C₁₋₆alkyl or a point of attachment to the compound of Formula 1. Any of the above heteroaryl groups may be attached via any atom permitted by the rules of valency. For instance, when R³ is an oxazole, thiazole, or imidazole ring, it may be connected at the 2, 4 or 5 position, as well as the 1 position in the case of imidazole. When R³ is pyrrole, furan, or thiophene ring, it may be connected at the 2, 3, 4, or 5 position, as well as the 1 position in the case of pyrrole. When R³ is benzoxazole, benzthioazole, or benzimidazole, it may be connected at the 2, 4, 5, 6, or 7 position, as well as the 1 position in the case of benzimidazole. When R³ is indole, benzofuran, or benzothiophene, it may be connected at the 2, 4, 3, 5, 6, or 7 position, as well as the 1 position in the case of indole.

Any of the heteroaryl groups may be substituted one or more times by —F, —Cl, —Br, —I, —CN, —NO₂, C₁₋₈ alkyl, C₃₋₈ cycloalkyl, C₂₋₈ heterocyclyl, C₆₋₁₂ aryl, C₃₋₁₂ heteroaryl, C₁₋₈alkyl-C₃₋₈ cycloalkyl, C₁₋₈alkyl-C₂₋₃ heterocyclyl, C₁₋₈alkyl-C₆₋₁₂ aryl, and C₁₋₈alkyl-C₃₋₁₂ heteroaryl, C₁₋₈alkyl-C₁₋₈ alkoxy, C₁₋₈alkyl-C₃₋₈ cycloalkoxy, C₁₋₈alkyl-C₂₋₈ heterocycloalkoxy, C₁₋₈alkyl-C₆₋₁₂ aryloxy, and C₁₋₈alkyl-C₃₋₁₂ heteroaryloxy. The substitution may occur at any atomic position permitted by valency.

Any of the heteroaryl groups may be substituted one or more times by —F, —Cl, —Br, —I, —CN, —NO₂, C₁₋₈ alkyl, C₃₋₈ cycloalkyl, C₂₋₈ heterocyclyl, C₆₋₁₂ aryl, C₃₋₁₂ heteroaryl, C₁₋₈alkyl-C₃₋₈ cycloalkyl, C₁₋₈alkyl-C₂₋₃ heterocyclyl, C₁₋₈alkyl-C₆₋₁₂ aryl, and C₁₋₈alkyl-C₃₋₁₂ heteroaryl, C₁₋₈alkyl-C₁₋₈ alkoxy, C₁₋₈alkyl-C₃₋₈ cycloalkoxy, C₁₋₈alkyl-C₂₋₈ heterocycloalkoxy, C₁₋₈alkyl-C₆₋₁₂ aryloxy, and C₁₋₈alkyl-C₃₋₁₂ heteroaryloxy. The substitution may occur at any atomic position permitted by valency. In preferred embodiments, the heteroaryl is selected from the group consisting of:

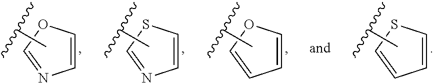

Exemplary specific compounds in accordance with the present invention are shown in Table 1 below:

TABLE 1
Compounds of the Invention

| Compound | |
|---|---|
| Designation | Structure |
| AVG-233 | 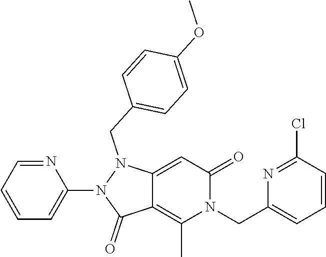 [SMILES: COc1ccc(Cn2c3cc(=O)n(Cc4cccc(Cl)n4)c(C)c3c(=O)n2-c2ccccn2)cc1] |
| AVG-388 | 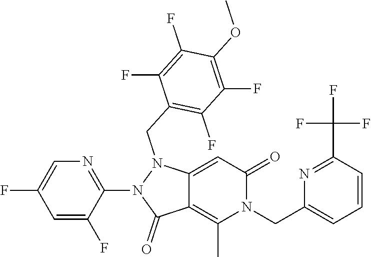 [SMILES: COc1c(F)c(F)c(Cn2c3cc(=O)n(Cc4cccc(C(F)(F)F)n4)c(C)c3c(=O)n2-c2ncc(F)cc2F)c(F)c1F] |
| AVG-389 | 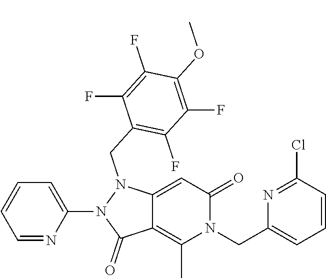 [SMILES: COc1c(F)c(F)c(Cn2c3cc(=O)n(Cc4cccc(Cl)n4)c(C)c3c(=O)n2-c2ccccn2)c(F)c1F] |
| AVG-390 | 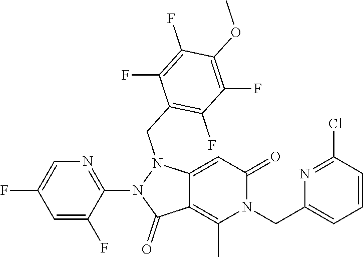 [SMILES: COc1c(F)c(F)c(Cn2c3cc(=O)n(Cc4cccc(Cl)n4)c(C)c3c(=O)n2-c2ncc(F)cc2F)c(F)c1F] |
| AVG-391 | 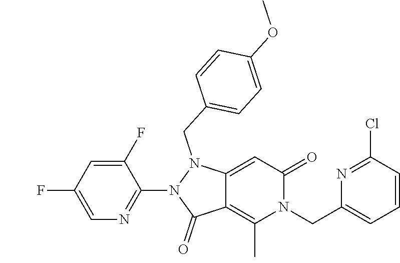 [SMILES: COc1ccc(Cn2c3cc(=O)n(Cc4cccc(Cl)n4)c(C)c3c(=O)n2-c2ncc(F)cc2F)cc1] |
| AVG-392 | 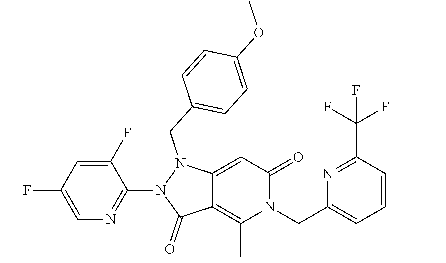 [SMILES: COc1ccc(Cn2c3cc(=O)n(Cc4cccc(C(F)(F)F)n4)c(C)c3c(=O)n2-c2ncc(F)cc2F)cc1] |
| AVG-435 | 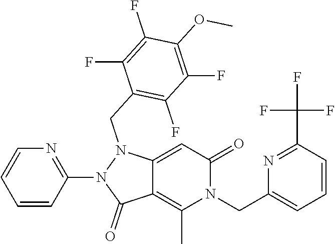 [SMILES: COc1c(F)c(F)c(Cn2c3cc(=O)n(Cc4cccc(C(F)(F)F)n4)c(C)c3c(=O)n2-c2ccccn2)c(F)c1F] |
| AVG-436 | 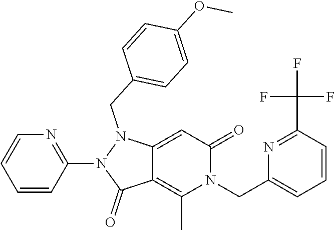 [SMILES: COc1ccc(Cn2c3cc(=O)n(Cc4cccc(C(F)(F)F)n4)c(C)c3c(=O)n2-c2ccccn2)cc1] |
| AVG-445 | 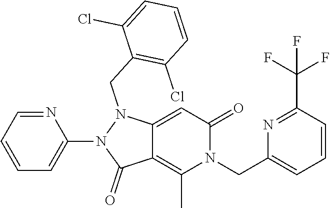 [SMILES: Cc1c2c(=O)n(-c3ccccn3)n(Cc3c(Cl)cccc3Cl)c2cc(=O)n1Cc1cccc(C(F)(F)F)n1] |
| AVG-446 | 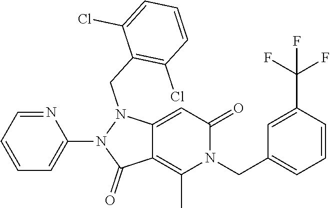 [SMILES: Cc1c2c(=O)n(-c3ccccn3)n(Cc3c(Cl)cccc3Cl)c2cc(=O)n1Cc1cccc(C(F)(F)F)c1] |

The compounds defined in the above aspects are RSV antiviral agents and are useful in the treatment of RSV infections. Accordingly, these compounds of the invention are useful in the treatment of RSV disease, such as bronchiolitis or pneumonia, or in reducing exacerbation of underlying or pre-existing respiratory diseases or conditions wherein RSV infection is a cause of said exacerbation. The underlying or pre-existing respiratory diseases or conditions may include asthma, chronic obstructive pulmonary disease (COPD) and immunosuppression such as immunosuppression experienced by bone marrow transplant recipients. The compounds above may also be combined with one or more other RSV antiviral agents.

The compounds of the invention may be formulated as pharmaceutical compositions and administered to a human patient as set forth in more detail below. The compounds can be delivered in a number of suitable ways including orally, intravenously, topically, parentally, subcutaneously, intradermally, or by inhalation. Exemplary routes of administration include buccal, oral, intravenous, intramuscular, topical, subcutaneous, rectal, vaginal, parenteral, pulmonary, intranasal, ophthalmic, and the like, as set forth in more detail below.

Useful dosages of the compounds of the invention for inclusion in the pharmaceutical compositions of the invention can be determined by comparing in vitro activity and in vivo activity of the compounds in appropriate animal models. Generally, the concentration of the compound(s) of the invention in a liquid composition will range from about 0.1% to about 95% by weight, preferably from about 0.5% to about 25% by weight. The concentration in a semi-solid or solid composition will range from about 0.1% to 100% by weight, preferably about 0.5% to about 5% by weight. Single doses for intravenous injection, subcutaneous, intramuscular or topical administration, infusion, ingestion or suppository will generally be from about 0.001 to about 5000 mg, and be administered from about 1 to about 3 times daily, to yield levels of about 0.01 to about 500 mg/kg, for adults.

The compounds can be co-administered with one or more other agents for the treatment or prevention of RSV infection. The other agents can be formulated separately, and administered either at the same or different time as the compounds of the instant invention. The other agents can be co-formulated with the compounds of the instant invention to give a combination dosage form.

### Pharmaceutical Compositions and Modes of Administration

The invention also provides a pharmaceutical composition comprising a compound of the formulas as described above and a pharmaceutically acceptable vehicle, excipient or carrier, and the form of this composition can be suitable for a number of different modes of administration to a patient as set forth below.

The pharmaceutical composition may further comprise or be administered in combination with one or more other RSV antiviral agents such as Virazole®, BMS-4337715, TMC3531216, MDT-637 (formerly VP-14637), GS-5806, RSV604, ALNRSV01, AL-8176 (or ALS-8176) and/or other agents that may be developed as inhibitors of viral entry, assembly, replication, egress or host-virus interactions

The term “composition” is intended to include the formulation of an active ingredient with conventional vehicles, carriers and excipients, and also with encapsulating materials as the carrier, to give a capsule in which the active ingredient (with or without other carriers) is surrounded by the encapsulation carrier. Any carrier must be “pharmaceutically acceptable” meaning that it is compatible with the other ingredients of the composition and is not deleterious to a subject. The compositions of the present invention may contain other therapeutic agents as described above, and may be formulated, for example, by employing conventional solid or liquid vehicles or diluents, as well as pharmaceutical additives of a type appropriate to the mode of desired administration (for example, excipients, binders, preservatives, stabilizers, flavours and the like) according to techniques such as those well known in the art of pharmaceutical formulation (see, for example, Remington: The Science and Practice of Pharmacy, 21st Ed., 2005, Lippincott Williams & Wilkins).

The pharmaceutical composition includes those suitable for oral, rectal, nasal, topical (including buccal and sub-lingual), vaginal or parenteral (including intramuscular, sub-cutaneous and intravenous) administration or in a form suitable for administration by inhalation or insufflation.

The compounds of the invention, together with a conventional adjuvant, carrier, or diluent, may thus be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, in the form of suppositories for rectal administration; or in the form of sterile injectable solutions for parenteral (including subcutaneous) use.

Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

For preparing pharmaceutical compositions from the compounds of the present invention, pharmaceutically acceptable carriers can be either solid or liquid. Solid form preparations include powders, tablets, pills, capsules, cachets, suppositories, and dispensable granules. A solid carrier can be one or more substances which may also act as diluents, flavouring agents, solubilizers, lubricants, suspending agents, binders, preservatives, tablet disintegrating agents, or an encapsulating material.

Suitable vehicles, carriers or excipients include magnesium carbonate, magnesium stearate, talc, sugar, lactose, pectin, dextrin, starch, gelatin, tragacanth, methylcellulose, sodium carboxymethylcellulose, a low melting wax, cocoa butter and the like. The term “preparation” is intended to include the formulation of the active compound with an encapsulating material as the carrier by providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

Liquid form preparations include solutions, suspensions, and emulsions, for example, water or water-propylene glycol solutions. For example, parenteral injection liquid preparations can be formulated as solutions in aqueous polyethylene glycol solution.

Sterile liquid form compositions include sterile solutions, suspensions, emulsions, syrups and elixirs. The active ingredient can be dissolved or suspended in a pharmaceutically acceptable carrier, such as sterile water, sterile organic solvent or a mixture of both.

The compositions according to the present invention may thus be formulated for parenteral administration (for example, by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilisation from solution, for constitution with a suitable vehicle, for example, sterile, pyrogen-free water, before use.

Pharmaceutical forms suitable for injectable use include sterile injectable solutions or dispersions, and sterile powders for the extemporaneous preparation of sterile injectable solutions. They should be stable under the conditions of manufacture and storage and may be preserved against oxidation and the contaminating action of microorganisms such as bacteria or fungi.

The solvent or dispersion medium for the injectable solution or dispersion may contain any of the conventional solvent or carrier systems for the compounds, and may contain, for example, water, ethanol, polyol (for example, glycerol, propylene glycol and liquid polyethylene glycol and the like), suitable mixtures thereof, and vegetable oils.

Pharmaceutical forms suitable for injectable use may be delivered by any appropriate route including intravenous, intramuscular, intracerebral, intrathecal, epidural injection or infusion.

Sterile injectable solutions are prepared by incorporating the active compounds in the required amount in the appropriate solvent with various other ingredients such as these enumerated above, as required, followed by filtered sterilization. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, preferred methods of preparation are vacuum drying or freeze-drying of a previously sterile-filtered solution of the active ingredient plus any additional desired ingredients.

When the active ingredients are suitably protected they may be orally administered, for example, with an inert diluent or with an assimilable edible carrier, or they may be enclosed in hard or soft shell gelatin capsule, or they may be compressed into tablets, or they may be incorporated directly with the food of the diet. For oral therapeutic administration, the active compound may be incorporated with excipients and used in the form of ingestible tablets, buccal tablets, troches, capsules, elixirs, suspensions, syrups, wafers and the like.

The amount of active compound in therapeutically useful compositions should be sufficient that a suitable dosage will be obtained.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin, or a flavouring agent such as peppermint, oil of wintergreen, or cherry flavouring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations, including those that allow specific delivery of the active peptide to specific regions of the gut.

Aqueous solutions suitable for oral use can be prepared by dissolving the active component in water and adding suitable colorants, flavours, stabilising and thickening agents, as desired. Aqueous suspensions suitable for oral use can be made by dispersing the finely divided active component in water with viscous material, such as natural or synthetic gums, resins, methylcellulose, sodium carboxymethylcellulose, or other well-known suspending agents.

Pharmaceutically acceptable carriers and/or diluents include any and all solvents, dispersion media, coatings, antibacterial and antifungal agents, isotonic and absorption delaying agents and the like.

Also included are solid form preparations that are intended to be converted, shortly before use, to liquid form preparations for oral administration. Such liquid forms include solutions, suspensions, and emulsions. These preparations may contain, in addition to the active component, colorants, flavours, stabilisers, buffers, artificial and natural sweeteners, dispersants, thickeners, solubilising agents and the like.

For topical administration to the epidermis the compounds according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Formulations suitable for topical administration in the mouth include lozenges comprising active agent in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension.

In the case of a spray, this may be achieved for example by means of a metering atomising spray pump. To improve nasal delivery and retention the compounds according to the invention may be encapsulated with cyclodextrins, or formulated with other agents expected to enhance delivery and retention in the nasal mucosa.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, a hydrofluorocarbon (HFC) for example hydrofluoroalkanes (HFA), carbon dioxide, or other suitable gas.

The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, for example gelatin, or blister packs from which the powder may be administered by means of an inhaler.

In formulations intended for administration to the respiratory tract, including intranasal formulations, the compound will generally have a small particle size for example of the order of 5 to 10 microns or less. Such a particle size may be obtained by means known in the art, for example by micronization.

When desired, formulations adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of viral infection in living subjects having a diseased condition in which bodily health is impaired as herein disclosed in detail. The invention also includes the compounds in the absence of carrier where the compounds are in unit dosage form.

Liquids or powders for intranasal administration, tablets or capsules for oral administration and liquids for intravenous administration are the preferred compositions.

The compounds as set forth above can be useful in a method of inhibiting RSV or in treating or preventing an RSV infection of other infections caused by related members of the paramyxovirus family such as mumps virus, human parainfluenzaviruses, and Nipah and hendra virus. The reference to RSV as used hereinbelow also include these related members or the paramyxovirus family compounds can also be used to treat an RSV disease or reduce exacerbation of an underlying or pre-existing respiratory disease wherein RSV infection is a cause of said exacerbation. The RSV disease may include bronchiolitis or pneumonia. The underlying or pre-existing respiratory diseases or conditions may include asthma, chronic obstructive pulmonary disease (COPD) and immunosuppression such as immunosuppression experienced by bone marrow transplant recipients.

Treatment may be therapeutic treatment or prophylactic treatment or prevention. Generally, the term “treating” means affecting a subject, tissue or cell to obtain a desired pharmacological and/or physiological effect and includes: (a) inhibiting the viral infection or RSV disease, such as by arresting its development or further development; (b) relieving or ameliorating the effects of the viral infection or RSV disease, such as by causing regression of the effects of the viral infection or RSV disease; (c) reducing the incidence of the viral infection or RSV disease or (d) preventing the viral infection or RSV disease from occurring in a subject, tissue or cell predisposed to the viral infection or RSV disease or at risk thereof, but has not yet been diagnosed with a protective pharmacological and/or physiological effect so that the viral infection or RSV disease does not develop or occur in the subject, tissue or cell.

The term “subject” refers to any animal, in particular mammals such as humans, having a disease which requires treatment with the compound of formula (I). Particularly preferred treatment groups include at risk populations such as hospitalised subjects, the elderly, high-risk adults and infants. In one embodiment of the invention, an effective amount of the above compounds, or pharmaceutical compositions thereof, is administered to a patient or subject in need thereof.

The term “administering” or “administered” should be understood to mean providing a compound or pharmaceutical composition of the invention to a subject suffering from or at risk of the disease or condition to be treated or prevented.

As indicated above, although the invention has been described with particular reference to treating RSV infections and diseases, more particularly human and animal RSV infections or diseases, it will be appreciated that the invention may also be useful in the treatment of other viruses of the sub-family Pneumovirinae, more particularly, the genera Pneumovirus and Metapneumovirus.

### Dosages

The term “therapeutically effective amount” refers to the amount of the compound of formula (I) that will elicit the biological or medical response of a subject, tissue or cell that is being sought by the researcher, veterinarian, medical doctor or other clinician.

By “effective amount” is generally considered that amount that will be effective to treat the condition sought to be treated, or to inhibit RSV, and this effective amount is variable based on a variety of factors including age, size and condition of the patient being treated. Accordingly, one skilled in the art would be readily able to determine the specific effective amount for each patient being treated for RSV, an RSV-related condition, or to inhibit RSV in a given case.

In the treatment of RSV infections or diseases, an appropriate dosage level will generally be about 0.01 to about 500 mg per kg subject body weight per day which can be administered in single or multiple doses. The dosage may be selected, for example, to any dose within any of these ranges, for therapeutic efficacy and/or symptomatic adjustment of the dosage to the subject to be treated.

As indicated above, it will be understood that the specific dose level and frequency of dosage for any particular subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the subject undergoing therapy.

### Methods of Preparation

The compounds of the invention may generally be prepared by one skilled in the art using at least the following methods.

With regard to the compounds as identified above, there are at least three general methods that may be employed, identified herein as general method “A1”, “A2” and “A3”, respectively.

General information regarding method of preparation: In the syntheses of the present invention, all evaporations were carried out in vacuo with a rotary evaporator. Analytical samples were dried in vacuo (1-5 mmHg) at rt. Thin layer chromatography (TLC) was performed on silica gel plates, spots were visualized by UV light (214 and 254 nm). Purification by column and flash chromatography was carried out using silica gel (200-300 mesh). Solvent systems are reported as mixtures by volume. All NMR spectra were recorded on a Bruker 400 (400 MHz) spectrometer. 1H chemical shifts are reported in 5 values in ppm with the deuterated solvent as the internal standard. Data are reported as follows: chemical shift, multiplicity (s=singlet, d=doublet, t=triplet, q=quartet, br=broad, m=multiplet), coupling constant (Hz), integration.

These general methods A1, A2 and A3 are shown schematically as follows:

### General Synthetic Method A1

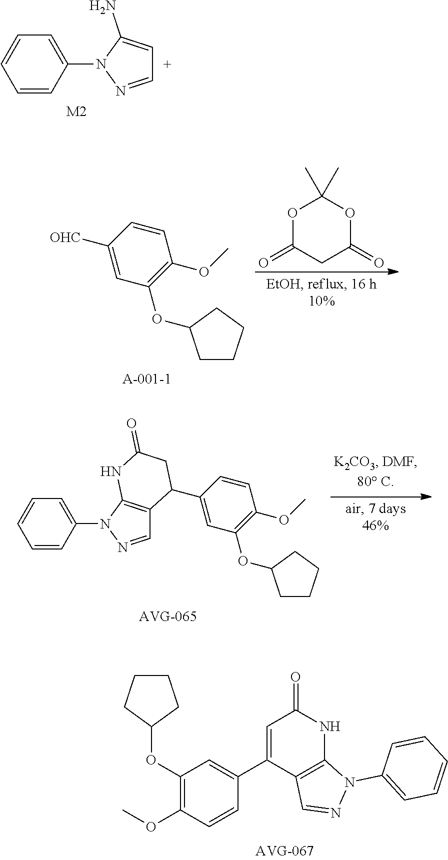

### General Synthetic Method A2

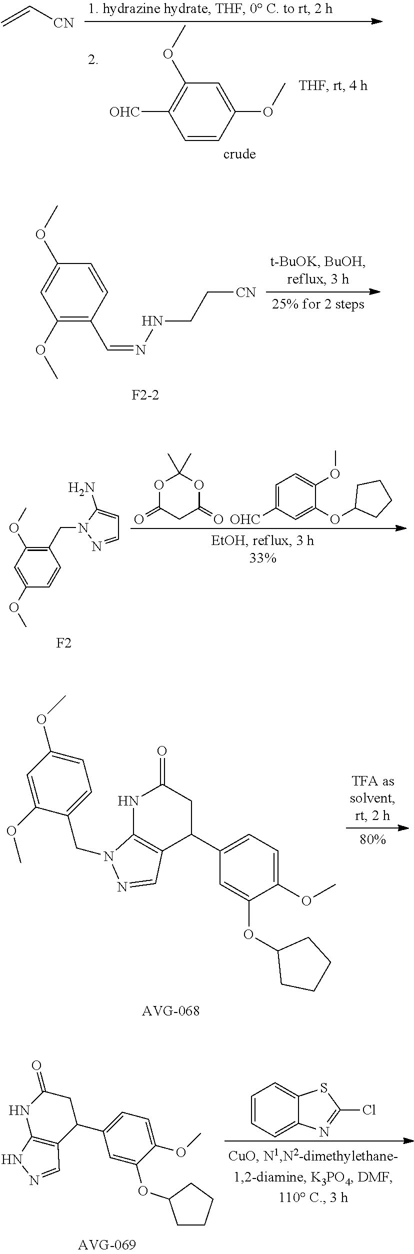

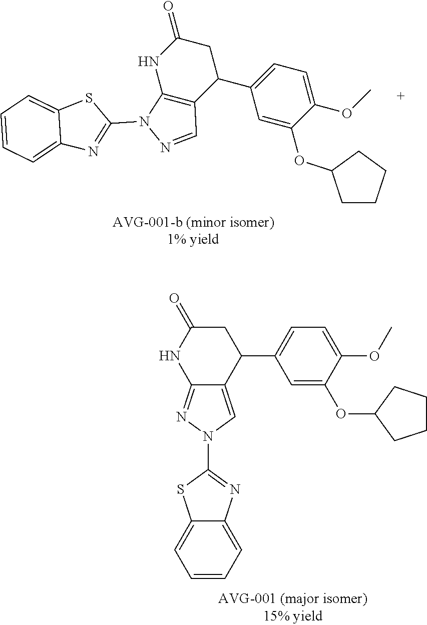 [SMILES: COc1ccc(C2CC(=O)Nc3c2cnn3-c2nc3ccccc3s2)cc1OC1CCCC1 ; COc1ccc(C2CC(=O)Nc3nn(-c4nc5ccccc5s4)cc32)cc1OC1CCCC1]

### General Synthetic Method A3

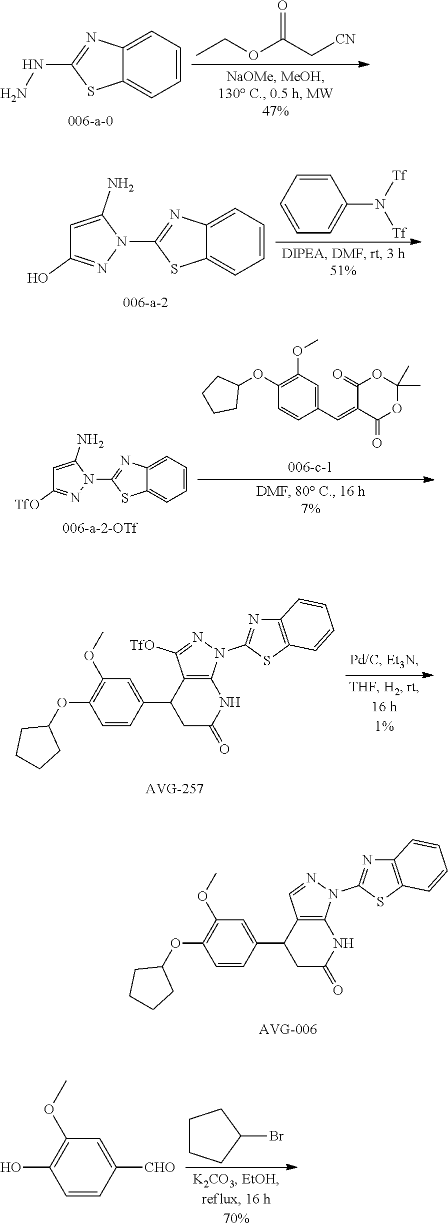

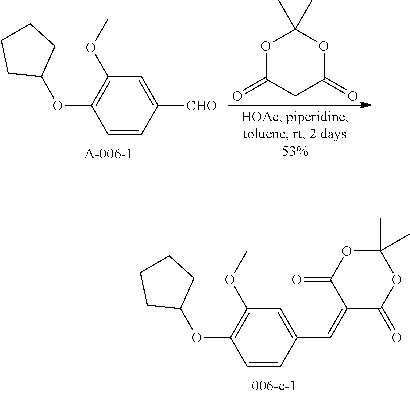

Other compounds within the general formulas of the present disclosure may also be prepared using the following general method “B” for synthesis:

### General Synthetic Method B

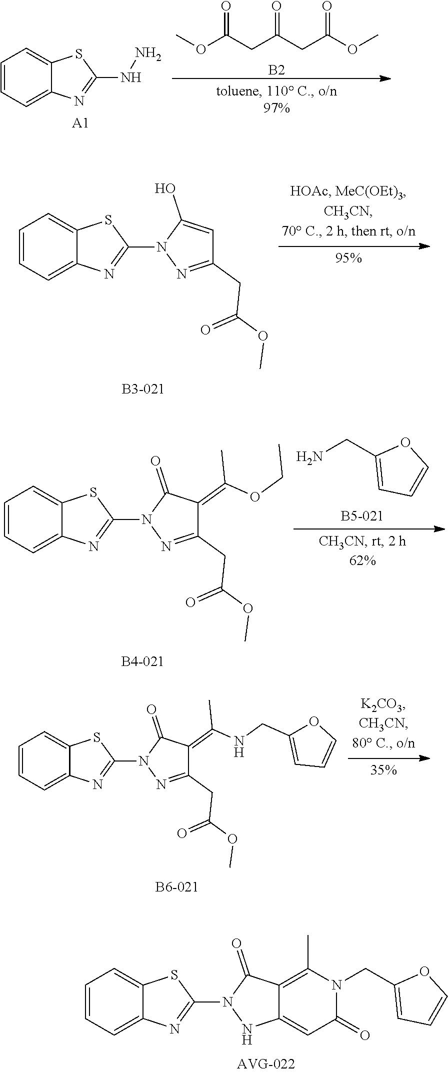

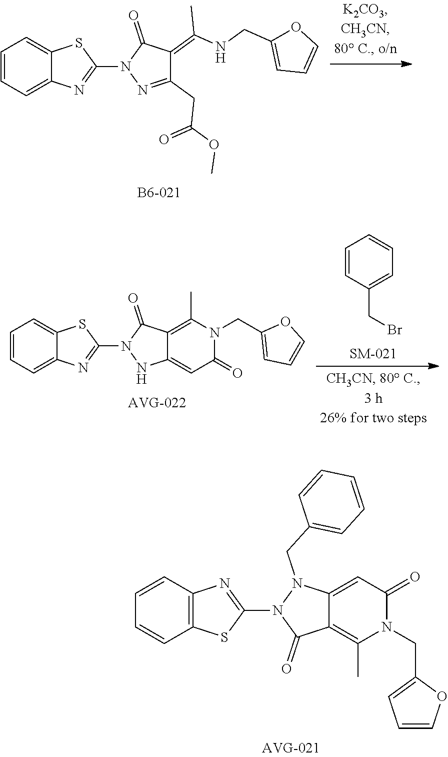 [SMILES: COC(=O)CC1=NN(c2nc3ccccc3s2)C(=O)/C1=C(\C)NCc1ccco1 ; Cc1c2c(=O)n(-c3nc4ccccc4s3)[nH]c2cc(=O)n1Cc1ccco1 ; BrCc1ccccc1 ; Cc1c2c(=O)n(-c3nc4ccccc4s3)n(Cc3ccccc3)c2cc(=O)n1Cc1ccco1]

Still other methods to produce the compounds as set forth above would be well understood by those of ordinary skill in the art.

### Methods of Treatment or Prevention of Viral Respiratory Disease

In accordance with exemplary embodiments of the invention, a method is provided for inhibiting a viral respiratory infection, illness, disease, or other respiratory condition comprising administering to a patient in need thereof, an effective amount of a compound of Formula 1a as defined above, or a pharmaceutical composition comprising an effective amount of the compound of Formula 1a. As indicated above, the respiratory infection, illness, disease, or other respiratory condition may be caused by a virus selected from the group consisting of RSV, coronavirus, SARS-CoV-2, SARS, pneumovirus, paramyxovirus, metapneumovirus, mumps virus, human parainfluenzaviruses, Nipah virus (NIV), and hendra virus.

Still other methods of inhibiting a respiratory infection, illness, disease, or other respiratory condition are provided such as where the compound or composition administered constitutes or includes an effective amount of a compound of formula 1 b or 1 c. In all such cases, as indicated above, the effective amount of the compound or composition would be that generally considered to be the amount that will be effective to treat or prevent the condition sought to be treated, or to cause viral inhibition or impairment, and as recognized by one of ordinary skill in the art, this effective amount will be variable based on a variety of factors including age, size and condition of the patient being treated. Accordingly, one skilled in the art would be readily able to determine the specific effective amount for each patient being treated for a viral respiratory infection, disease, or other respiratory condition caused by a respiratory virus such as RSV or the other respiratory viruses recited above.

Still further, in another exemplary embodiment, a method is provided for treating or preventing a respiratory infection, comprising administering to a patient in need thereof an effective amount of a compound of Formula 1a, Formula 1b, or Formula 1c as shown above, or a pharmaceutical composition that contains said compound. As would be recognized by one of ordinary skill in the art, said compounds can be administered to a patient in need thereof in a number of suitable ways including orally, intravenously, topically, parentally, subcutaneously, intradermally, or by inhalation. Exemplary routes of administration include buccal, oral, intravenous, intramuscular, topical, subcutaneous, rectal, vaginal, parenteral, pulmonary, intranasal, ophthalmic, and the like.

In accordance with the invention, a method is provided for inhibiting or impairing RNA elongation of viral RNA of a virus that causes a respiratory infection, disease, illness, or other respiratory condition, said method comprising administering to a patient in need thereof an effective amount of a compound of Formula 1a, Formula 1 b, or Formula 1 c as reflected above, or pharmaceutical compositions containing said compound. This method could be utilized against a variety of respiratory viruses including those selected from the group consisting of RSV, coronavirus, SARS-CoV-2, SARS, pneumovirus, paramyxovirus, metapneumovirus, mumps virus, human parainfluenzaviruses, Nipah virus (NIV), and hendra virus.

Still further, a method is provided for blocking viral RNA-dependent RNA polymerase of a virus that causes a respiratory infection, disease, or other respiratory condition, said method comprising administering to a patient in need thereof an effective amount of a compound of Formula 1a, Formula 1 b, or Formula 1c as reflected above, or pharmaceutical compositions containing said compound. This method could be utilized against a variety of respiratory viruses including those selected from the group consisting of RSV, coronavirus, SARS-CoV-2, SARS, pneumovirus, paramyxovirus, metapneumovirus, mumps virus, human parainfluenzaviruses, Nipah virus (NIV), and hendra virus. This blocking may be a competitive or non-competitive blocking achieved by administration of the compounds of the present invention to a patient in need thereof.

### EXAMPLES

The following examples are set forth below to illustrate the methods and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1: Assays of Compounds of the Disclosure

Compounds in accordance with the invention were tested for activity with regard to viral loads and titers in mice. This testing involved the following procedure:

### Cells:

HEp-2 cells (ATCC CCL-23) were grown at 37° C. and 5% CO₂ in Dulbecco's modified Eagle's medium (DMEM) supplemented with 7.5% heat-inactivated fetal bovine serum (FBS). Recombinant respiratory syncytial virus strain A2 with line19F and either mKate or FireSMASH reporter genes was rescued and amplified as described previously (Hotard et al., 2012; Yan et al., 2015).

### Activity Assays:

For reporter-based dose-response assays, 3-fold serial dilutions of compounds were prepared in triplicate using a Nimbus liquid handler (Hamilton) and transferred to 96-well plates seeded the day before at 50% confluence in 96-well plate format. Immediately after addition of compound, cells were infected with recRSV-A2line19F-[FireSmash]. At 48 hours post-transfection, luciferase activities of reporter-expressing viruses were determined using ONE-Glo luciferase substrate (Promega) and a H1 synergy plate reader (Biotek). Each plate contained 4 wells each of positive and negative control (infected cells with media containing DMSO or 100 μM cycloheximide, respectively). Normalized luciferase activities were analyzed with the formula: % inhibition=(Signal_{Sample}−Signal_{Min})/(Signal_{Max}−Signal_{Min})×100, and dose response curves were further analyzed by normalized non-linear regression with variable slope to determine 50% effective concentration (EC₅₀) and 95% confidence intervals (CI) with Prism 9.0.1 for MacOS (GraphPad).

### Cytotoxicity Assays

To determine the effect of compound on cell metabolism, HEp-2 cells were seeded at 50% confluence in 96-well plates and were incubated with 3-fold serial dilution of compound from 5 or 50 μM as described, including positive and negative controls for normalization. After 48-hour incubation at 37° C., cells were incubated with PrestoBlue (ThermoFisher Scientific) for 1 hour at 37° C. and fluorescence measured with a H1 synergy plate reader (Biotek). 50% cytotoxic concentrations (CC₅₀) and 95% CIs after normalized non-linear regression and variable slope were determined using Prism 9.0.1 for MacOS (GraphPad).

### Experimental Design of Mice Experiments

6-8-week old female Balb/cJ mice (Jackson laboratory, cat #000651) were housed in an ABSL-2 facility and rested for 4-5 days. For efficacy studies, mice were randomly divided into groups (n=5) and infected intranasally with 5×10⁵ TCID₅₀ (25 μl per nare) of recRSV-A2line19F-[mKate] in PBS while under anesthesia with ketamine/xylazine. Treatment (compound or vehicle) was administrated at 10 h post-infection via oral gavage in a 200 μl suspension of 1% methylcellulose and administrated twice daily. Temperature and food consumption were monitored daily, body weight was determined twice daily. All animals were euthanized at 4.5 days after infection and lungs were harvested. To determine lung viral titers, lungs were weighted and homogenized with a bead beater in 300 μl PBS in 3 bursts of 20 seconds by 5-minute rest on ice after each cycle. Samples were clarified for 5 minutes at 4° C. and 20,000×g, supernatant aliquoted and stored at −80° C. before being titrated by median tissue culture infectious dose (TCID₅₀) normalized per gram of lung tissue and per ml of lysate. RSV viral titers were determined using standard 50% tissue infective dose (TCID₅₀) assay in HEp-2 cells and 96 well plates, with a Spearman and Karber based method using fluorescence for detection.

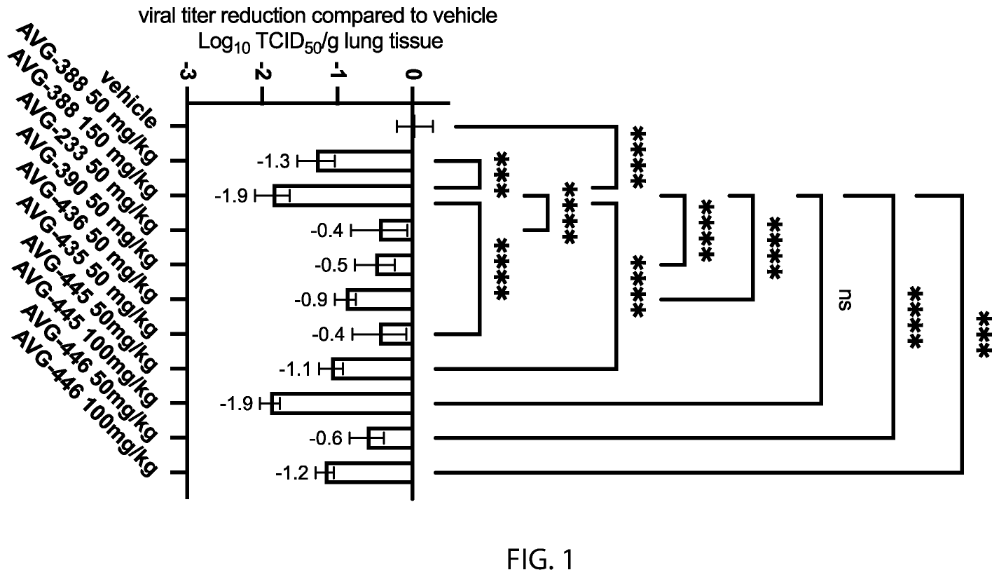

The results of these tests are observed in Table 2 below and in the summary drawing FIG. 1 submitted herewith.

TABLE 2
Formulations of Exemplary Compounds of the Invention and Effect
on In Vivo Lung Titer Reduction in RSV-Infected Mice

| | | | | in vivo lung titer |
|---|---|---|---|---|
| | EC50 95% confidence | | | reduction in RSV- |
| | EC50 | interval | | infected mice |
| compound | (μM) | (μM) | CC50 (μM) | (log 10) |
| AVG-233 | 0.238 | 0.21 to | >45 (max | 50 mg/kg: −0.44 |
| | | 0.27 | concentration | (SD: 0.38) |
| | | | tested) | |
| AVG-388 | 0.053 | 0.045 to | >45 (max | 50 mg/kg: −1.3 |
| | | 0.062 | concentration | (SD: 0.25) |
| | | | tested) | 150 mg/kg: −1.9 |
| | | | | (SD: 0.23) |
| AVG-390 | 0.115 | 0.10 to | >45 (max | 50 mg/kg: −0.50 |
| | | 0.13 | concentration | (SD: 0.26) |
| | | | tested) | |
| AVG-436 | 0.203 | 0.154 to | >5 (max | 50 mg/kg: −0.89 |
| | | 0.266 | concentration | (SD: 0.14) |
| | | | tested) | |
| AVG-435 | 0.085 | 0.067 to | >5 (max | 50 mg/kg: −0.44 |
| | | 0.106 | concentration | (SD: 0.36) |
| | | | tested) | |
| AVG-445 | 0.198 | 0.1483 to | >5 (max | 50 mg/kg: −1.1 |
| | | 0.2592 | concentration | (SD: 0.16) |
| | | | tested) | 100 mg/kg: −1.9 |
| | | | | (SD: 0.13) |
| AVG-446 | 0.236 | 0.1844 to | >5 (max | 50 mg/kg: −0.61 |
| | | 0.2997 | concentration | (SD: 0.23) |
| | | | tested) | 100 mg/kg: −1.2 |
| | | | | (SD: 0.12) |

The above results evidenced that the present compounds as described above could be used to achieve in vivo titer reduction in the lungs of infected mammals, such as RSV-infected mice.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims.

Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

### Example 2: Experiments of the Compounds of the Disclosure Showing the Orally Efficacious Lead Compound of the Claimed Subject Matter Targeting a Dynamic Interface in the RSV Viral Polymerase

### Overview

Respiratory syncytial virus (RSV) is a leading cause of lower respiratory infections in infants and the immunocompromised, yet no efficient therapeutic currently exists. The present inventors have now identified the claimed AVG class of allosteric inhibitors of RSV RNA synthesis. The present experiments were conducted so as to demonstrate through biolayer interferometry and in vitro RNA-dependent RNA-polymerase (RdRP) assays that Applicant's claims AVG compounds bind to the viral polymerase, stalling the polymerase in initiation conformation. Resistance profiling revealed a unique escape pattern, suggesting a discrete docking pose. Affinity mapping using photoreactive AVG analogs identified the interface of polymerase core, capping, and connector domains as molecular target site. A first-generation lead showed nanomolar potency against RSV in human airway epithelium organoids but lacked in vivo efficacy. Docking pose-informed synthetic optimization generated orally efficacious AVG-388, which showed potent efficacy in the RSV mouse model when administered therapeutically. Because of its oral efficacy, AVG-388 shows that the present compounds comprise a significant advance in treatment of RSV and related diseases in a manner not possible using prior compounds. This study maps a druggable target in the RSV RdRP and establishes clinical potential of Applicant's AVG chemotype in accordance with the claimed invention to be used against RSV disease.

### Introduction

As indicated in the above specification, RSV is a viral condition that has caused an estimated 33.1 million cases worldwide in 2015 that required 3.2 million hospitalizations and resulted in 59,800 deaths (1). To address this health threat, a number of vaccine and drug candidates were tested clinically. However, inducing lasting vaccine protection turned out to be challenging (2) and the entry inhibitor presatovir that has completed several phase 2b trials has disappointed in subsequent trials (3, 4). Rapid RSV escape from all advanced entry inhibitor candidate classes through mutations mediating pan-resistance (5) further questions clinical potential of RSV entry inhibition. Biopharmaceuticals for immunoprophylaxis have shown promise (6) and the monoclonal anti-RSV antibody palivizumab is approved for human use, but the high cost of antibody therapy has prohibited broad application. Accordingly, palivizumab is predominantly reserved for high-risk patients such as the immunocompromised and infants born prematurely or with congenital heart or lung disease (7).

To overcome the limitations of RSV entry inhibitors (8), drug developments efforts targeting the viral RNA-dependent RNA polymerase (RdRP) complex have intensified, motivated by the prospect of a broader window of opportunity through interference with both viral genome replication and transcription. Composed of the viral large (L) and phospho- (P) proteins, all enzymatic centers of the RdRP complex that are necessary for RNA synthesis, polyadenylation, capping and cap-methylation of viral transcripts are located in the L protein (9). Template for replicase and transcriptase is a non-segmented, single stranded 15 kilobase RNA of negative polarity that is encapsidated by the viral nucleoprotein (N) (10). Accordingly, RdRP bioactivity depends on multiple intra- and intermolecular protein-protein interfaces to enable interactions between P and L (11), the P-L complex and the N encapsidating genomic viral RNA (12), P-L and the viral anti-termination cofactor M2-1 (13), and P-L and host cell co-factors (14-16).

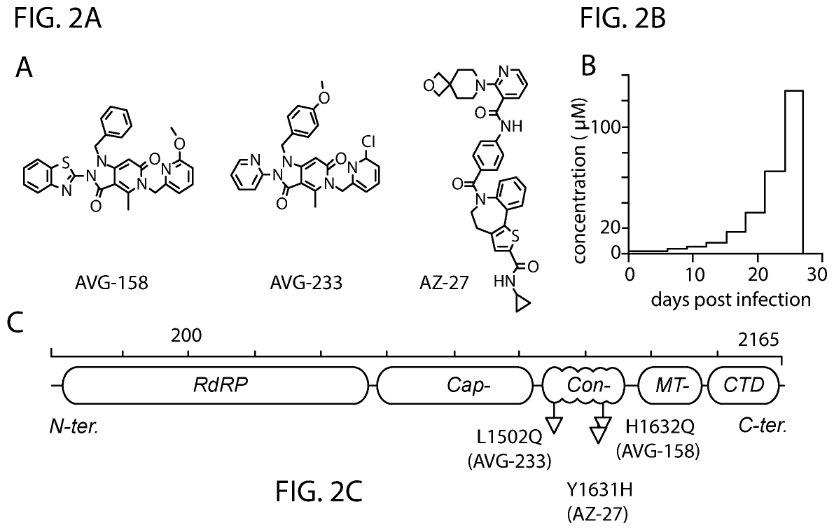

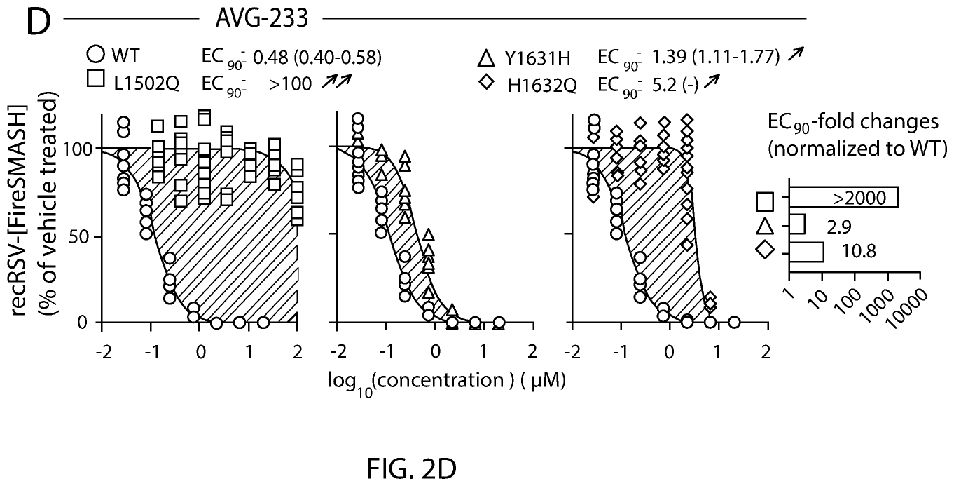

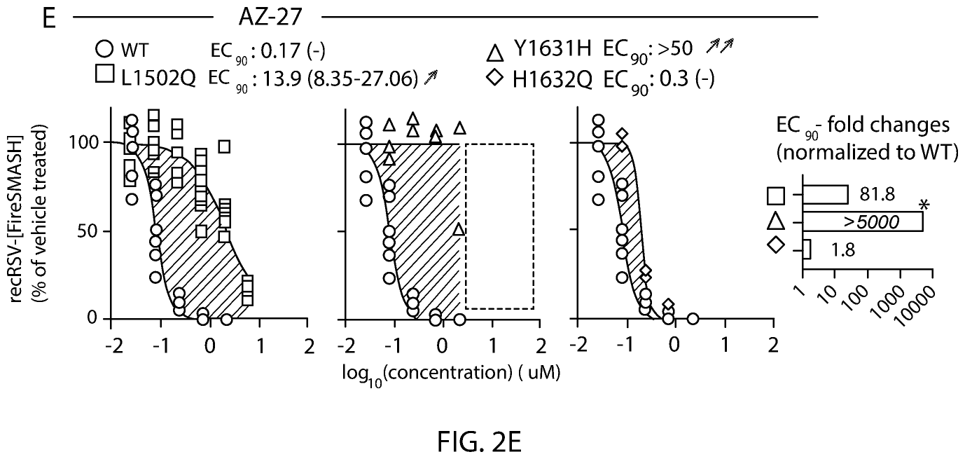

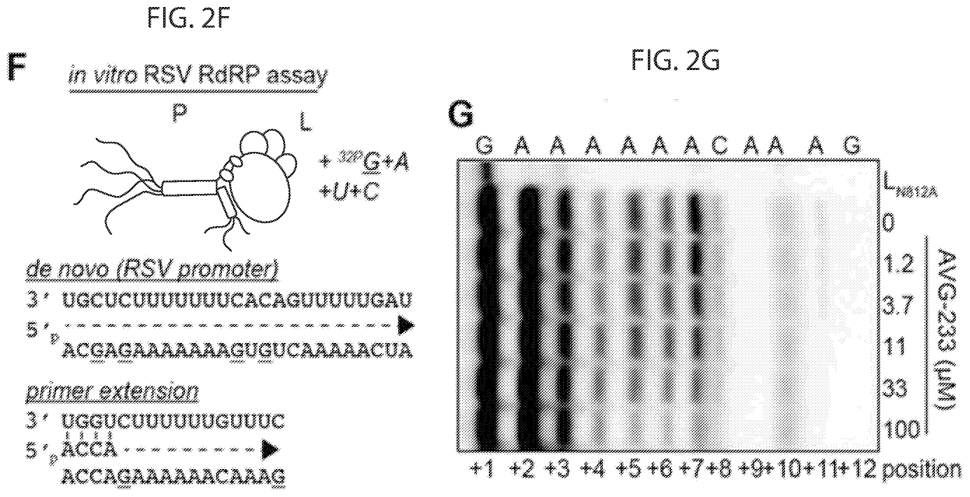

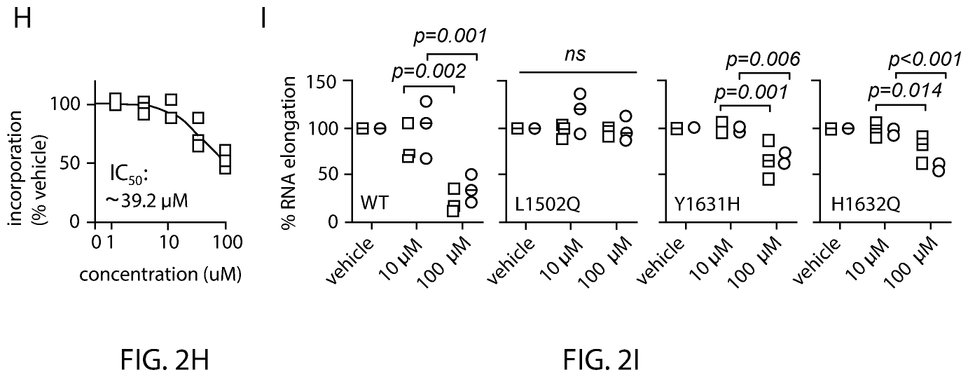

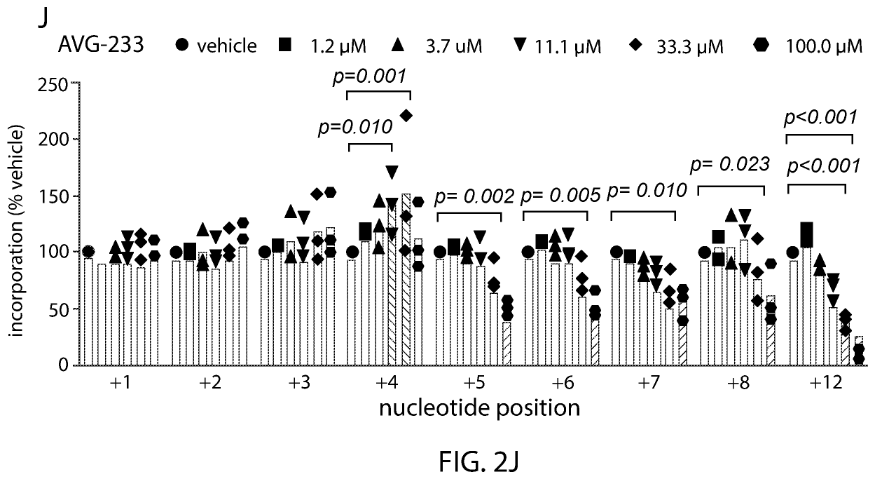

In the last decade, structural insight was gained in the spatial organization of the RSV L protein (17, 18), which was in all cases complexed with a fragment of P. Although some experimental RSV polymerase inhibitors are considered to interact with the N (19) or M2-1 proteins (20), most developmental candidates are thought to target the L protein directly (21, 22). The two RSV L structural models offer a clear view of the core RdRP and capping domains. However, these L structures are thought to represent the conformational state of active transcription, based on the position of a “priming” loop away from the polymerase catalytic center. Conformational insight into L in initiation configuration has remained elusive, since flexibility of connector and methyltransferase domains located downstream of the capping domain prevented structural characterization. These limitations have impaired the mechanistic understanding of allosteric polymerase inhibitors that are associated with resistance mutations located in the C-terminal regions of L (FIG. 2A), such as AstraZeneca's inhibitor AZ-27 (23).

We have recently identified a unique chemotype that potently blocks RSV RdRP bioactivity (24). A first generation lead compound of this class, AVG-233, possesses nanomolar activity against a panel of clinical RSV isolates and has a promising selectivity index (SI=CC₅₀/EC₅₀)>1660. In preparation of formal development, this study has subjected the AVG chemotype to target site identification, characterization of the molecular mechanism of action, and efficacy profiling in primary human airway epithelium organoids and in vivo, resulting in the identification of an orally efficacious developmental lead.

### Results

We have already demonstrated that compounds of the AVG series potently inhibit RSV RNA synthesis (24), but it remained unknown which compounds within this series were able to work best in order to provide an effective treatment regimen against RSV and other respiratory illnesses. It was also unknown what the molecular target of this class was. To identify the molecular target of this class, we resistance profiled the chemotype through serial passaging of a recombinant RSV expressing a red fluorescent protein (recRSV-mKate) in three independent lines, each in the presence of increasing concentrations of either AVG-233 in accordance with the present claims or an earlier developmental intermediate of the series, AVG-158 (FIG. 2A). Virus populations were considered adapted when inhibitor concentrations exceeding the original EC₅₀ value by 100-fold were tolerated (FIG. 2B).

### Unique Resistance Profile of the AVG Series Chemotype

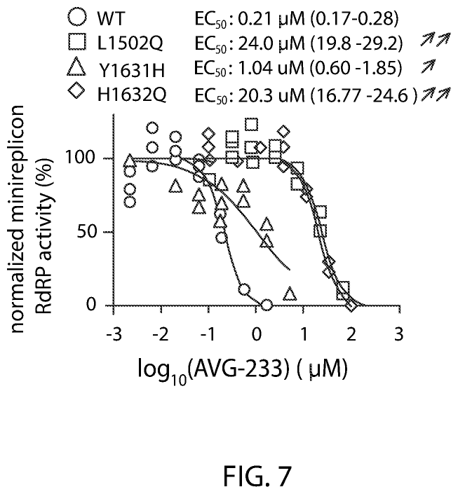

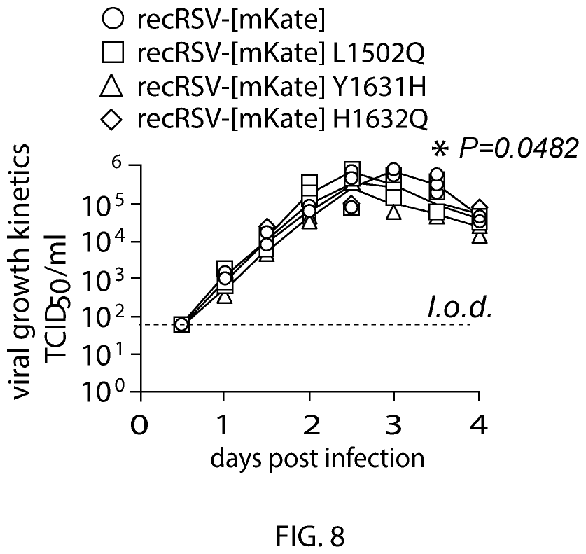

Sanger sequencing revealed a unique substitution in the L ORF for each lineage that affected either residues 1502 (L_{L1502Q}) or 1632 (L_{H1632Q}) (FIG. 2C). Resistance site L_{H1632Q} is located in immediate proximity to a known LY1631H hot-spot that is reportedly involved in escape from several chemically unrelated allosteric RSV polymerase inhibitor classes such as the benzothienoazepines YM-53503 (25), AZ-27 (23), PC786 (26) and the AstraZeneca inhibitor cpd1 (27). We therefore re-engineered L_{L1502Q}, L_{H1632Q}, and L_{Y1631H} independently in RSV L expression plasmids and recRSV, and determined susceptibility to both AVG-233 and AZ-27 in minigenome and/or virus dose-response inhibition assays (FIG. 7). In the minigenome assays, the L_{L1502Q} and L_{H1632Q} mutations caused a ≥100-fold increase in EC₅₀, whereas L_{Y1631H} had only a minor (approx. 5-fold increase) effect (FIG. 7). Rebuilding of the mutations in recRSV did not adversely affect viral growth rates and maximal progeny titers reached in multi-step growth curves (FIG. 8). Consistent with the minireplicon data, the L_{L1502Q} caused a >2000 fold increase in EC₉₀ against AVG-233, whereas L_{Y1631H} barely conferred resistance to the compound (FIG. 2D). However, the L_{H1632Q} mutation had only a moderated effect in the context of recRSV infection, increasing in EC₉₀ values only by approximately 10-fold. Resistance profiles of AZ-27 drew a mirror image of those of AVG-233; the L_{Y1631H} substitution resulted in robust resistance (>2000-fold increase in EC₉₀) consistent with previous reports (23), whereas L_{H1632Q} did not mediate viral escape and L_{L1502Q} had only a moderate effect (FIG. 2E).

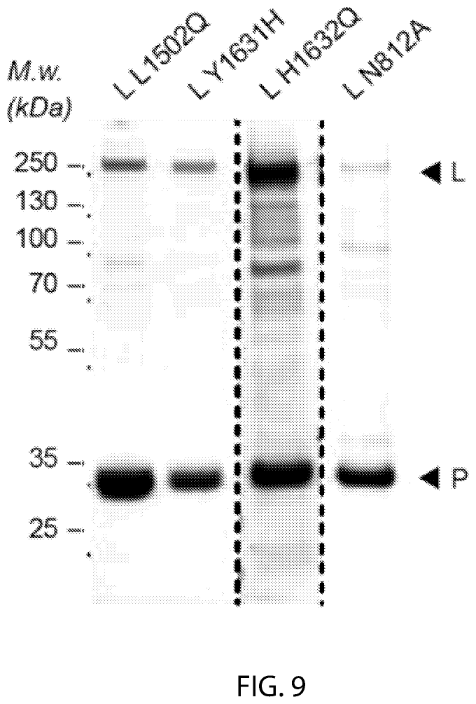

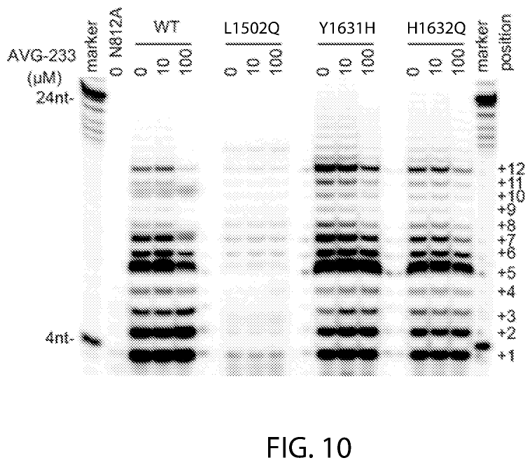

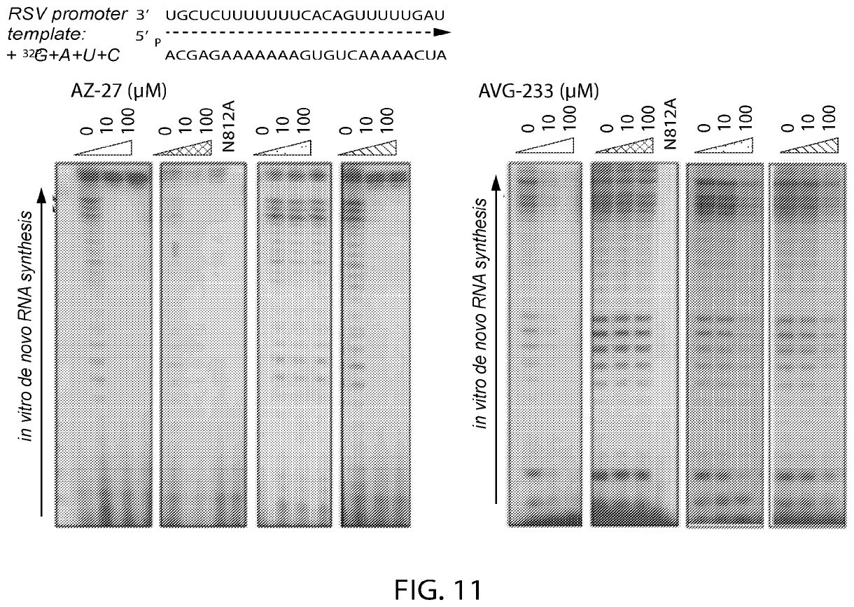

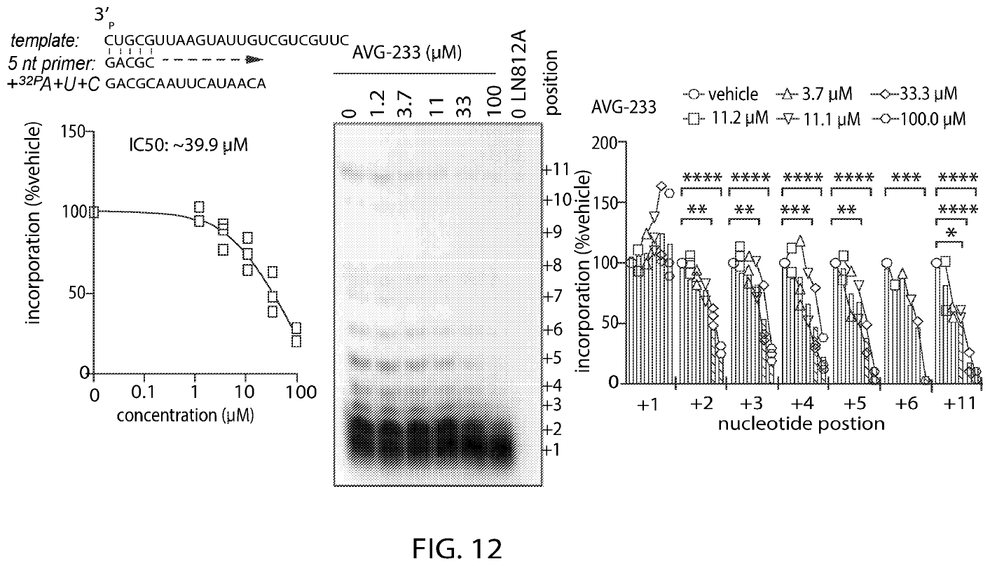

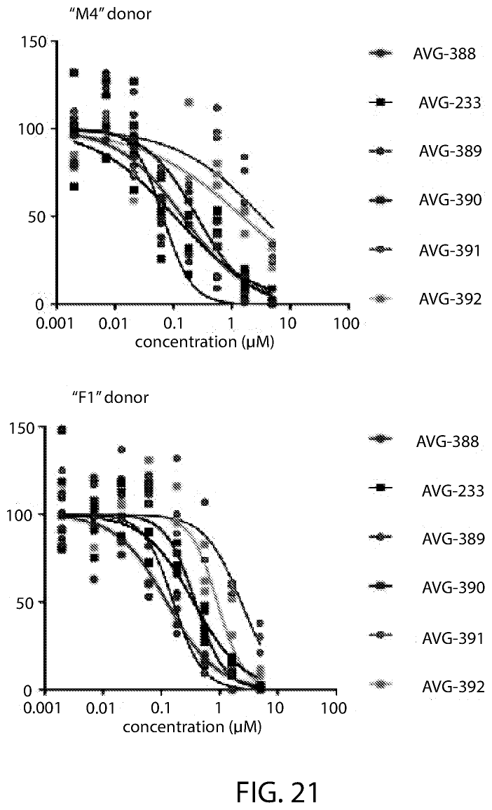

To better understand the molecular basis for L inhibition and resistance, we purified RSV P-L polymerase complexes harboring the different substitutions (FIG. 9) and subjected the preparations to in vitro RdRP assays using synthetic RNA templates and a ³²P-GTP tracer (FIG. 1F). Consistent with previous observations (24), AVG-233 dose-dependently impaired RNA elongation of synthetic primer/template RNA pairs (FIG. 2G), exhibiting a +39 μM IC₅₀ (FIG. 1H). RNA incorporation in vitro was reduced to 36% (±14.6) in the presence of 100 μM AVG-233, but the L1502Q escape mutation restored activity to 98.7% (±12.9), indicating robust resistance (FIG. 21) (FIG. 10). By comparison, Y1631H and H1632Q mediated only moderate escape from AVG-233, represented by restoring polymerase activity to 69% (±5.1) and 60% (±4.0) of untreated, respectively. Similar resistance profiles were observed when using a single stranded RSV promoter as a template to initiate de novo RNA synthesis at the promoter. RNA incorporation after initiation was reduced to 21.7% (±11.8), 96.6% (±4.0), 66.0 (±20.8) and 79.7% (±14.5) when AVG-233 was added to complexes containing L, L_{L1502Q}, L_{Y1631H}, or L_{H1632Q}, respectively (FIG. 2I) (FIG. 11). The arrest of polymerization occurred predominantly after incorporation of four nucleotides and thus after an initial delay (FIG. 2J). However, we noted some variation in the extent of the delay depending on primer/template sequence (FIG. 12).

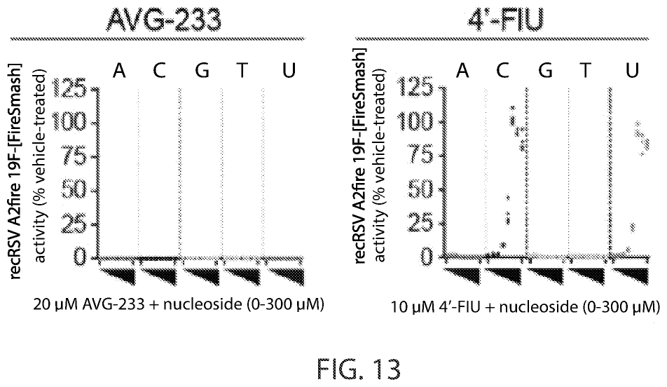

To test for competition of AVG-233 with endogenous RSV polymerase substrates, we added increasing amounts of exogenous nucleosides to the culture media of RSV-infected cells grown in the presence of a sterilizing concentration of AVG-233 (20 μM). In contrast to the known competitive inhibitor 4′-FIU (22), AVG-233 inhibition of RSV replication was unaffected by exogenous nucleosides, indicating that AVG class compounds block the viral RdRP by a non-competitive mechanism (FIG. 13). These results define a non-competitive blockage of RNA synthesis after the initiation step as the primary mechanism of L inhibition by AVG-233. Whereas mechanistically similar to AZ-27, the non-overlapping resistance profiles indicate distinct docking poses.

### Positive AVG-233 Target Identification

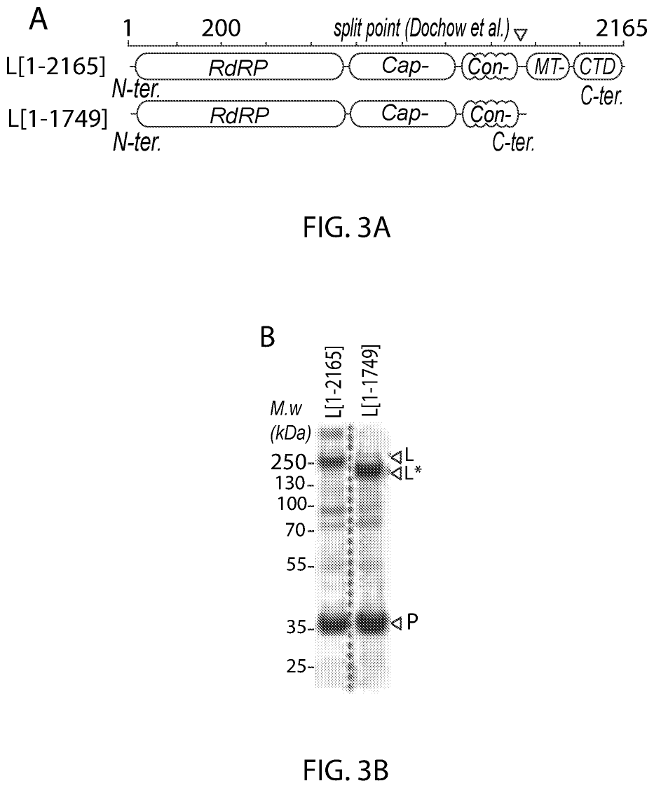

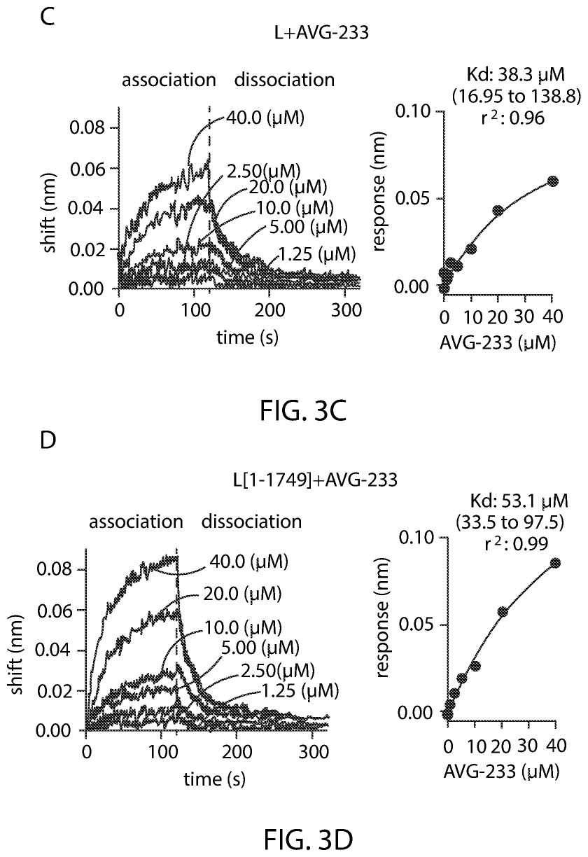

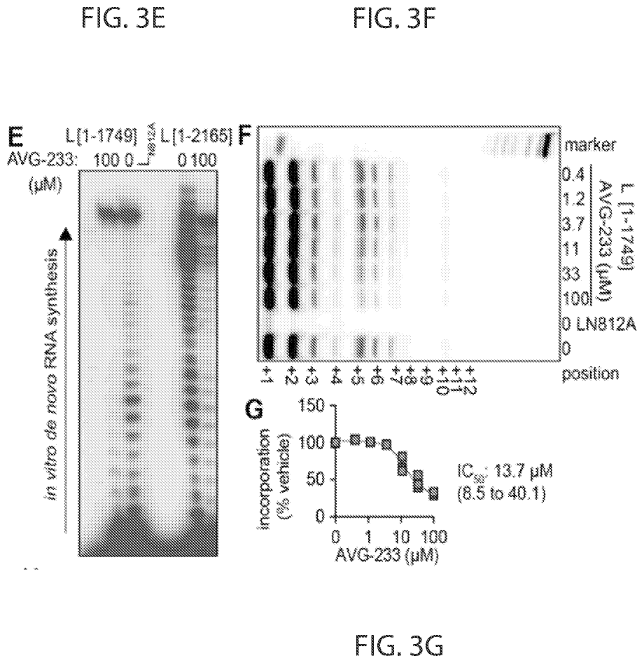

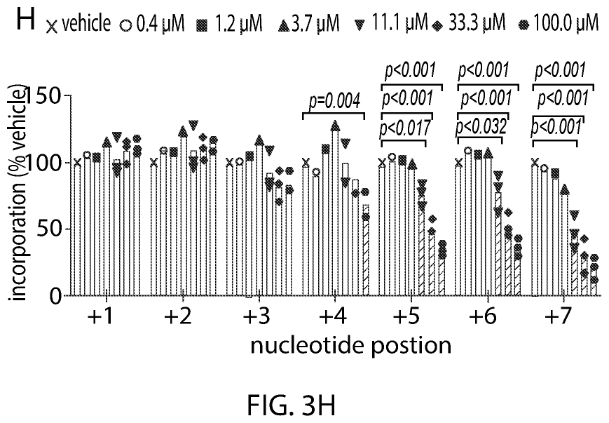

A biologically active RSV polymerase can reconstitute from two independently expressed fragments that are split between the connecting and MTase domains (28) (FIG. 3A), indicating that each of these individual fragments retains folding competence. Using baculovirus/insect cell protein expression, we prepared full-length L and the larger, N-terminal fragment of the split L (residues L₁₋₁₇₄₉) that harbors all of the RdRP domain and all AVG-233 resistance sites (FIG. 3B). Direct binding of AVG-233 to these L preparations was tested through biolayer interferometry (BLI). Both standard L and the L₁₋₁₇₄₉ fragment bound AVG-233 with similar affinities (Kos were 38.3 μM (16.9-138.8) and 53.1 μM (33.5-97.5), respectively) (FIG. 3C,D), providing positive confirmation of L as the molecular target of the compound and demonstrating that the inhibitor binding site is fully present in the L₁₋₁₇₄₉ fragment.

Since this fragment contains the catalytic site for phosphodiester bond formation (17, 18, 29), we explored its ability to synthesize RNA in vitro, using the biochemical RdRP assay set-up. Consistent with proper folding into a native conformation, the L₁₋₁₇₄₉ subunit was RNA synthesis-competent in the biochemical assay (FIG. 3E), although with a noticeable drop in processivity after the ninth incorporated nucleotide compared to full-length L. Importantly, this L fragment was equally susceptible to inhibition by AVG-233 in de novo RNA synthesis (FIG. 3E), confirming productive inhibitor binding.

When testing primer/template based RNA elongation by the L1-1749 fragment, we observed dose-dependent inhibition by AVG-233 with an IC₅₀ of 13.7 μM, (FIG. 3F,G) which did not significantly differ from the value (IC₅₀ 39.2 μM) calculated for full-length L (FIG. 3G,H). AVG-233 suppression of RNA synthesis by the L1-1749 fragment was again delayed, since a significant inhibitory effect was first detectable after incorporation of four nucleotides (FIG. 3H).

### Photoaffinity-Based AVG-233 Target Site Mapping

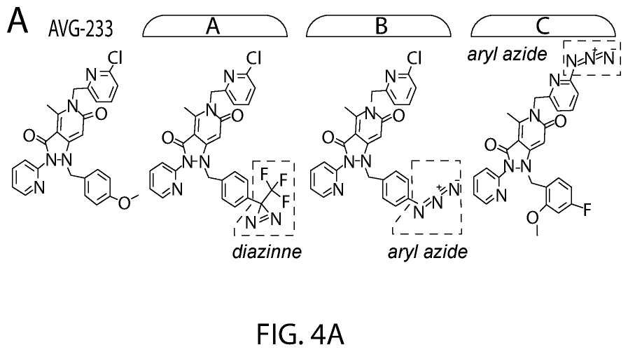

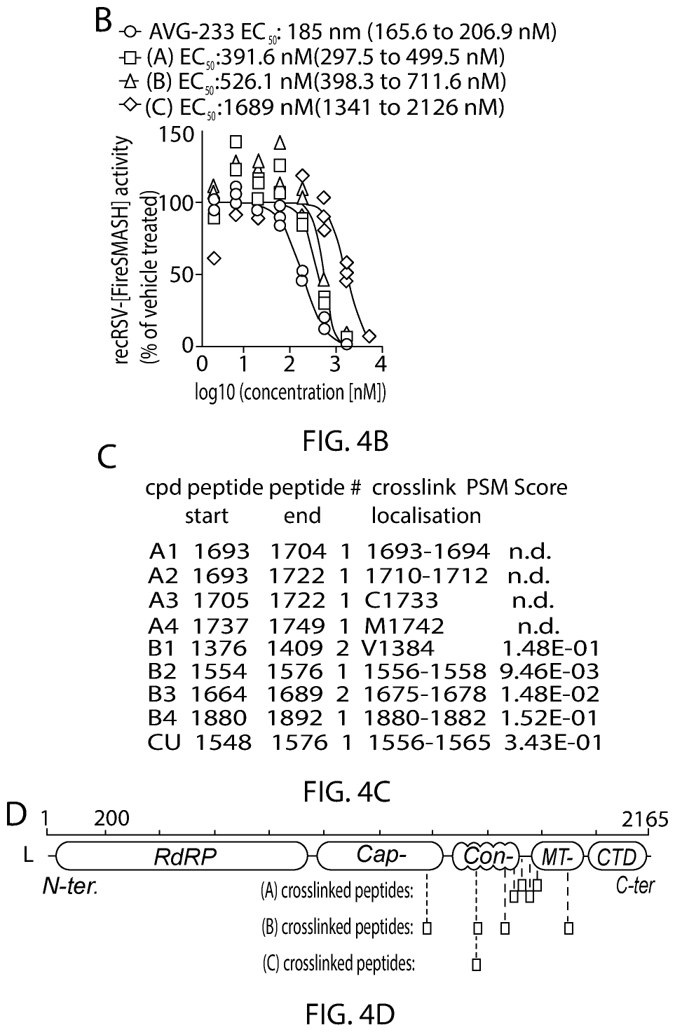

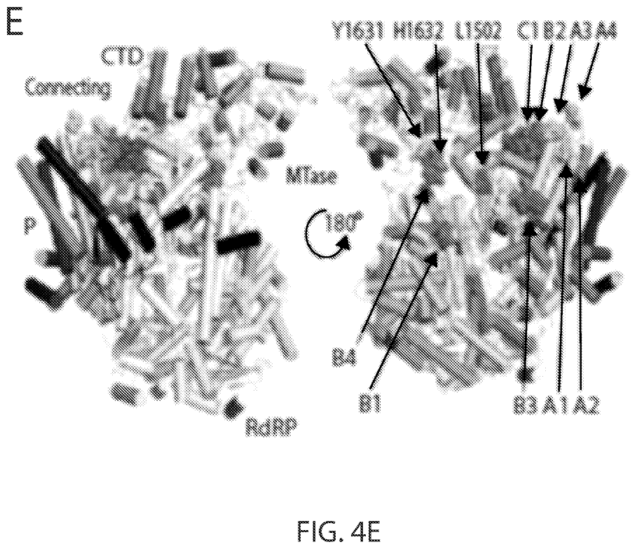

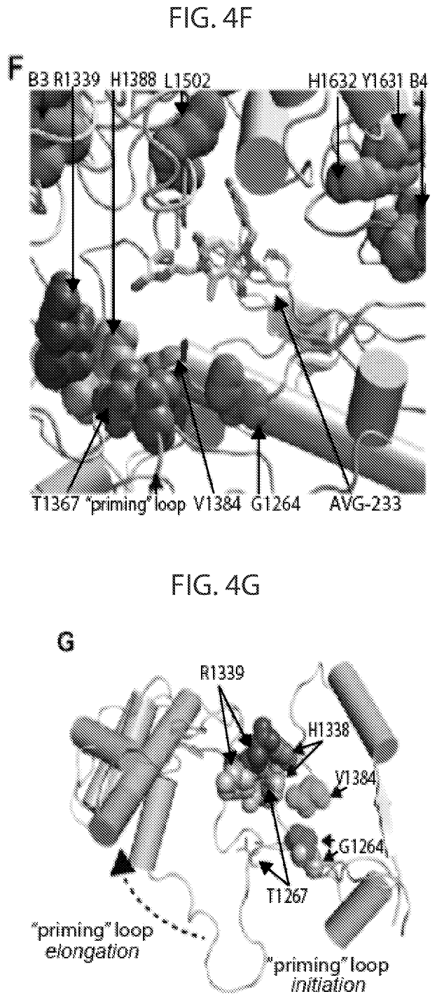

To map the molecular target site of AVG-233, we developed three chemical analogs of the compound capable carrying diazirine or aryl azide moieties that become covalently reactive when photoactivated through exposure to high-energy UV light (FIG. 4A). The design of these analogs was guided by the 3D-quantitative structure-activity relationship (3D-QSAR) model that we have developed for the AVG-233 chemotype (24). Consistent with QSAR predictions, all three analogs retained bioactivity in cell-based assays without photo-activation with only minor (approximately 2- to 10-fold) potency penalties (FIG. 4B). Mass spectrometry analysis after photo-crosslinking of these analogs to purified RSV polymerase and trypsin digestion of the covalent complexes identified for each compound a narrow set of peptides (one to four) with covalently bound ligand, consistent with high target site specificity of the cross-links (FIG. 4C). Specifically, analog A labeled a cluster of four peptides that was located in L region 1693-1749, which is thought to be part of a flexible linker between the connecting and methyltransferase domains (30) (FIG. 4D). Analog B also highlighted 4 peptides, in this case spanning a larger area composed of the L capping (1376-1409), connecting (1554-1576 and 1675-1678) and MTase (1880-1892) domains. Analog C identified a single peptide located in the L connecting domain (1548-1576).

When projected on the structural model of the RSV L core polymerase domain (17, 18), only peptide 1376-1409 could be directly observed due to poor structural resolution of the L C-terminal domain. We therefore generated a homology model of the C-terminal residues of RSV L based on the coordinates reported for the related VSV and Rabies L proteins (31-33). This model posited the AVG-233 resistance mutations and nearly all proximity residues identified through photoaffinity labeling at an interface formed by the L capping, connecting, and MTase domains (FIG. 4E). Data-guided in silico docking of AVG-233 into this site suggested that the compound may broadly restrict structural flexibility of this interface or specifically interfere with conformational rearrangement of the L priming-capping loop (FIG. 4F,G) that facilitates polymerase switch from RNA synthesis initiation to elongation mode (33) through large spatial movement.

### Efficacy of AVG-233 in Air-Liquid Interface Cultures of Primary Epithelium

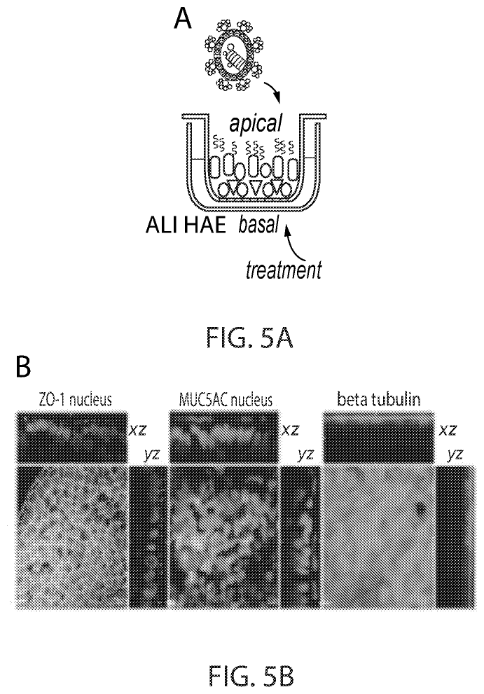

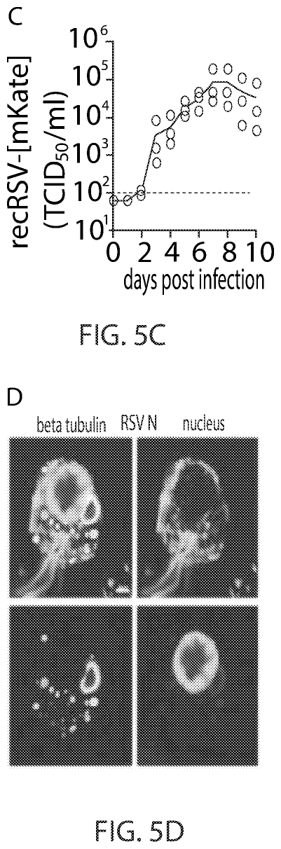

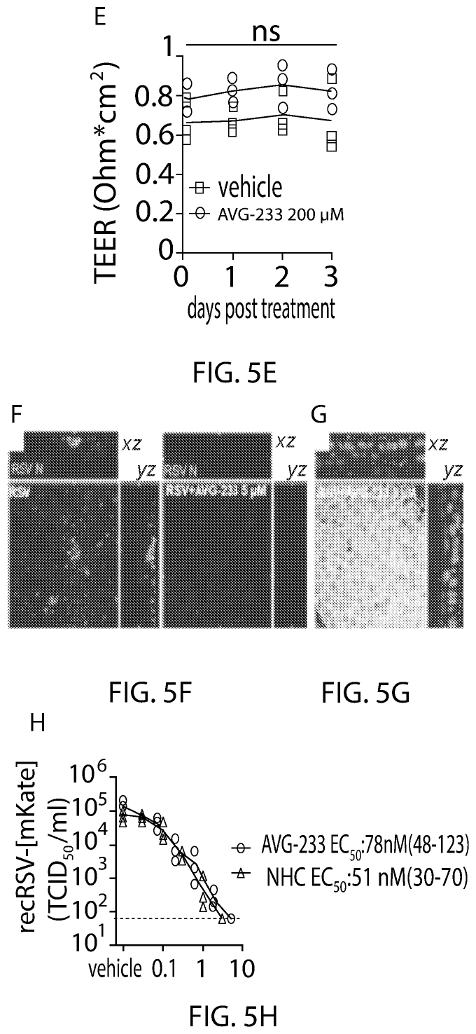

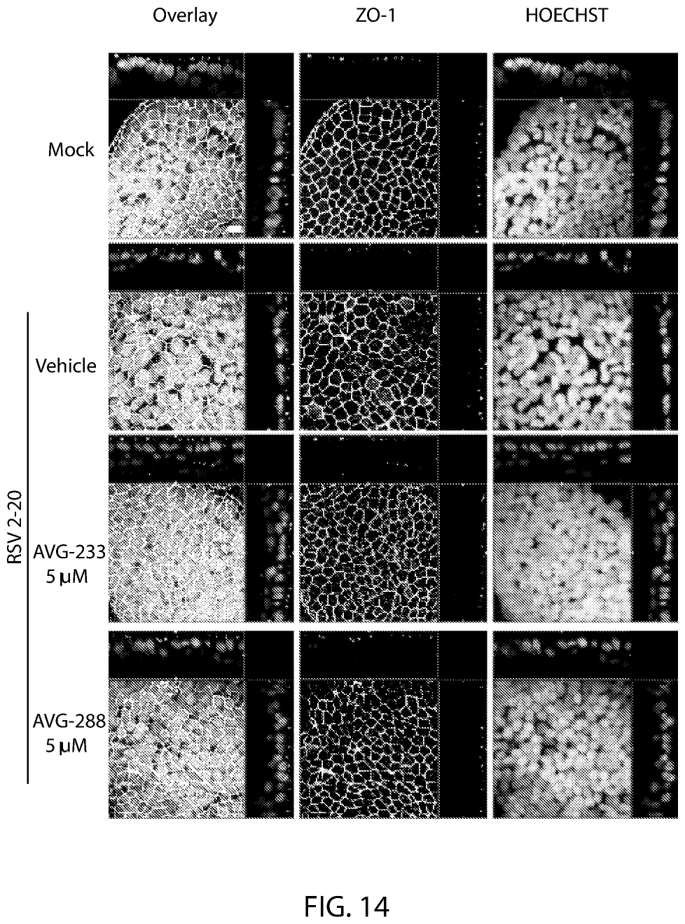

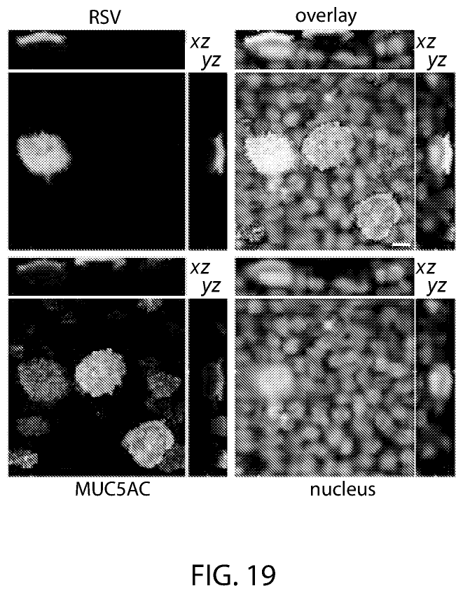

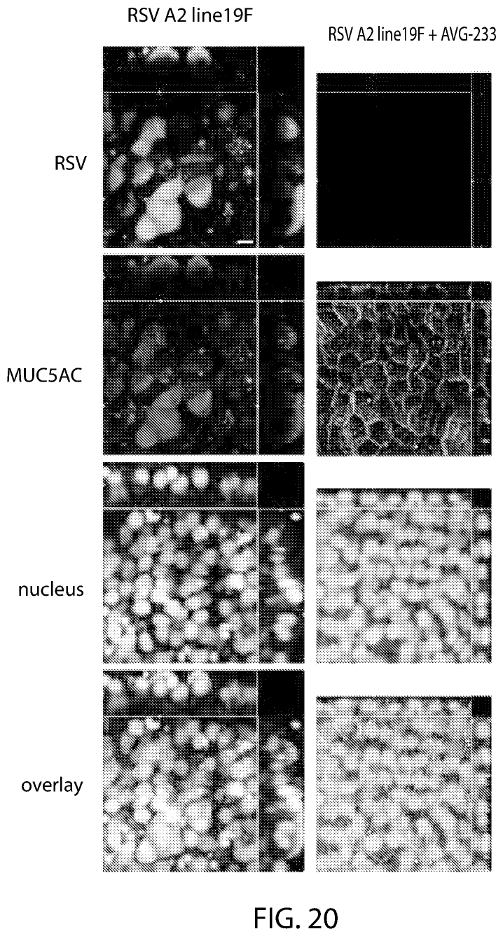

Well-differentiated human airway epithelium organoids grown at air-liquid interface (FIG. 4A) represent a premier model to test potency of antivirals directed, for instance, against RSV (34) or influenza viruses (35) in disease-relevant primary human tissues. These cultures develop tight junctions and feature, amongst others, into mucus producing goblet and ciliated cells (FIG. 4B) (FIG. 14-18). Following apical infection with recRSV-mKate, we noted sustained, robust virus replication resulting in peak shedding of approximately 1×10⁵ infectious particles into the apical space per day over a 10-day experimental window (FIG. 5C). Consistent with previous reports (36), virus replicated predominantly in ciliated cells (FIG. 19), forming characteristic (37) cytoplasmic inclusions (FIG. 5D). Our previous studies have also demonstrated that AVG-233 is well-tolerated without any signs of cytotoxicity at concentrations of 100 μM. Continued multi-day exposure of the airway epithelium cultures to 200 μM compound had no negative effect on tissue organization and tight junction integrity, based on confocal microscopy after ZO—I immunostaining (FIG. 20) and stable trans-epithelial electrical resistance (TEER) between the basolateral and apical chambers (FIG. 5E). Basolateral AVG-233 at 5 μM strongly reduced RSV protein expression (FIG. 5F) without detectable negative effect on epithelium integrity (FIG. 5G). Dose-escalation resulted in gradual inhibition of apical RSV shedding, reaching sterilizing conditions at approximately 3 μM (FIG. 5H). Regression modeling revealed a half-maximal effective concentration (EC₅₀) of 0.07 μM, which closely resembled that of NHC (EC₅₀: 0.05 μM), the parent molecule of the recently authorized broad-spectrum inhibitor molnupiravir (38, 39). These performance parameters correspond to a robust SI >2,850 of AVG-233 in the human airway organoids.

### Development of Orally Efficacious Developmental Candidates of the AVG Series

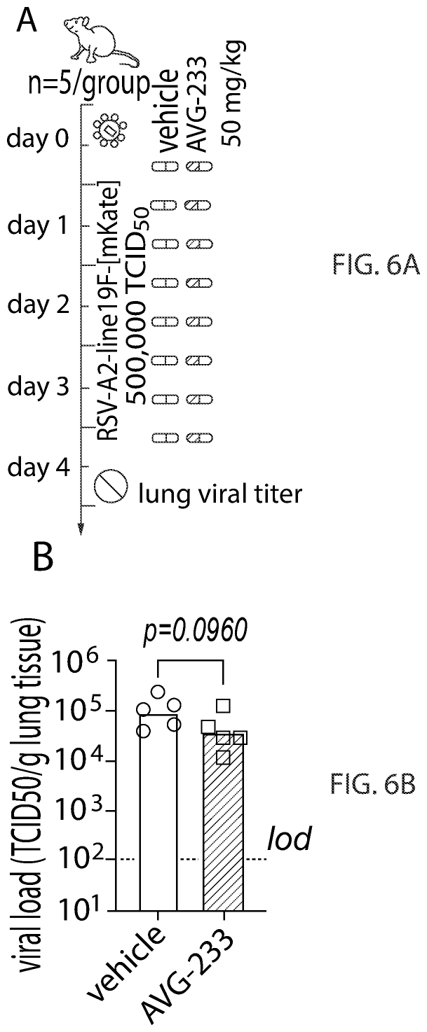

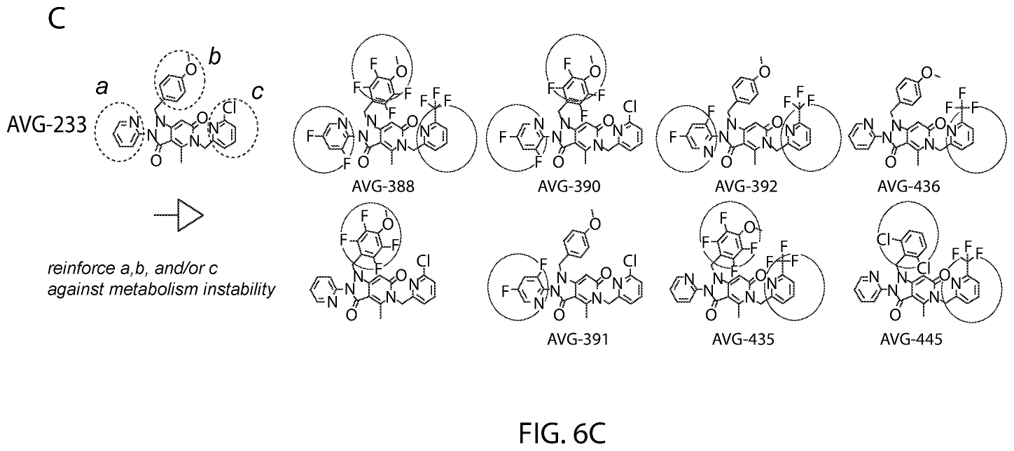

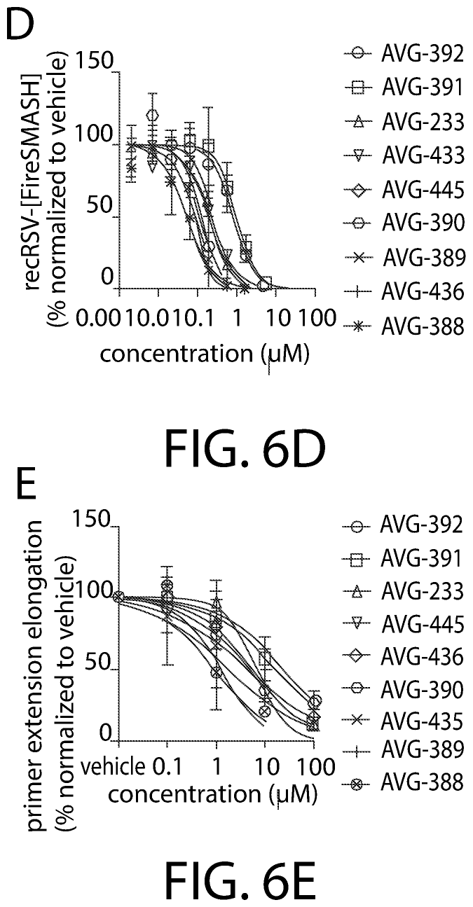

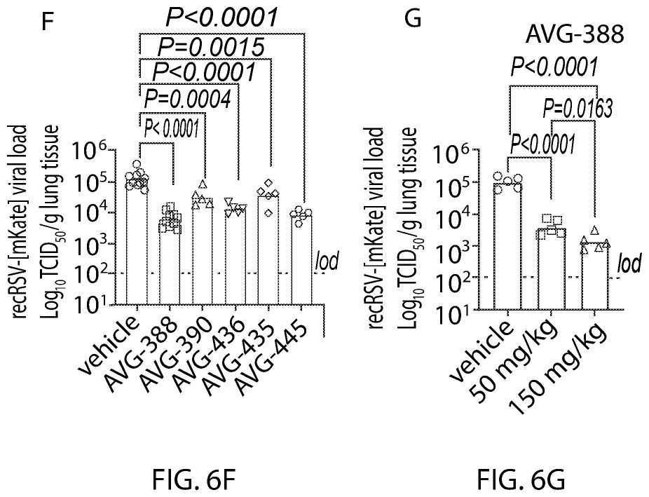

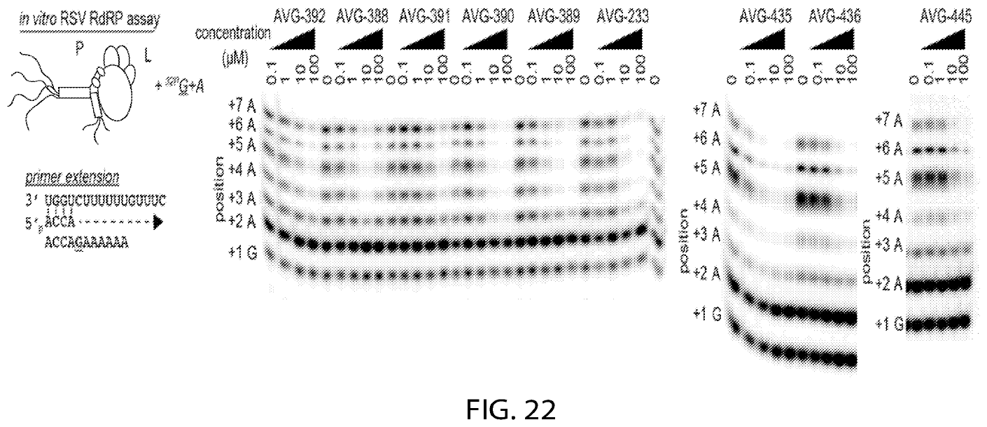

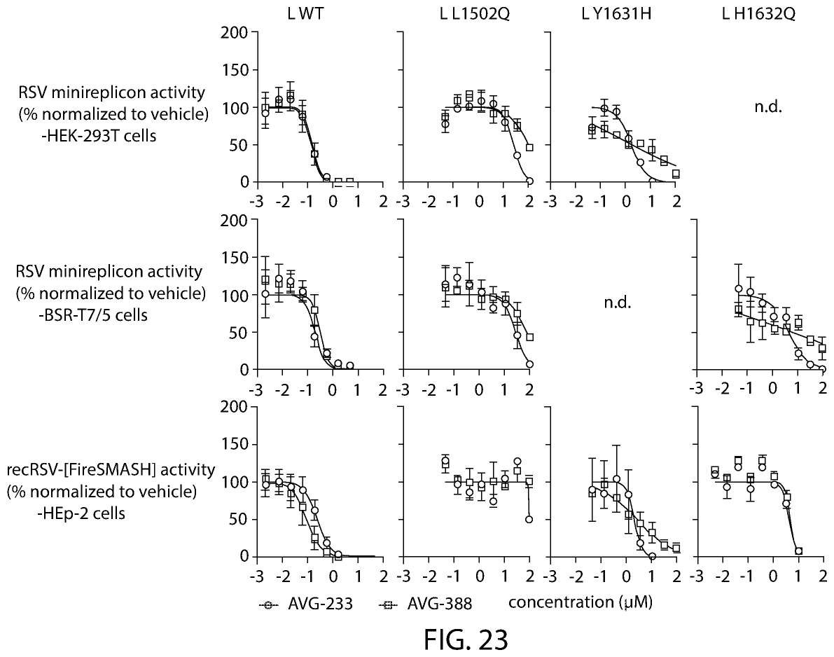

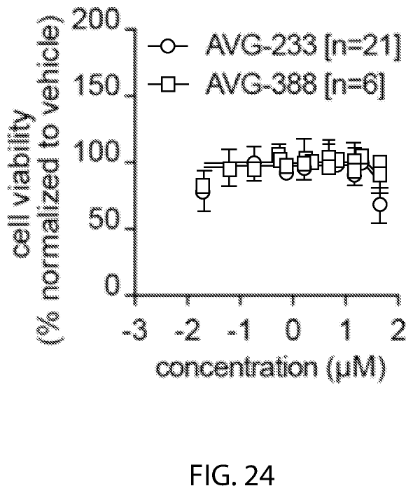

When examining in vivo efficacy of AVG-233 in the mouse model of RSV infection, we noted that neither prophylactic nor therapeutic administration of the compound at a twice-daily dose (b.i.d.) of up to 200 mg/kg significantly reduced lung virus load (FIG. 6A,B). We previously reported that AVG series compounds are rapidly metabolized in mice lung microsomes (24). To minimize potential metabolic liabilities, we designed a series of eight analogs with fluorine atoms introduced to block potential sites of oxidative hydroxylation on the (a) and (b) rings, and/or substitution of the chemically labile ortho-chloropyridine (c) ring (40-42) with a more inert ortho-trifluoromethyl pyridine (FIG. 6C). This series retained potent antiviral activity in cell culture (FIG. 6D) (Table 3) and primary HAE cells (FIG. 21) (Table 3). Mechanism of action was unchanged from that of AVG-233 (FIG. 6E) (FIG. 22) and even the substantially modified analog, AVG-388, shared resistance profile (FIG. 23) (Table 4) and very low cytotoxicity (FIG. 24) with AVG-233, and exhibits superior viral activity.

To assess the effect of these substitutions on in vivo efficacy, we tested the six most potent analogs in the RSV mouse model. Compounds were administered orally at 50 mg/kg body weight in a b.i.d. regimen, starting 12 hours after infection (FIG. 6F). Replacing the ortho-chloropyridine in ring (c) of AVG-233 with a trifluoromethyl group (AVG-436) caused a statistically significant reduction in lung viral load of 0.89 (±0.14) log 10 TCID₅₀/ml (FIG. 6F) (Table 5). A slightly lower antiviral effect resulted from fluorine substitutions on both the a and b moieties (AVG-390). Combining these strategies, fluorines on the (a), (b), and (c) rings (AVG-388) yielded strong antiviral efficacy with a lung virus load reduction of 1.3 (±0.25) log 10 TCID₅₀/ml compared to vehicle-treated animals. We did not observe as much benefit from combining fluorines on b and c (AVG-435), but replacing the anisole moiety of AVG-233 with a 1,3 dichlorobenzene (AVG-445) resulted in a lung titer reduction of 1.1 (±0.16) log 10 TCID₅₀/ml, nearly as potent as that seen with AVG-388.

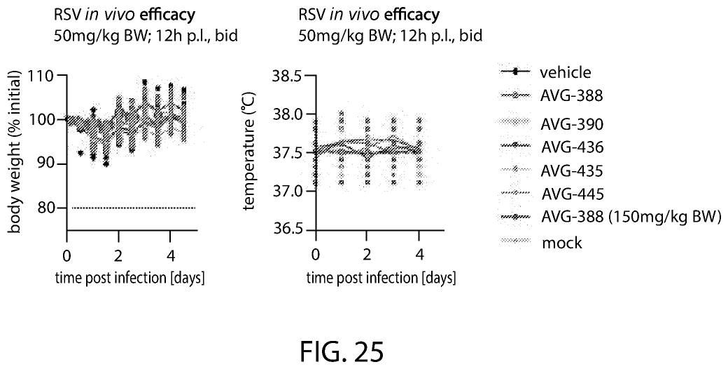

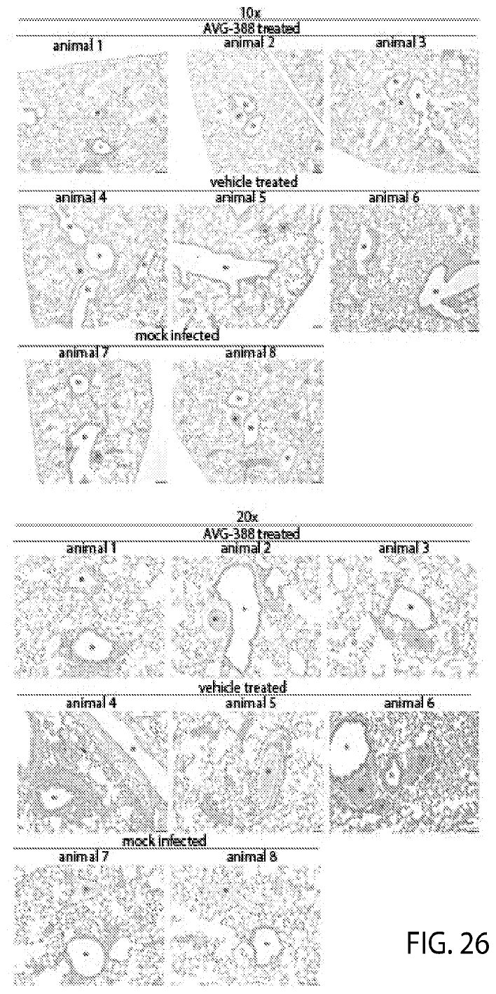

Antiviral efficacy of AVG-388 was dose-dependent, resulting in a strong reduction of 1.9 (±0.23) log 10 TCID₅₀/ml when dosed orally at 150 mg/kg b.i.d. (FIG. 6G) (Table 5) without any signs of adverse effects (FIG. 25). Histopathology analysis of lung tissue extracted 4 days after infection from vehicle-treated animals revealed prominent interstitial pneumonia with multifocal pleuritis and moderate perivascular lymphohistiocytic cuffing (FIG. 6H,1) (FIG. 26). Treatment with AVG-388 at 50 mg/kg initiated 12 hours after infection prevented disease progression to viral pneumonia. Only one animal of the AVG-388 group showed mild vasculitis and perivascular cuffing. These results demonstrating superior oral efficacy of AVG-388 against RSV beyond previous expectations based on the original compounds confirm successful synthetic optimization of the AVG-233 scaffold and identification of a developmental candidate for the treatment of RSV disease.

### Analysis

Our initial characterization of the AVG-233 class revealed that the inhibitor does not block phosphodiester bond formation per se, but disturbs initiation of viral RNA synthesis at the promoter (24). This inhibition pattern may reflect pharmacological interference with a predicted conformational rearrangement of the polymerase complex during initiation (43, 44). Three lines of experimental evidence support this view: the AVG class resistance profile, the MOA characterization in biochemical RdRP assays, and the photoaffinity labeling-based mapping of the target site.

The primary resistance hot-spot of the AVG class, L residue 1502, is positioned at the interface between the large RdRP domains mediating RNA synthesis and the MTase domain required for capping of nascent viral mRNAs. A secondary escape residue, L 1632, likewise locates to this junction, as does residue 1631, which is the primary resistance site for the experimental RSV inhibitors YM-53503 (25), AZ-27 (23), PC786 (26), and cpd1(27). Cross-resistance among chemically distinct antivirals with comparable MOA is not uncommon. However, distinct resistance profiles of mechanistically related chemotypes predicted to engage the same target domain such as AVG-233 and AZ-27 is rare, but opens interesting future possibilities for combination therapies. Divergent resistance profiles from mechanistically similar RSV L inhibitors indicate a unique docking pose of the AVG class.

Biochemical RdRP assays using different type of synthetic RNA templates demonstrated that compounds of the AVG class block de novo initiation of RNA synthesis and extension of a paired primer in a synthetic primer/template after the first few nucleotides. This initial delay does not represent an artifact of the RdRP assay, since we found previously that ERDRP-0519, a small-molecule inhibitor of measles virus polymerase that we have developed, completely blocks all phosphodiesterbond formation in the equivalent measles virus RdRP assay (45). Both AVG-233 inhibitory activities were sensitive to the L residue 1502 resistance mutation, indicating that suppressed de novo initiation and impaired RNA elongation are a consequence of a uniform AVG-233 docking pose to the L target. The apparent difference in AVG-233 EC₅₀ values between cell-based and in vitro RdRP assays likely reflects a high representation of bio-inactive L complexes in the P-L preparations, which is typical for purified mononegavirus polymerase complexes (46). However, other mechanisms are also conceivable. While absent in cellula, this unproductive material may absorb compound in the biochemical assay without appreciable inhibitory effect. Although less likely, the AVG-233 could alternatively affect transcription, since photo-crosslinking proposed a docking pose in which the compound could also interfere with L activities such as cap binding and/or cap methylation. We furthermore cannot fully exclude that AVG-233 may be metabolically modified by cellular enzymes, increasing its target affinity.

Beyond resistance profiling, no physical target site has been mapped for any of the experimental RSV RdRP inhibitors. We closed this knowledge gap for the AVG class, identifying L residues in direct proximity to the docked ligand that spanned an interface between the L capping, connecting, and MTase domains. Confidence in specificity and physiological relevance of the photoaffinity labels comes from three sources: three chemically distinct analogs were generated that differentially interrogate the target site; each of these analogs maintained potent anti-RSV activity; and residues covalently identified by these analogs lined a continuous physical site in the native polymerase complex.

Structural reconstructions of RSV (17, 18), closely related human metapneumovirus (47), and more distantly related paramyxovirus (48) L-P complexes have highlighted a dynamic organization of the C-terminal L domains relative to the polymerase core composed of RdRP and capping domains. Based on the mechanistic inhibition profile in the in vitro RdRP assays, the resistance data and the photoaffinity maps, we considered two mechanistic alternatives as possible molecular basis for AVG-233 inhibition of L: structural lock-down of the MTase domain or positional fixation of the putative priming-capping loop.

Specifically, AVG-233 docking may trap L capping, connecting, and MTase domains in a fixed position relative to each other, resulting in a polymerase complex that is permanently locked in initiation conformation. Preventing relative repositioning of these three domains should impair proper mRNA synthesis, since the MTase must swing away from the product exit channel after cap methylation to allow nascent mRNA elongation. However, our studies revealed that AVG-233 is active also against truncated L₁₋₁₇₄₉ RdRP complexes lacking the MTase domain, arguing against this model of MTase blockage of the exit channel.

We therefore favor the alternative explanation that AVG-233 prevents reorganization of the L priming-capping loop (49) after incorporation of the first few nucleotides. In RSV L structural models, the loop is retracted downstream of a pivotal residue G1264, which clears a path for the newly synthesized RNA strand to exit the polymerase complex in post-initiation configuration (17, 18). Residue G1264 is located in immediate spatial proximity to V1384, which photoaffinity mapping identified as a direct anchorpoint for AVG inhibitors, thus positing the docked ligand at the hinge region of the priming-capping loop. Although an actual role of the loop in priming of pneumovirus polymerases has not yet been formally proven (47), we note that a purified mutant RSV RdRP complex bearing a G1264A substitution was unable to elongate RNA beyond the addition of 2-3 nucleotides (50), thus mimicking the inhibition phenotype of AVG-233.

Despite predicted oral bioavailability in mice (24), in vivo efficacy of AVG-233 was under these test conditions was less than originally anticipated. Our targeted synthetic program identified a trifluoromethyl substitution in the c ring as instrumental for establishing robust oral efficacy. Since antiviral activity of AVG-233 in cell-based anti-RSV and the in vitro RdRP assays closely resembled that of the efficacious analogs, we hypothesize that the chloro-substituent in ring (c) of AVG-233 presented a metabolic liability in vivo that was overcome with the trifluoromethyl replacement.

Substantiated by a robust SI profile, potent antiviral performance in human tissue organoids and favorable pharmacokinetic properties, the orally efficacious AVG class leads have strong developmental potential. In addition to its immediate impact as a clinical candidate, the AVG chemotype has identified the interface between the RSV L capping, connecting and MTase domains as a major druggable site that is likely mechanistically conserved in all mononegavirus polymerase proteins. Considering the available resistance information, we propose that all allosteric RSV RdRP inhibitors interfering with polymerase initiation at the promoter that have been developed to date physically engage this interface. Our results lay the foundation for formal development of the AVG class and the structure-guided identification of companion drugs with overlapping target sites but distinct resistance profiles.

Further information regarding the specific testing protocols of this example are provided below.

### Materials and Methods/Experimental Design

In this study we explored the preclinical efficacy of a series of allosteric inhibitors of RSV RdRP both using the mice model and the disease-relevant differentiated primary cells from human bronchial/tracheal epithelium, and we determined the mechanism of action of this class in vitro. The efficacy models were chosen because they jointly provide the closest model available to RSV replication in human lungs, and constitute the premier system to evaluate efficacy of drug candidates. We determined the effect of treatment on viral replication at different oral doses in a prophylactic or therapeutic setting. Efficacy was considered as a statistically significant reduction in viral titers in mice lungs and in apical shedding from differentiated human epithelium. Efficacy and cytotoxicity in cell culture were determined in a dose-dependent manner using four-parameter variable-slope regression modeling and calculation of 50% maximal efficacious concentrations. The endpoints were predefined for each experiment. Animals were randomly assigned to each group. Numbers of independent biological repeats for all experiments are specified in the figure legends. No blinding was performed. Complete raw and analyzed numerical data were maintained.

### Cell Lines, Plasmids and Viruses

HEp-2 cells (ATCC® CCL-23™), HEK-293T (ATCC® CRL-3216™) and baby hamster kidney cells (BHK-21; ATCC® CCL-10™) stably expressing either T7 polymerase (BSR-T7/5) were grown at 37° C. and 5% CO₂ in Dulbecco's modified Eagle's medium (DMEM) supplemented with 7.5% heat-inactivated fetal bovine serum (FBS). Insect cells from Spodoptera Frugiperda (SF9, ATCC® CRL-1711™) were propagated in suspension using Sf-900 II serum-free media (SFM) (Thermo Scientific) at 28° C. without CO₂. Experiments with primary human airway epithelial cells involved tissues from the following donors: Primary human bronchial tracheal epithelial cells from a 30-year-old female (“F1” donor, LifeLine Cell Technology, cat #LM-0050, lot #3123, passage 2) and primary human normal bronchial/tracheal epithelial cells (NHBE) (“M4” donor, Lonza Bioscience, cat #CC-2540S, lot #0000646466, passage 2) from a 38-year-old male were cultured in BronchiaLife complete cell culture medium (LifeLine Cell Technology, cat #LL-0023). Differentiated human airway epithelium from “F1” donor (3D-HAE) was obtained as described previously (35) Briefly, 33,000 low passage (<4) were seeded in polyester, tissue culture treated, 0.33 cm² growth area, 0.4 μm membrane pore size Transwell (Corning, cat #3470). Basal media was replaced with Pneumacult-ALI (Stemcell Technologies cat #05001) when confluency was reached, and apical media was removed. Transepithelial/transendothelial electrical resistance (TEER) was monitored to validate differentiation with EVOM volt/ohm meter and STX2 electrode (World Precision Instruments). All cell lines used in this study were authenticated and checked for mycoplasma and microbial contamination. Note the International Cell Line Authentication Committee (ICLAC) lists HEp-2 cells as a commonly misidentified cell line, however their unique ability to efficiently propagate RSV justifies their use to generate and titrate viral stocks. Recombinant respiratory syncytial virus (RSV) strain A2 with line19F and mKatushka or fireSMASh reporter gene, recRSV-mKate or recRSV-fireSMASh respectively, was rescued and amplified as described previously(51, 52). L1502Q, H1632Q and Y1631H substitutions were introduced following a protocol described previously (5).

### Dose Response Antiviral Assays

Compounds stocks were prepared in dimethyl sulfoxide (DMSO) and upon dilution in cell culture media reached in all wells a final DMSO concentration of 0.1%. For luciferase-based dose-response assays, HEp-2 or primary HAE cells were seeded a day before to reach 50% confluence in 96-well white plates. 3-fold serial dilutions of compounds were prepared in triplicate using an automated Nimbus liquid handler (Hamilton) and transferred to the cells. Immediately after addition of compound, cells were infected with recRSV-fireSMASh. Each plate contained 4 wells each of positive and negative control (media containing 100 μM cycloheximide or vehicle, respectively). Luciferase activities were determined at 48 h post-transfection using One-GLO buffer (Promega, cat #E6130) and a Synergy H1 (BioTek) plate reader. Normalized luciferase activities were analyzed with the formula: % inhibition=(Signal_{Sample}−Signal_{Min})/(Signal_{Max}−Signal_{Min})×100, and dose response curves were further analyzed by normalized non-linear regression with variable slope to determine 50% effective concentration (EC₅₀) and 95% confidence intervals (Cis) with Prism 9.0.1 for MacOS (GraphPad).

### Minireplicon Assays

A set of helper plasmids expressing codon-optimized RSV P, L, N and M2-1 proteins (A2 strain) under the control of CMV promoter, and a plasmid expressing the RSV minigenome cassette containing the firefly luciferase reporter, expressed under control of RNA pol I promoter, were co-transfected with GeneJuice reagent (Millipore Sigma) following manufacturer's instructions in 50% confluent HEK-293T cells or BSR-T7/5 cells as described previously (5). To assay RdRP complexes inhibition in dose-response experiments, cells were transfected in 96-well white plates. At 4 hours post-transfection, compounds were added to the cells and analyzed as described above.

### Cytotoxicity Assays

HEp-2, or primary HAE cells were seeded at 50% confluence in 96-well plates and were incubated with 3-fold serial dilution of compound with positive and negative controls as described above. After 48-hour incubation at 37° C., cells were incubated with PrestoBlue (ThermoFisher Scientific, cat #A-13262) for 1 h at 37° C. and fluorescence measured with a H1 synergy plate reader (BioTek). 50% cytotoxic concentrations (CC₅₀) and 95% CIs after normalized non-linear regression and variable slope were determined using Prism 9.0.1 for MacOS (GraphPad).

### Growth Curves

HEp-2 cells seeded in 12-well plates were infected with recRSV-fireSMASh in absence or presence of L1502Q, H1632Q or Y1631H substitution in L, at multiplicity of infection of 0.1 in three independent replicates. Cells were scrapped and harvested every 12 hours for 4 days following infection. After virus release through freeze-thaw and clarification, viral titers were determined through TCID₅₀ titration with firefly luciferase bioluminescence as the readout, using One-GLO buffer (Promega, cat #E6130) and a Synergy H1 (BioTek) plate reader.

### Confocal Microscopy

3D-HAE were fixed for 30 minutes at room temperature with a solution of 4% paraformaldehyde diluted in PBS. After permeabilization and blocking with PBS+3% bovine serum albumin (BSA)+0.1% Triton X-100 (45 minutes), cells were incubated at room temperature for 1 hour with primary antibodies diluted in PBS+0.3% BSA+0.05% Tween20 (wash buffer). After three 5-minute washes, cells were incubated with a secondary antibody in wash buffer for 45 minutes, incubated with Hoechst 34580 (BD Biosciences, cat #565877) (1:1000) in wash buffer for 5 minutes. Membranes were mounted on glass slides with ProLong Diamond Antifade Reagent (ThermoFisher Scientific, Cat #P36970) and imaged with a Zeiss Axio Observer Z.1 and Zeiss LSM 800+AiryScan module. Image analyses were performed with Zeiss Zen 3.1 Blue software (Windows 10). Representative pictures were taken either with a 63× Plan. Apochromat. (NA: 1.40, oil) objective. Digital pictures are pseudocolored for optimal presentation. Acquisition of 35 μm depth with 0.22 μm slices unless stated otherwise in figure legend.

### Apical Shed Viral Titer Determination

3D-HAE were washed apically with PBS without calcium and magnesium. Cells were infected apically with recRSV-mKate (500,000 TCID₅₀) for 2 hours at 37° C. Compound was added in the basal media (vehicle: 0.1% final DMSO). Shed virus was harvested by incubating the apical side with 200 μl PBS without calcium and magnesium at 37° C. for 30 minutes. Aliquots were stored at −80° C. until titration by standard TCID₅₀ using fluorescence to detect infected cells.

### Antibodies

Antibodies used for viral titration (TCID₅₀) were RSV: Goat Anti-Respiratory Syncytial Virus Polyclonal Antibody (1:1000 dilution) (Millipore Sigma, cat #AB1128) followed by donkey anti-goat antibody conjugated with horseradish peroxidase (1:1000 dilution) (Jackson Immunoresearch, cat #705-035-147). Infected cells were detected using Trueblue peroxidase substrate according to the manufacturer's instructions (Fisher Scientific, cat #5067428).

Primary antibodies used for confocal microscopy: Adherens junctions were visualized with E-Cadherin mouse antibody (1:100 dilution) (BD Biosciences; 610181). Goblet cells were visualized with mouse anti-MUC5AC (1:200 dilution) (ThermoFisher, cat #MA5-12175). Ciliated cells were visualized with rabbit anti-beta IV tubulin recombinant antibody conjugated with Alexa Fluor® 647 [EPR16775] (1:100 dilution) (Abcam, cat #ab204034). Tight junctions were visualized with mouse anti ZO-1 (1:50 dilution) (BD Biosciences, cat #610966). RSV infected cells were visualized either as a whole with Goat Anti-Respiratory Syncytial Virus Polyclonal Antibody (1:1000 dilution) (Millipore Sigma, cat #AB1128), or with a focus on RSV-induced cytoplasmic inclusion bodies using mouse Anti-RSV nucleoprotein, clone 130-12H (1:100 dilution) (Millipore Sigma, cat #MAB858-3). The following antibodies were used as secondary antibodies as appropriate: rabbit anti-mouse IgG (H+L) cross-adsorbed secondary antibody, Alexa Fluor® 488 (1:500 dilution) (ThermoFisher Scientific, cat #A-11059) or Donkey anti-goat Alexa Fluor® 568 (1:500 dilution) (ThermoFisher Scientific, cat #A-11057).

### Protein Purification

RSV L+P complexes were prepared as previously described (53, 54). Briefly, codon-optimized sequences of RSV L and a 6×HIS-tagged P were co-expressed in SF9 cells in serum-free medium SF900-II (ThermoFisher Scientific) from a recombinant baculovirus vector generated with the pFastBac dual system. Cells were harvested at 78 h.p.i. and lysed in 50 mM NaH2PO4 [pH 8.0], 150 mM NaCl, 20 mM imidazole, 0.5% Igepal (Millipore-sigma). After purification through immobilized metal affinity chromatography with Ni-NTA Superflow resin (Qiagen), cells were dialyzed into storage buffer: 20 mM Tris-HCl [pH 7.4], 150 mM NaCl, 10% glycerol, 1 mM dithiothreitol.

### In Vitro Polymerase Assay

de novo RNA synthesis using purified L+P complex was performed as previously described(54). Briefly, 100-200 ng of RSV L in complex with P were incubated with 2 μM RNA template corresponding to the 25 nt of the RSV trailer complement sequence (3′ UGCUCUUUUUUUCACAGUUUUUGAU) (Horizon Discovery), 8 mM MgCl₂, 1 mM dithiothreitol, 1 mM each of ATP, UTP, CTP, 50 μM GTP, 10 μCi of (alpha)³²P-labelled GTP (Perkin-Elmer), 20 mM Tris-HCl [pH 7.4], 15 mM NaCl, 10% glycerol. Reaction were equilibrated 10 minutes at 30° C. before addition of L+P complexes, then incubated for 3 hrs at 30° C. RNAs were precipitated for 16 hrs at −20° C. with 2.5 volumes of ice-cold ethanol, 0.1 volume of 3M sodium acetate and 625 ng of Glycogen (ThermoFisher Scientific). Pellets were washed with ice-cold 75% ethanol, dried and resuspended in 50% deionized formamide. After a 3-minute denaturation at 95° C., RNAs were separated on 7M urea 20% polyacrylamide Tris Borate-EDTA gels and visualized by autoradiography using either CL-XPosure™ Film (ThermoFisher Scientific) or a storage phosphor screen BAS IP MS 2040 E (GE Healthcare Life Sciences) and imaged with Typhoon FLA 7000 (GE Healthcare Life Sciences). Densitometry analysis was performed using Fiji 2.0 (55).

3′ extension assays were performed based on slight modifications of established assays(46, 56, 57). Briefly, 1 μM RNA template (3′ UGGUCUUUUUUGUUUC) and 200 μM of 5′ phosphorylated RNA primer (5′ pACCA) (Horizon Discovery) were incubated with 8 mM MgCl₂, 1 mM dithiothreitol, 10 μM each of ATP, UTP, CTP, GTP and 10 μCi of (alpha)³²P-labelled GTP (Perkin-Elmer), 20 mM Tris-HCl [pH 7.4], 15 mM NaCL, 10% glycerol, and after 10 minutes at 30° C. with 100 ng RSV L in complex with P in a final volume of 5 μl. After 60 minutes incubation at 30 C, reaction was stopped with 5 μl of deionized formamide with 25 mM Ethylenediaminetetraacetic acid (EDTA). After denaturation at 95° C., RNAs were separated on 7M urea 20% polyacrylamide Tris Borate-EDTA gels and visualized by autoradiography using a storage phosphor screen BAS IP MS 2040 E (GE Healthcare Life Sciences) and imaged with Typhoon FLA 7000 (GE Healthcare Life Sciences). Densitometry analysis was performed using Fiji 2.0 (55).

### Biolayer Interferometry

Purified RSV L-P complexes were buffer-exchanged for phosphate-buffered saline (PBS) pH 7.4 [RT] on PD-10 desalting columns (GE healthcare), mono-biotinylated with the EZ-Link™ Sulfo-NHS—SS-Biotin reagent (ThermoFisher Scientific) and loaded on Super-Streptavidin sensors (Molecular Devices) for 2 hrs at 30° C. to reach a shift of 1 nm. Uncoupled streptavidin was quenched for 15 minutes with a solution of 2 mM biocytin. In parallel, a 1 mg/ml solution of Thyroglobulin (GE healthcare) was biotinylated and loaded to 1 nm shift on control sensors. Kinetic experiments were performed at 30° C. with 1000 rpm shaking in 96 well plates using the Octet Red 96 system (Fortebio). Biosensors loaded with L-P and Thyroglobulin were successively equilibrated for 100 s in assay buffer [PBS, BSA 0.01%, Tween-20 0.005%, DMSO 1%] [baseline], incubated in a dilution of compound [2-fold from 40 nM to 40 μM] for 120 s [association], then incubated in assay buffer for 200 s [dissociation]. Real-time binding kinetics were analyzed and calculated using the Octet Red software package. Raw signal was processed using the double reference method, by subtracting both the thyroglobulin signal (unspecific signal) and the signal in absence of compound (drift), after baseline-alignment and inter-step correction at the dissociation. Kinetic modelling was done by analyzing association and dissociation signals using Global fitting with a 1:1 model.

### Photolabeling of the AVG-233 Binding Site

2 μg of RSV L-P complexes in PBS were mixes with 100 μM of compound a, b or c for 5 minutes on ice, then photo-crosslinked for 10 minutes (compound a) or 45 minutes (compound b and c) at 365 nm. Samples with compounds b and c were further treated with the auto-crosslink mode of the Stratalinker 1800 (Stratagene). Samples were fractionated on Bolt™ 4-12% Bis-Tris Plus Gels (ThermoFisher Scientific) and MES buffer, and analyzed by mass spectrometry.

### LC-MS/MS Analyses and Data Processing

Liquid chromatography tandem mass spectrometry (LC-MS/MS) analysis was performed by the Proteomics and Metabolomics Facility at the Wistar Institute using a Q Exactive Plus mass spectrometer (ThermoFisher Scientific) coupled with a Nano-ACQUITY UPLC system (Waters). Following procedures originally described in (45) (Cox et al., PLoS Pathog. 17, e1009371 (2021), said article incorpoirated herein by reference), gel bands were excised, digested in-gel with trypsin and injected onto a UPLC Symmetry trap column (180 μm i.d.×2 cm packed with 5 μm C18 resin; Waters). Tryptic peptides were separated by reversed phase HPLC on a BEH C18 nanocapillary analytical column (75 μm i.d.×25 cm, 1.7 μm particle size; Waters) using a gradient formed by solvent A (0.1% formic acid in water) and solvent B (0.1% formic acid in acetonitrile). A 30-minute blank gradient was run between sample injections to minimize carryover. Eluted peptides were analyzed by the mass spectrometer set to repetitively scan m/z from 300 to 2000 in positive ion mode. The full MS scan was collected at 70,000 resolution followed by data-dependent MS/MS scans at 17,500 resolution on the 20 most abundant ions exceeding a minimum threshold of 20,000. Peptide match was set as preferred, exclude isotopes option and charge-state screening were enabled to reject unassigned charged ions. Peptide sequences were identified using pFind 3.1.5 (58). MS/MS spectra were searched against a custom database containing Sf9, Baculovirus, and hRSV protein sequences. Search parameters include full tryptic specificity with up to three missed cleavages, peptide mass tolerance of 10 ppm, fragment ion mass tolerance of 15 ppm, static carboxamidomethylation of Cys, and variable oxidation of Met. In addition, mass addition of 537.117924 (compound a), 470.125788 (compound b) or 484.165901 (compound c) was also considered for all amino acid residues. Consensus identification lists were generated with false discovery rates of 1% at protein and peptide levels.

### Molecular Modelling and Docking

RSV L-P available structure encompasses residue 1-1460 of RSV, likely in a post-initiation conformation. (18) (PDB: 6PZK). Modelling of full-length RSV L in putative pre-initiation conformation was performed using I-Tasser server and the RSV L strain A2 (GenBank: AAC14905.1) using as a template related vesicular stomatitis virus L (PDB: 5a22), L+P (PDB: 6U1X) Rabies virus L+P (PDB: 6UEB) and human metapneumovirus L C-terminal domain (PDB: 4UCY).

### In Vivo Efficacy Testing

Female Balb/cJ mice (Jackson laboratory, cat #000651) 6-8 weeks of age were housed in an ABSL-2 facility (4-5 day rest). Mice were divided randomly into groups of 5, and infected intranasally with 500,000 TCID₅₀ (25 μl/nare) of recRSV-mKate in Phosphate-Buffered Saline. Mice were anesthetized with ketamine/xylazine. Treatments were administrated via oral gavage in a 200 μl suspension of 1% Methylcellulose in water. Temperature and body weight were determined on a daily and twice-daily basis, respectively. Efficacy studies were terminated at peak viral replication at 4.5 days post-infection, mice were euthanized and lungs harvested and weighted. Lung viral titers were determined after tissue homogenization with a bead beater with 300 μl PBS (3 bursts of 30 seconds at 4° C., separated by 30-second rest at 4° C.). Homogenates were clarified (5 minutes at 4° C. and 20,000×g), aliquoted and stored at −80° C. until titration. Viral titers were determined by median tissue culture infectious dose (TCID₅₀) titration, adjusted to weight (g) of lung tissue.

### Histopathology Analysis

For histopathology, mice were subjected to cervical dislocation 4.5 days after infection and lungs perfused with 10% NBF prior to extraction. Lungs were stored in 10% NBF for 24 hours, followed by incubation in 70% EtOH for two days and embedding into wax blocks, using a 21-hour alcohol-xylene-wax embedding sequence. Blocks were sectioned at ˜4 μm thickness, sections mounted onto microscopy slides, and stained with hematoxylin and eosin (H&E). Slides were examined by a board-certified veterinary pathologist, who was blinded to the treatment groups. Lesions were scored according to the following scale. Alveoli, bronchiolar, and pleuritis scores: 1=focal, 2=multifocal, 3=multifocal to coalescing; perivascular cuffing: 1=1 layer of leukocytes cuffing vessel, 2=2-5 layers, 3=6-9 layers; vasculitis score: 1=leukocytes infiltrating vessel wall, 2=leukocytes and smooth muscle cell separation, 3=fibrinoid necrosis; interstitial pneumonia: 1=alveolar septa infiltrated by 1 leukocyte thickness, 2=2 leukocytes thick, 3=3 leukocytes thick.

### Chemical Synthesis

All materials were obtained from commercial suppliers and used without purification, unless otherwise noted. Dry organic solvents, packaged under nitrogen in septum sealed bottles, were purchased from EMD Millipore and Sigma-Aldrich Co. Reactions were monitored using EMD silica gel 60 F254 TLC plates or using an Agilent 1200 series LCMS system with a diode array detector and an Agilent 6120 quadrupole MS detector. Compound purification was accomplished by liquid chromatography on a Teledyne Isco CombiFlash RF+flash chromatography system. NMR spectra were recorded on an Agilent NMR spectrometer (400 MHz) at room temperature. Chemical shifts are reported in ppm relative to residual solvent signal. The residual shifts were taken as internal references and reported in parts per million (ppm).

### General Reaction Strategy:

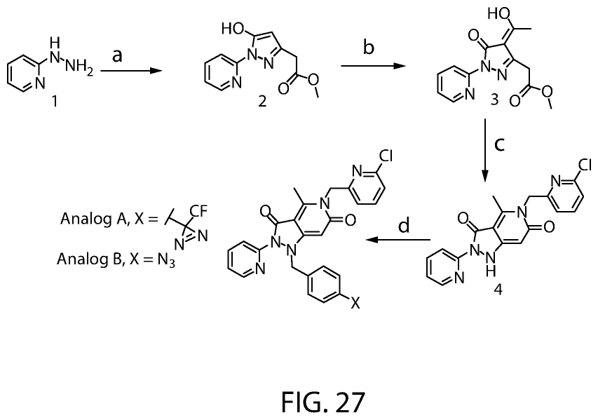

An overview of the general synthesis strategy of the AVG chemotype is shown in FIG. 27. Dimethyl-1,3-acetonedicarboxylate (6.6 ml, 45.81 mmol) was added to a suspension of 2-hydrazinopyridine (compound 1; FIG. 27) (5.0 g, 45.81 mmol) in anhydrous toluene (50 ml) to synthesize compound 2 (FIG. 27). The reaction mixture was refluxed for 12 hours. After completion, the reaction mixture was concentrated under reduced pressure. The brown solid product (9.60 g, 90% yield) obtained was washed with diethyl ether and dried in vacuum oven at 50° C. and used as such for further reactions.

1H NMR 400 MHz, DMSO-d₆, δ 12.45 (s, 1H), 8.43 (d, J=8 Hz, 1H), 8.02 (pseudo t, J=8 Hz, 1H), 7.74 (d, J=8 Hz, 1H), 7.35 (pseudo t, J=8 Hz, 1H), 5.58 (s, 1H), 3.64 (s, 3H), 3.61 (s, 2H). 13C NMR 100 MHz, DMSO-d₆, δ 170.67, 155.88, 153.23, 147.70, 146.90, 140.83, 121.25, 112.97, 88.68, 52.33, 34.99. MS (ES-API) [M+1]⁺:234.0

For synthesis of compound 3 (FIG. 27), triethyl orthoacetate (11.8 ml, 64.32 mmol) and acetic acid (0.25 ml, 4.30 mmol) were added to a solution of compound 2 (5.0 g, 21.43 mmol) in anhydrous acetonitrile (50 ml). The reaction mixture was stirred at 70° C. for 12 hours. After completion, the reaction mixture was concentrated under reduced pressure and washed with diethyl ether to obtain compound 3 as brownish yellow solid. 1H NMR 400 MHz, DMSO-d₆, δ 14.95 (broad s, 1H), 8.43 (d, J=8 Hz, 1H), 8.26-8.16 (m, 2H), 7.42-7.40 (m, 1H), 3.78 (s, 2H), 3.60 (s, 3H), 2.31 (s, 3H); ¹³C NMR 100 MHz, DMSO-d₆, δ 190.38, 169.41, 163.57, 150.71, 148.11, 143.64, 141.81, 119.75, 112.63, 102.78, 51.70, 34.78, 28.11. MS (ES-API) [M+1]⁺:276.0.

For synthesis of compound 4 (FIG. 27), (6-Chloropyridin-2-yl)methanamine dihydrochloride (0.64 g, 3 mmol) and triethylamine (1.25 ml, 9 mmol) were added to a solution of compound 3 (0.8 g, 3 mmol) in anhydrous acetonitrile (20 ml), and the reaction mixture was stirred at room temperature. After 2 hours, DBU (0.9 ml, 6 mmol) was added and stirred at room temperature for another 5 hours. After completion, the reaction mixture was concentrated under reduced pressure and the crude product was purified by flash column chromatography using dichloromethane and methanol as eluent. Product was obtained as yellow solid (0.40 g, 36% yield). 1H NMR 400 MHz, DMSO-d₆, δ 11.48 (s, 1H), 8.48-8.46 (m, 1H), 8.37 (d, J=8 Hz, 1H), 7.95-7.91 (m, 1H), 7.84 (pseudo t, J=8 Hz, 1H), 7.42 (d, J=8 Hz, 1H), 7.28-7.22 (m, 2H), 5.68 (s, 1H), 5.37 (s, 2H), 2.83 (s, 3H); ¹³C NMR 100 MHz, DMSO-d₆, 190.38, 169.41, 163.57, 150.71, 148.11, 143.64, 141.87, 119.77, 112.62, 109.58, 102.79, 51.65, 28.11. MS (ES-API) [M+1]⁺:368.0.

For synthesis of analogs A and B (FIG. 27), N,N-diisopropylethylamine (0.10 ml, 0.60 mmol) and respective substituted benzyl halide (0.60 mmol) were added to a solution of compound 4 (0.18 g, 0.5 mmol) in anhydrous N,N-Dimethylformamide (5 ml), and the reaction mixture was stirred at 50° C. for 6 hours. After completion, the reaction mixture was concentrated under reduced pressure and the crude product was purified by flash column chromatography using dichloromethane and methanol as eluent. Desired products were obtained in 25 to 35% isolated yield.

Analog A: 1H NMR 400 MHz, CDCl₃, δ 8.54 (d, J=8 Hz, 1H) 7.85-7.78 (m, 2H), 7.61 (pseudo t, J=8 Hz, 1H), 7.26-7.16 (m, 4H), 7.06-6.99 (m, 3H), 6.06 (s, 1H), 5.40 (s, 2H), 4.98 (s, 2H), 2.89 (s, 3H); 19F NMR 376 MHz, CDCl₃, δ □65.08 (s); 13C NMR 100 MHz, CDCl₃ (includes multiplets from 13C-19F couplings), ≤162.91, 162.16, 156.01, 155.20, 154.40, 151.01, 148.65, 148.31, 148.21, 139.62, 138.25, 135.33, 129.31, 129.25, 129.16, 129.09, 126.62, 126.53, 126.45, 123.53, 123.38, 123.27, 121.08, 120.93, 120.64, 120.54, 120.49, 117.17, 101.66, 90.88, 90.71, 77.09, 53.82, 48.14, 15.33. MS (ES-API) [M+1]⁺:565.9.

Analog B: 1H NMR 400 MHz, CDCl₃, δ 8.54 (d, J=8 Hz, 1H), 7.85 (m, 2H), 7.61 (pseudo t, J=8 Hz, 1H), 7.26-7.16 (m, 3H), 7.06-6.99 (m, 4H), 6.06 (s, 1H), 5.40 (s, 2H), 4.98 (s, 2H), 2.89 (s, 3H); 13C NMR 100 MHz, CDCl₃, δ 162.95, 162.16, 156.09, 155.31, 154.22, 151.01, 148.68, 148.25, 140.28, 139.64, 138.24, 130.44, 130.31, 130.12, 123.38, 120.89, 120.64, 120.49, 119.15, 117.19, 101.90, 91.02, 53.91, 48.17, 15.31. MS (ES-API) [M+1]⁺:498.9.

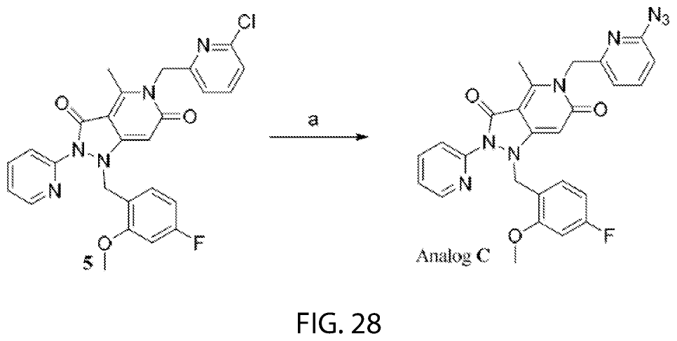

An overview of the preparation of analog C is shown in FIG. 28. Sodium azide (0.32 g. 5.0 mmol) and ammonium chloride (0.26 g, 5 mmol) were added to a solution of compound 5 (0.25 g, 0.5 mmol; FIG. 28, prepared as shown in FIG. 27 for the synthesis of analogs A and B) in anhydrous DMF (3 ml), and stirred at 110° C. for 48 hours. After completion, the reaction mixture was concentrated under reduced pressure. The crude reaction mixture was dissolved in dichloromethane (50 ml) and extracted with water (50 ml) and brine (50 ml). The organic layer was dried over anhydrous sodium sulfate and concentrated under reduced pressure. The crude product was purified by flash column chromatography using dichloromethane and methanol as eluent. Product was isolated as pale yellow solid (0.093 g, 36% yield). 1H NMR 400 MHz, CDCl₃, δ 8.56 (ddd, J=5.2 Hz, 2 Hz, 0.8 Hz, 1H), 8.01 (dd, J=8 Hz, 0.8 Hz, 1H), 7.84-7.80 (m, 1H), 7.72-7.70 (m, 1H), 7.61 (dd, J=12 Hz, 8 Hz, 1H), 7.22 (ddd, J=5.2 Hz, 2 Hz, 0.8 Hz, 1H), 6.88 (dd, J=8.4 Hz, 6.4 Hz, 1H), 6.76-6.74 (m, 1H), 6.50-6.41 (m, 2H), 6.18 (s, 1H), 5.95 (s, 2H), 5.04 (s, 2H), 3.50 (s, 3H), 2.84 (s, 3H); 19F NMR 376 MHz, CDCl₃, δ □109.51 to □109.57 (m); ¹³C NMR 100 MHz, CDCl₃ (includes multiplets from 13C-19F couplings) δ 164.90, 162.67, 162.44, 162.10, 158.86, 155.12, 152.77, 148.94, 148.76, 148.17, 137.97, 134.09, 131.88, 120.96, 117.73, 117.40, 114.78, 113.43, 106.79, 106.58, 102.59, 99.03, 98.78, 90.84, 55.31, 48.44, 42.44, 14.86; MS (ES-API) [M+1]⁺:513.0.

### Statistical Analysis

GraphPad Prism software (v8.3.0 Mac OSX) was used for all statistical analyses. Multiple comparisons were analyzed with One-way or two-way ANOVA with Dunnett's or Sidak's post hoc tests, as specified in figure legends. 50% and 90% maximal effective concentration were calculated using four-parameter non-linear regression modelling. Individual biological replicates (n=3) are represented as symbols or means with standard deviations. Significance threshold was set to 0.05. P values are represented as stars (*: p<0.05, **: p<0.01, ***: p<0.005, ****: p<0.001).

### DESCRIPTION OF THE FIGURES IN THIS EXAMPLE

The description of the figures referenced to above is included as follows:

FIG. 2. Resistance and mechanistic profiling of AVG-233. (A) Chemical structure of AVG-158, AVG-233 and AZ-27. (B) Schematic of the escalating-dose viral adaptation of recRSV-mKate with AVG-158 or AVG-233. (C) Schematics of RSV L with candidate resistance sites for AVG-233, AVG-158 and a reported resistance site to polymerase inhibitor AZ-27. (D,E) Dose-response inhibition of AVG-233 (D) and AZ-27 (E) against recRSV-mKate harboring L1502Q, Y1631H, or H1632Q substitution. Insets show EC₉₀ fold-changes relative to L WT. Highest AZ-27 concentration tested 6 μM; dotted area and star based on reported values (33). (F-J) in vitro RSV RdRP assays using synthetic primer/template pairs (G-J) or promoter sequence (1). Representative autoradiogram (G) with densitometric analysis (H). (I) relative in vitro RNA elongation in the presence of indicated doses of AVG-233 compared to vehicle-treated, in presence of L1502Q, Y1631H, or H1632Q substitution. (J) Densitometric quantitation of in vitro RNA elongation at each incorporated position in presence of indicated concentration of AVG-233. In all panels, symbols represent independent biological repeats (n=3), bars represent means, line represent 4-parameter variable slope regression modeling. Two-way ANOVA with Dunnett's post hoc tests (1,J).

FIG. 3. Label-free positive target identification of AVG-233. (A) Schematic of two RSV L targets examined, representing the full-length polypeptide (top) and a folding-competent (38) truncated L₁₋₁₇₄₉ polypeptide lacking the MTase and C-terminal (CTD) domains. (B) Purification of polymerase complexes specified in (A) after expression in insect cells. Coomassie-blue stain after SDS-PAGE fractionation. (C-D) Dose-dependent BLI-based association (120 seconds) and dissociation (200 seconds) curves (left) of AVG-233 with full-length RSV L (C) and L1-1749 (D), with non-linear fit with one-site specific binding (right). (E) In vitro RdRP assay using a synthetic promoter RNA template for de novo RNA synthesis, representative autoradiogram. (F—H) Primer/template-based in vitro RNA elongation assay, assessing bioactivity and AVG-233 inhibition of L[1-1749], with representative autoradiogram (F), densitometric quantitation of elongation products (G), and relative quantitation of each RNA products (H). Symbols represent independent biological repeats (n=3), bars represent means. Two-way ANOVA with Dunnett's post hoc tests.

FIG. 4. AVG-233 target site mapping through photo-affinity labeling. (A) Chemical structures of the AVG-233 analogs synthesized. Photo-activatable groups are highlighted in colored boxes. (B) Dose-response curves of the AVG-233 photoreactive analogs and standard AVG-233 against recRSV-fireSMASh. Symbols represent independent repeats (n=3). 4-parameter variable slope regression modeling. (C) RSV L-P peptides in close proximity of bound AVG-233, identified through photo-affinity labeling and LC-MS/MS analysis. Specific residue(s) engaged (crosslink localization) and confidence (PSM score) are shown. (D) Schematic representation of the three sets of peptides identified through each photo-activatable AVG-233 analog. (E) Cartoon representation of a structural model of the RSV P-L complex in putative pre-initiation state, based on RSV P-L reconstruction (PDB 6PZK) with residues (1461-2165) modeled after VSV P-L (PDB 6U1X). Color-coding as in FIG. 1. Photo-crosslinking target peptides from (C) and resistances mutations from FIG. 1 are highlighted. (F) Molecular docking of AVG-233 into proximity of photo-crosslinking targets and resistance sites L1502 and H1632. Residues involved in RNA synthesis (H1338 and R1339) and the putative priming loop are highlighted. (G) Predicted priming loop positions in postulated polymerase initiation and elongation conformations. Labeled are residues identified through photo-crosslinking and the adjacent, predicted priming loop pivot residue G1264.

FIG. 5. Efficacy of AVG-233 in well-differentiated human airway epithelial (3D-HAE) cells grown at air-liquid interface. (A) Schematics of 3D-HAE. (B) Confocal imaging of 3D-HAE. Tight junctions immunodetected with anti-ZO-1 (white). mucus producing goblet cells immunodetected with anti-Muc5AC (green). Ciliated cells immunodetected with anti-beta-tubulin (pink). Nuclei stained with Hoechst 35443 (blue). Scale bar 20 μm. (C) Multi-step growth curve of recRSV-mKate in 3D-HAEs. Viral titers were assayed from the apical chambers. Symbols show biological repeats (n=3), curve connects means±SD. (D) immunolabeling of recRSV-fireSMASh induced inclusion bodies with specific anti-RSV N antibody. Scale bar 2 μm. (E) Transepithelial electrical resistance of 3D-HAEs exposed basolaterally to 200 μM AVG-233 or vehicle (DMSO) for up to three days. Symbols represent means+/−SD (n=3). 2-way ANOVA with Sidak Post-hoc test. (F) Immunostaining of RSV N in RSV-infected cells with or without AVG-233 5 μM at 3 d.p.i. (G) Immunostaining of tight junctions in RSV-infected cells with AVG-233 5 μM at 3 d.p.i. (H) AVG-233 and NHC virus yield reduction against recRSV-mKate in 3D-HAEs. Progeny virus titers were determined six days after infection; symbols show biological repeats (n=3); curve connects mean values. EC₅₀ calculation through 4-parameter variable slope regression modeling.

FIG. 6. Identification of orally efficacious developmental analogs of AVG-233. (A-B), Effect of AVG-233 given orally twice daily at 12 h post-infection (A) to RSV-infected Balb/c mice on lung titers at 4.5 d.p.i. (B). (C) chemical structures of AVG-233 analogs with fluoro- or ortho-chloropyridine groups. (D-E) Comparison of AVG fluorine and ortho-chloropyridine analogs on antiviral potency in cell culture against recRSV-mKate (D) and in in vitro primer extension RdRP assays (E). (F-G) Effect of AVG-233 analogs given orally twice daily at 10 hours post-infection and 50 mg/kg (F) or 50 mg/kg and 150 mg/kg (G; AVG-388 only) to recRSV-mKate infected Balb/c mice on lung titers at 4.5 d.p.i. Symbols show individual biological repeats (independent experiments or individual animals), columns represent sample means. Unpaired t-test (B) or one-way ANOVA (F-G) with Dunnett's multiple comparisons post-hoc test; P values are shown.

FIG. 7. Dose-response inhibition of RSV minireplicon in presence of resistance mutation candidates. Values are normalized for vehicle-treated reactions; symbols represent individual biological repeats (n=3), determined in nine technical repeats each. EC₅₀ values and 95% confidence intervals are derived from 4-parameter variable slope regression models (solid line). Single or double black arrows visualize moderate (EC₅₀ fold change <10) or robust (EC₅₀ fold change >10) resistance, respectively.

FIG. 8. Multi-step growth curves of recRSV-fireSMASh harboring individual resistance mutations L1502Q, Y1631H, or H1632Q. Symbols represent independent biological repeats and lines connect medians. 2-way ANOVA with Dunnett's post-hoc test.

FIG. 9. Purified recombinant RSV RdRP (P-L) with resistance mutations or mutation N812A eliminating polymerase activity (59). Coomassie blue staining after SDS-PAGE fractionation; material representing L and P polypeptides is highlighted.

FIG. 10. Representative autoradiogram of primer extension assay from FIG. 21.

FIG. 11. Side-by side comparison of AVG-233 and AZ-27 in de novo RNA synthesis assay using L preparations harboring distinct resistance mutations. Color-coding of L preparations as in FIG. 1D-E.

FIG. 12. In vitro RdRP assay. The assay was performed as in FIG. 1I, using the alternative primer/template pair shown.

FIG. 13. Effect of endogenous nucleotides on AVG-233 RdRP inhibition. recRSV-fireSMASh-infected cells were treated with 20 μM of AVG-233 (left) or 10 μM of 4′-FIU (right) and serial dilutions of exogenous nucleosides were added to the extracellular media. Viral replication was determined by reporter activity and normalized for replication in the presence of vehicle (DMSO) volume equivalents instead of AVG-233 or 4′-FIU. Symbols represent independent repeats (N=3).

FIG. 14. Immunostaining of 3D-HAE. Tight junctions were detected with anti-ZO—I antibody (white). Cells were mock-infected or infected with recRSV-fireSMASh and treated with vehicle (0.1% DMSO) or AVG-233 or AVG-388 at 5 μM. Nuclei were stained with Hoechst 35443 (blue). Cultures were fixed and stained 3 days post-infection; scale bar 20 μm.

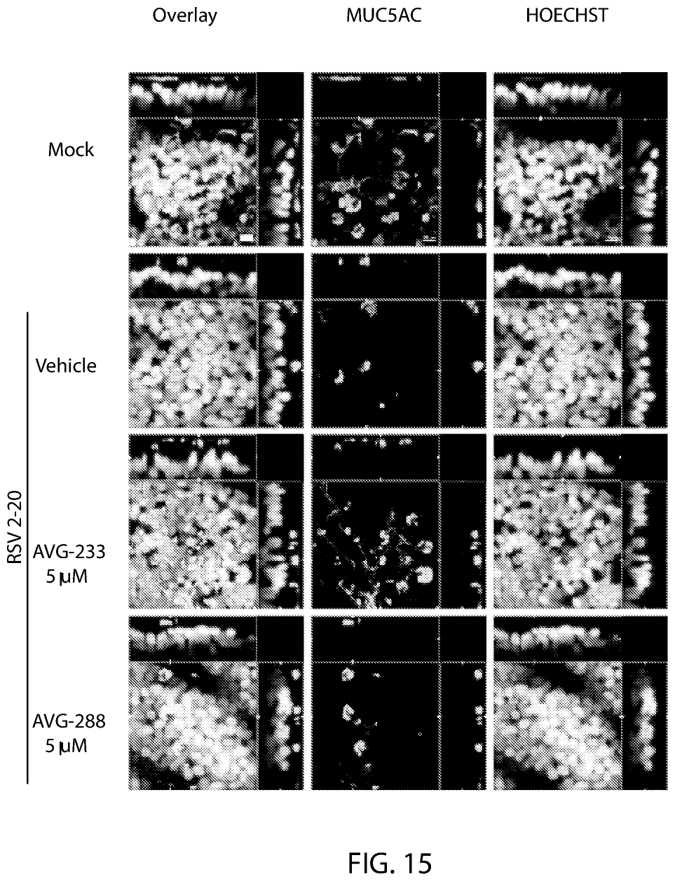

FIG. 15. Immunolabelling of 3D-HAE. Goblet cells were detected with anti-MUC5AC antibody (green). Cells were mock-infected or infected with recRSV-fireSMASh and treated with vehicle (0.1% DMSO) or AVG-233 or AVG-388 at 5 μM. Nuclei staining with Hoechst 35443 (blue). Cultures were fixed and stained 3 days post-infection; scale bar 20 μm.

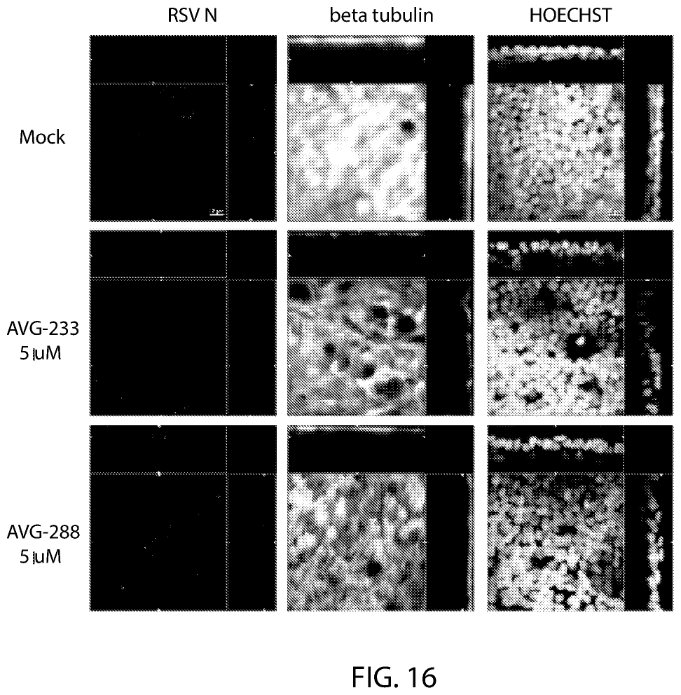

FIG. 16. Immunolabelling of 3D-HAE. Ciliated cells were detected with anti-beta-tubulin antibody (pink) and RSV-induced cytoplasmic inclusion bodies were detected with anti-RSV N (yellow). Cells were mock-infected or infected with recRSV-fireSMASh and treated with vehicle (0.1% DMSO) or AVG-233 or AVG-388 at 5 μM. Nuclei were stained with Hoechst 35443 (blue). Cultures were fixed and stained 3 days post-infection; scale bar 20 μm.

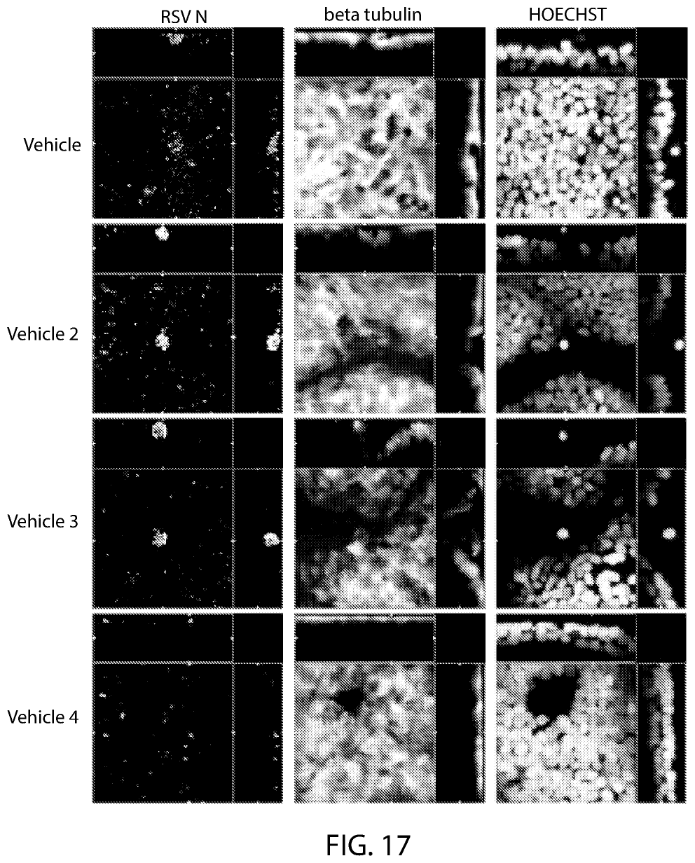

FIG. 17. Immunolabelling of 3D-HAE. Ciliated cells were detected with anti-beta-tubulin antibody (pink) and RSV-induced cytoplasmic inclusion bodies with anti-RSV N (yellow). Cells were mock-infected or infected with recRSV-fireSMASh and treated with vehicle (0.1% DMSO) or AVG-233 or AVG-388 at 5 μM. Nuclei were stained with Hoechst 35443 (blue). Cultures were fixed and stained 3 days post-infection; scale bar 20 μm.

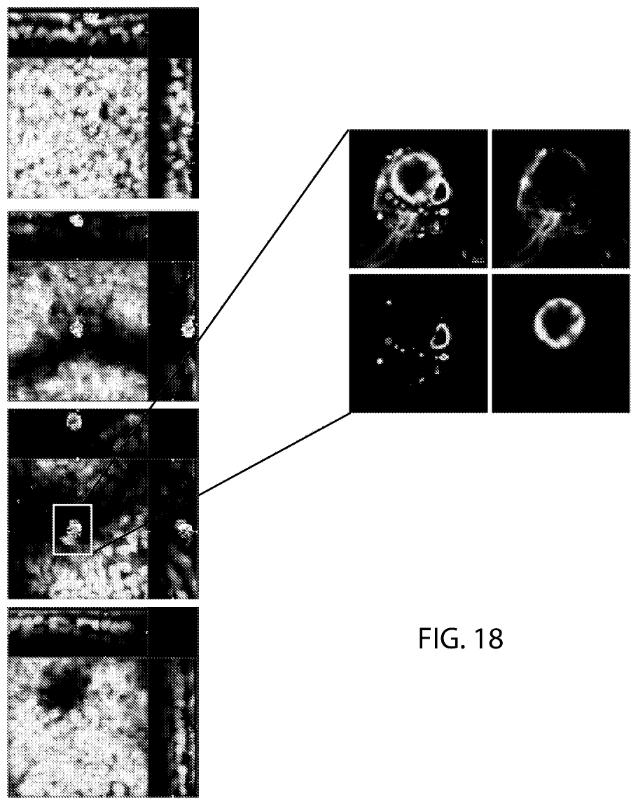

FIG. 18. Immunolabelling of 3D-HAE. Ciliated cells were detected with anti-beta-tubulin antibody (pink) and RSV-induced cytoplasmic inclusion bodies were detected with anti-RSV N (yellow). Cells were mock-infected or infected with recRSV-fireSMASh and treated with vehicle (0.1% DMSO) or AVG-233 or AVG-388 at 5 μM. Nuclei were stained with Hoechst 35443 (blue). Cultures were fixed and stained 3 days post-infection; scale bar 20 μm.

FIG. 19. Ciliated cells from 3D-HAE infected with recRSV-fireSMASh. RSV-infected cells were detected with a polyclonal anti-RSV antibody (red), mucus producing goblet cells were detected with specific anti-Muc5AC antibody (yellow), and nuclei were stained with DAPI (blue). Cultures were fixed and stained 10 days post-infection; scale bar 10 μm.

FIG. 20. Treatment with AVG-233 of 3D-HAEs infected with recRSV-fireSMASh. Adherens junction immunostaining is colored in yellow (anti-E-Cadherin), recRSV-fireSMASh infected cells immunostaining is colored in red (anti-RSV) and nucleus staining is colored in blue (DAPI); scale bar: 20 μm.

FIG. 21. Dose-response inhibition of recRSV-fireSMASh by analogs of AVG-233 in undifferentiated primary human airway epithelial cells. Top: “M4” donor, Bottom: “F1” donor. Values are normalized for vehicle-treated reactions; symbols represent individual biological repeats (n=3). EC₅₀ values and 95% confidence intervals (shown in Table 3) are derived from 4-parameter variable slope regression models (solid line).

FIG. 22. Dose-response inhibition of in vitro RdRP primer extension by analogs of AVG-233. Representative autoradiograms (n=3).

FIG. 23. Side-by-side comparison of AVG-233 and AVG-388 dose-response inhibition of either RSV minireplicon (top) and recRSV-fireSMASh (bottom). Minireplicon assays were performed either in HEK-293T cells or BSR-T7/5 cells. Values are normalized for vehicle-treated reactions; symbols represent individual biological repeats (n=3). EC₅₀ values and 95% confidence intervals are derived from 4-parameter variable slope regression models (solid line).

FIG. 24. Comparison of AVG-233 and AVG-388 cytotoxicity. Dose-response assays; Symbols represent means of individual biological repeats ±SD.

FIG. 25. Mouse bodyweight and temperature. Clinical signs of animals from the efficacy studies shown in FIG. 5F-I. Animals were treated orally with 50 mg/kg b.i.d. or 150 mg/kg b.i.d. (AVG-388 high dose only), and body weight and temperature determined. Symbols represent individual biological repeats (individual animals), lines connect group means.

FIG. 26. Lung histopathology. Photomicrographs of lung sections extracted 4.5 days after infection of animals and subjected to H&E staining, shown at 10× (top; scale bar 100 μm) and 20× (bottom; scale bar 50 μm) magnification. n=3 per treatment group; mock-infected animals (mock; n=2) received buffered saline instead of virus inoculum. BI, blood vessel; Br, bronchiole; arrow, interstitial pneumonia; asterisks, alveolitis.

FIG. 27. Schematic of the chemical synthesis strategy of the AVG scaffold. Reagents and conditions to generate analogs A and B from compound 1 with intermediates 2, 3, and 4 were (a) Dimethyl 1,3-acetonedicarboxylate, toluene, reflux, 12 hours, 90%; (b) MeC(OEt)₃, AcOH, CH3CN, 70° C., 12 hours; (c) (6-Chloropyridin-2-yl)methanamine dihydrochloride, DIPEA, CH3CN, 2 hours, DBU, 2-4 hours, 35%; and (d) substituted benzyl halide, DIPEA, 50° C., 2-3 hours, 40-50%.

FIG. 28. Schematic of the chemical synthesis strategy of AVG analog C. Reagents and conditions to generate analog C from compound 5 (prepared as shown in FIG. 27) were (a) NaN₃, NH₄Cl, DMF, 48 hours, 110° C., 36%.

The Following Tables are Provided which are Utilized or Referred to in Conjunction with this Example:

### Table 3 Shows the Efficacy of AVG Series In Vitro.

Dose response inhibition assays of recRSV-fireSMASh incubated with selected AVG-233 fluorine and chlorine analogs in a human cell line or primary human airway epithelium cells (CI, confidence interval; nd, not determined).

TABLE 3

| | Cell line or type | | | | | | | | | | | | in | |
|---|---|---|---|---|---|---|---|---|---|---|---|---|---|---|
| | HEp-2 | | | | “F1” HAE | | | | “M4” HAE | | | | vitro RdRP | |
| | C₅₀ | 5% | C₅₀ | 5% | C₅₀ | 5% | C₅₀ | 5% | C₅₀ | 5% | C₅₀ | 5% | C₅₀ | 5% |
| Compound | (μM) | I | (μM) | I | (μM) | I | (μM) | I | (μM) | I | (μM) | I | (μM) | I |
| AVG- | .24 | .21 to | 50 | 50 | .36 | .25 to | 45 | 45 | .11 | .06 to | 45 | 45 | .83 | .36 to |
| 233 | | 0.27 | | | | 0.52 | | | | 0.20 | | | | 14.58 |
| AVG- | .05 | .05 to | 10 | 10 | .13 | .09 to | 10 | 10 | .15 | .09 to | 10 | 10 | .25 | .15 to |
| 388 | | 0.06 | | | | 0.18 | | | | 0.25 | | | | 21568 |
| AVG- | .09 | .09 to | 5 | 5 | .16 | .12 to | 45 | 45 | .07 | .05 to | 45 | 45 | .88 | .60 to |
| 389 | | 0.10 | | | | 0.2149 | | | | 0.10 | | | | 6.60 |
| AVG- | .11 | .10 to | 45 | 45 | .39 | .27 to | 45 | 45 | .25 | .12 to | 45 | 45 | .62 | .69 to |
| 390 | | 0.13 | | | | 0.56 | | | | 0.57 | | | | 14.10 |
| AVG- | .96 | .79 to | 45 | 45 | .35 | .29 to | 45 | 45 | .30 | .21 to | 45 | 45 | 7.39 | 5.89 to |
| 391 | | 1.18 | | | | 5.01 | | | | 35.02 | | | | 19.04 |
| AVG- | .81 | .71 to | 45 | 45 | .92 | .64 to | 45 | 45 | .53 | .54 to | 45 | 45 | 3.11 | 5.30 to |
| 392 | | 0.93 | | | | 1.33 | | | | 8.83 | | | | 35.74 |
| AVG- | .2 | .15 to | 5 | 5 | d | d | d | d | d | d | d | d | .89 | .29 to |
| 435 | | 0.27 | | | | | | | | | | | | 3.20 |
| AVG- | .08 | .07 to | 5 | 5 | d | d | d | d | d | d | d | d | .63 | .12 to |
| 436 | | 0.11 | | | | | | | | | | | | 10.65 |
| AVG- | .2 | .15 to | 5 | 5 | d | d | d | d | d | d | d | d | .53 | .28 to |
| 445 | | 0.26 | | | | | | | | | | | | 37.21 |

Table 4 shows the comparison of AVG-233 and AVG-388 resistance profiles. Minireplicon activity^{a} and recRSV-fireSMASh^{b} activity in the presence or absence of resistance mutations in RSV L (CI, confidence interval) as described above.

TABLE 4

| Assay | Cell line | RSV L | AVG-233 EC₅₀ | 95% CI | AVG-388 EC₅₀ | 95% CI |
|---|---|---|---|---|---|---|
| minireplicon^{a} | HEK-293T | WT | 0.15 | 0.11 to 0.19 | 0.15 | 0.12 to 0.19 |
| minireplicon^{a} | HEK-293T | L1502Q | 24.04 | 18.46 to 30.95 | 89.45 | 61.84 to 161.4 |
| minireplicon^{a} | BSR T7/5 | WT | 0.19 | 0.13 to 0.32 | 0.31 | (—) |
| minireplicon^{a} | BSR T7/5 | L1502Q | 29.16 | 18.03 to 45.81 | 78.2 | 52.38 to 147.3 |
| minireplicon^{a} | BSR T7/5 | H1632Q | 4.57 | 2.85 to 7.17 | 6.24 | 1.87 to 33.15 |
| recombinant^{b} | HEp-2 | WT | 0.24 | 0.23 to 0.25 | 0.09 | 0.08 to 0.10 |
| recombinant^{b} | HEp-2 | L1502Q | >100.0 | >100.0 | >100.0 | >100.0 |
| recombinant^{b} | HEp-2 | Y1631H | 2.19 | (—) | 2.72 | 1.43 to 5.26 |
| recombinant^{b} | HEp-2 | H1632Q | 4.42 | (—) | 4.81 | (—) |

Table 5 shows the efficacy of AVG series in viva.

Lung viral load 4.5 days post-infection after therapeutic treatment (10 hours after infection).

TABLE 5

| | | Lung viral load reduction (log10 | |
|---|---|---|---|
| compound | dose | TCID₅₀/ml) relative to vehicle-treated | SD |
| AVG-233 | 50 mg/kg bid | −0.44 | 0.38 |
| AVG-388 | 50 mg/kg bid | −1.3 | 0.25 |
| AVG-388 | 150 mg/kg bid | −1.9 | 0.23 |
| AVG-389 | nd | nd | nd |
| AVG-390 | 50 mg/kg bid | −0.5 | 0.26 |
| AVG-391 | nd | nd | nd |
| AVG-392 | nd | nd | nd |
| AVG-435 | 50 mg/kg bid | −0.44 | 0.36 |
| AVG-436 | 50 mg/kg bid | −0.89 | 0.14 |
| AVG-445 | 50 mg/kg bid | −1.1 | 0.16 |

### REFERENCES

The following references were cited in the above example and are all incorporated by reference herein as if set forth in their entirety.
1. T. Shi et al., Global, regional, and national disease burden estimates of acute lower respiratory infections due to respiratory syncytial virus in young children in 2015: a systematic review and modelling study. Lancet 390, 946-958 (2017).
2. N. I. Mazur et al., The respiratory syncytial virus vaccine landscape: lessons from the graveyard and promising candidates. Lancet Infect. Dis. 18, e295-e311 (2018).
3. J. Gottlieb et al., A phase 2b randomized controlled trial of presatovir, an oral RSV fusion inhibitor, for the treatment of respiratory syncytial virus (RSV) in lung transplant (LT) recipients. The Journal of Heart and Lung Transplantation 37, S155 (2018).
4. D. Hanfelt-Goade et al., in C17. New insights in acute pulmonary infections. (American Thoracic Society, 2018), pp. A4457-A4457.
5. D. Yan et al., Cross-resistance mechanism of respiratory syncytial virus against structurally diverse entry inhibitors. Proc. Natl. Acad. Sci. U.S.A 111, E3441-3449 (2014).
6. T. I.-R. S. V. S. Group, Palivizumab, a humanized respiratory syncytial virus monoclonal antibody, reduces hospitalization from respiratory syncytial virus infection in high-risk infants. Pediatrics 102, 531-537 (1998).
7. T. F. Feltes et al., Palivizumab prophylaxis reduces hospitalization due to respiratory syncytial virus in young children with hemodynamically significant congenital heart disease. J. Pediatr. 143, 532-540 (2003).
8. M. Aggarwal, R. K. Plemper, Structural Insight into Paramyxovirus and Pneumovirus Entry Inhibition. Viruses 12, (2020).
9. R. Fearns, R. K. Plemper, Polymerases of paramyxoviruses and pneumoviruses. Virus Res. 234, 87-102 (2017).
10. H. Grosfeld, M. G. Hill, P. L. Collins, RNA replication by respiratory syncytial virus (RSV) is directed by the N, P, and L proteins; transcription also occurs under these conditions but requires RSV superinfection for efficient synthesis of full-length mRNA. J. Virol. 69, 5677-5686 (1995).
11. J. Sourimant et al., Fine mapping and characterization of the L-polymerase-binding domain of the respiratory syncytial virus phosphoprotein. J Virol 89, 4421-4433 (2015).
12. M. Galloux et al., Identification and characterization of the binding site of the respiratory syncytial virus phosphoprotein to RNA-free nucleoprotein. J Virol 89, 3484-3496 (2015).
13. R. Fearns, P. L. Collins, Role of the M2-1 transcription antitermination protein of respiratory syncytial virus in sequential transcription. J. Virol. 73, 5852-5864 (1999).
14. E. Burke, N. M. Mahoney, S. C. Almo, S. Barik, Profilin is required for optimal actin-dependent transcription of respiratory syncytial virus genome RNA. J Virol 74, 669-675 (2000).
15. A. P. Oliveira et al., Human respiratory syncytial virus N, P and M protein interactions in HEK-293T cells. Virus Res 177, 108-112 (2013).
16. C. A. Richard et al., RSV hijacks cellular protein phosphatase 1 to regulate M2-1 phosphorylation and viral transcription. PLoS Pathog 14, e1006920 (2018).
17. D. Cao et al., Cryo-EM structure of the respiratory syncytial virus RNA polymerase. Nat Commun 11, 368 (2020).
18. M. S. A. Gilman et al., Structure of the Respiratory Syncytial Virus Polymerase Complex. Cell 179, 193-204 e114 (2019).
19. J. Chapman et al., RSV604, a novel inhibitor of respiratory syncytial virus replication. Antimicrob. Agents Chemother. 51, 3346-3353 (2007).
20. B. Bailly et al., Targeting human respiratory syncytial virus transcription anti-termination factor M2-1 to inhibit in vivo viral replication. Sci. Rep. 6, 25806 (2016).
21. G. S. Cockerill, J. A. D. Good, N. Mathews, State of the Art in Respiratory Syncytial Virus Drug Discovery and Development. J. Med. Chem. 62, 3206-3227 (2019).
22. J. Sourimant et al., 4′-Fluorouridine is an oral antiviral that blocks respiratory syncytial virus and SARS-CoV-2 replication. Science, 375, 161-167 (2022).
23. C. L. Tiong-Yip et al., Characterization of a respiratory syncytial virus L protein inhibitor. Antimicrob. Agents Chemother. 58, 3867-3873 (2014).
24. R. M. Cox et al., Development of an allosteric inhibitor class blocking RNA elongation by the respiratory syncytial virus polymerase complex. J. Biol. Chem. 293, 16761-16777 (2018).
25. K. Sudo et al., YM-53403, a unique anti-respiratory syncytial virus agent with a novel mechanism of action. Antiviral Res. 65, 125-131 (2005).
26. M. Coates et al., Preclinical Characterization of PC786, an Inhaled Small-Molecule Respiratory Syncytial Virus L Protein Polymerase Inhibitor. Antimicrob. Agents Chemother. 61, (2017).
27. V. A. Laganas et al., Characterization of novel respiratory syncytial virus inhibitors identified by high throughput screen. Antiviral Res. 115, 71-74 (2015).
28. M. Dochow, S. A. Krumm, J. E. Crowe, Jr., M. L. Moore, R. K. Plemper, Independent structural domains in paramyxovirus polymerase protein. J. Biol. Chem. 287, 6878-6891 (2012).
29. O. Poch, I. Sauvaget, M. Delarue, N. Tordo, Identification of four conserved motifs among the RNA-dependent polymerase encoding elements. EMBO J. 8, 3867-3874 (1989).
30. J. Fix, M. Galloux, M. L. Blondot, J. F. Eleouet, The insertion of fluorescent proteins in a variable region of respiratory syncytial virus L polymerase results in fluorescent and functional enzymes but with reduced activities. Open Virol. J. 5, 103-108 (2011).
31. J. A. Horwitz, S. Jenni, S. C. Harrison, S. P. J. Whelan, Structure of a rabies virus polymerase complex from electron cryo-microscopy. Proc. Natl. Acad. Sci. U.S.A 117, 2099-2107 (2020).
32. S. Jenni et al., Structure of the Vesicular Stomatitis Virus L Protein in Complex with Its Phosphoprotein Cofactor. Cell reports 30, 53-60 e55 (2020).
33. B. Liang et al., Structure of the L Protein of Vesicular Stomatitis Virus from Electron Cryomicroscopy. Cell 162, 314-327 (2015).
34. R. J. Pickles, Human airway epithelial cell cultures for modeling respiratory syncytial virus infection. Curr Top Microbiol Immunol 372, 371-387 (2013).
35. M. Toots et al., Characterization of orally efficacious influenza drug with high resistance barrier in ferrets and human airway epithelia. Sci. Transl. Med. 11, (2019).
36. L. Zhang, M. E. Peeples, R. C. Boucher, P. L. Collins, R. J. Pickles, Respiratory syncytial virus infection of human airway epithelial cells is polarized, specific to ciliated cells, and without obvious cytopathology. J. Virol. 76, 5654-5666 (2002).
37. V. Rincheval et al., Functional organization of cytoplasmic inclusion bodies in cells infected by respiratory syncytial virus. Nat Commun 8, 563 (2017).
38. J. J. Yoon et al., Orally Efficacious Broad-Spectrum Ribonucleoside Analog Inhibitor of Influenza and Respiratory Syncytial Viruses. Antimicrob. Agents Chemother. 62, (2018).
39. U.S. Food & Drug Administration. DA News Release Coronavirus (COVID-19) Update: FDA Authorizes Additional Oral Antiviral for Treatment of COVID-19 in Certain Adults. https://www.fda.gov/news-events/press-announcements/coronavirus-covid-19-update-fda-authorizes-additional-oral-antiviral-treatment-covid-19-certain. (2021).
40. D. B. Madhura, J. Liu, B. Meibohm, R. E. Lee, Phase II Metabolic Pathways of Spectinamide Antitubercular Agents: A Comparative Study of the Reactivity of 4-Substituted Pyridines to Glutathione Conjugation. Medchemcomm 7, 114-117 (2016).
41. J. Litchfield et al., Intrinsic electrophilicity of the 4-methylsulfonyl-2-pyridone scaffold in glucokinase activators: role of glutathione-S-transferases and in vivo quantitation of a glutathione conjugate in rats. Bioorg. Med. Chem. Lett. 20, 6262-6267 (2010).
42. K. Inoue et al., Aromatic substitution reaction of 2-chloropyridines catalyzed by microsomal glutathione S-transferase 1. Drug Metab. Dispos. 37, 1797-1800 (2009).
43. J. R. Duvall et al., Novel diversity-oriented synthesis-derived respiratory syncytial virus inhibitors identified via a high throughput replicon-based screen. Antiviral Res. 131, 19-25 (2016).
44. S. L. Noton et al., Respiratory Syncytial Virus Inhibitor AZ-27 Differentially Inhibits Different Polymerase Activities at the Promoter. J. Virol. 89, 7786-7798 (2015).
45. R. M. Cox, J. Sourimant, M. Govindarajan, M. G. Natchus, R. K. Plemper, Therapeutic targeting of measles virus polymerase with ERDRP-0519 suppresses all RNA synthesis activity. PLoS Pathog. 17, e1009371 (2021).
46. P. C. Jordan et al., Initiation, extension, and termination of RNA synthesis by a paramyxovirus polymerase. PLoS Pathog. 14, e1006889 (2018).
47. J. Pan et al., Structure of the human metapneumovirus polymerase phosphoprotein complex. Nature 577, 275-279 (2020).
48. R. Abdella, M. Aggarwal, T. Okura, R. A. Lamb, Y. He, Structure of a paramyxovirus polymerase complex reveals a unique methyltransferase-CTD conformation. Proc. Natl. Acad. Sci. U.S.A 117, 4931-4941 (2020).
49. M. Ogino, N. Gupta, T. J. Green, T. Ogino, A dual-functional priming-capping loop of rhabdoviral RNA polymerases directs terminal de novo initiation and capping intermediate formation. Nucleic Acids Res. 47, 299-309 (2019).
50. M. R. Braun et al., RNA elongation by respiratory syncytial virus polymerase is calibrated by conserved region V. PLoS Pathog. 13, e1006803 (2017).
51. A. L. Hotard et al., A stabilized respiratory syncytial virus reverse genetics system amenable to recombination-mediated mutagenesis. Virology 434, 129-136 (2012).
52. D. Yan et al., Replication-Competent Influenza Virus and Respiratory Syncytial Virus Luciferase Reporter Strains Engineered for Co-Infections Identify Antiviral Compounds in Combination Screens. Biochemistry 54, 5589-5604 (2015).
53. B. Ludeke, R. Fearns, The respiratory syncytial virus polymerase can perform RNA synthesis with modified primers and nucleotide analogs. Virology 540, 66-74 (2020).
54. S. L. Noton, L. R. Deflube, C. Z. Tremaglio, R. Fearns, The respiratory syncytial virus polymerase has multiple RNA synthesis activities at the promoter. PLoS Pathog. 8, e1002980 (2012).
55. J. Schindelin et al., Fiji: an open-source platform for biological-image analysis. Nat. Methods 9, 676-682 (2012).
56. J. Deval et al., Molecular Basis for the Selective Inhibition of Respiratory Syncytial Virus RNA Polymerase by 2′-Fluoro-4′-Chloromethyl-Cytidine Triphosphate. PLoS Pathog. 11, e1004995 (2015).
57. E. P. Tchesnokov, P. Raeisimakiani, M. Ngure, D. Marchant, M. Gotte, Recombinant RNA-Dependent RNA Polymerase Complex of Ebola Virus. Sci Rep 8, 3970 (2018).
58. H. Chi et al., Comprehensive identification of peptides in tandem mass spectra using an efficient open search engine. Nat. Biotechnol., (2018).
59. J. Fix, M. Galloux, M. L. Blondot, J. F. Eleouet, The insertion of fluorescent proteins in a variable region of respiratory syncytial virus L polymerase results in fluorescent and functional enzymes but with reduced activities. Open Virol. J. 5, 103-108 (2011).
60. S. A. Jablonski, C. D. Morrow, Mutation of the aspartic acid residues of the GDD sequence motif of poliovirus RNA-dependent RNA polymerase results in enzymes with altered metal ion requirements for activity. J. Virol. 69, 1532-1539 (1995).

What is claimed is:

1. A method of inhibiting a respiratory infection or disease comprising administering to a patient in need thereof, an effective amount of a compound of Formula 1a: 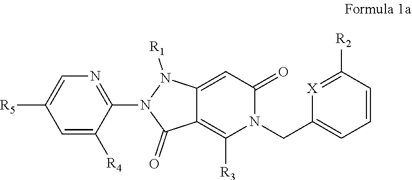
or a pharmaceutically acceptable salt thereof, wherein
X is N, C, —NR⁰, or —CR^{a}R^{b};
R⁰ and R¹ are independently selected from the group consisting of —R^{c}, —SO₂R^{c}, —SO₂N(R^{c})₂; —CR^{c}, —C(O)R^{c}, —COOR^{c}, or —C(O)N(R^{c})₂,
wherein R^{c} is selected from the group consisting of hydrogen, C₁₋₈ alkyl, C₃₋₈ cycloalkyl, C₂₋₈ heterocyclyl, C₆₋₁₂ aryl, C₃₋₁₂ heteroaryl, C₁₋₈ alkyl-C₃₋₈ cycloalkyl, C₁₋₈ alkyl-C₂₋₈ heterocyclyl, C₁₋₈ alkyl-C₆₋₁₂ aryl, and C₁₋₈ alkyl-C₃₋₁₂ heteroaryl; and wherein when R^{c} is not hydrogen, R^{c} may be optionally substituted at any position;
R², R³, R⁴, R⁵, R^{a}, and R^{b} are independently selected from the group consisting of R^{c}, —OR^{c}, —N(R^{c})₂, —SR^{c}, —SO₂R^{c}, —SO₂N(R^{c})₂; —CR^{c}, —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —CF₃, or —NO₂;
wherein two or more of R⁰, R¹, R², R³, R⁴, R⁵, R^{a} and R^{b} can together form a ring;
wherein any two of the aforementioned R groups when adjacent can together form a double bond;
wherein two of the aforementioned R groups, when germinal, can together form a carbonyl, olefin or imine; and
wherein one or more of R^{c} can together form a ring with any one or more of R⁰, R¹, R², R³, R⁴, R⁵, R^{a}, R^{b} and R^{c}.

2. The method according to claim 1 wherein the respiratory infection or disease is caused by a virus selected from the group consisting of RSV, coronavirus, SARS-CoV-2, SARS, pneumovirus, paramyxovirus, metapneumovirus, mumps virus, human parainfluenzaviruses, Nipah virus (NIV), and hendra virus.

3. The method according to claim 1, wherein X is N.

4. The method according to claim 1, wherein R¹ is optionally substituted C₁₋₈ alkyl-C₆₋₁₂ aryl.

5. The method according to claim 1, wherein R¹ is an optionally substituted benzyl.

6. The method according to claim 1, wherein R² is —CF₃, or —Cl.

7. The method according to claim 1, wherein R³ is C₁₋₈ alkyl.

8. The method according to claim 1, wherein R³ is methyl.

9. The method according to claim 1, wherein R⁴ and R⁴ are independently H or F.

10. The method according to claim 1, wherein R¹ is selected from the group consisting of:

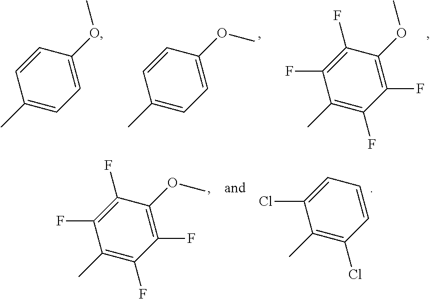

11. A method of inhibiting a respiratory infection or disease comprising administering to a patient in need thereof, an effective amount of a compound of Formula 1 b: 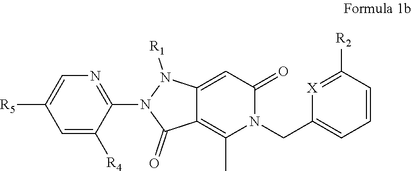
or a pharmaceutically acceptable salt thereof, wherein
X is N, C, —NR⁰, or —CR^{a}R^{b};
R⁰ and R¹ are independently selected from the group consisting of —R^{c}, —SO₂R^{c}, —SO₂N(R^{c})₂; —CR^{c}, —C(O)R^{c}, —COOR^{c}, substituted or unsubstituted benzyl, and —C(O)N(R^{c})₂,
wherein R^{c} is selected from the group consisting of hydrogen, C₁₋₈ alkyl, C₃₋₈ cycloalkyl, C₂₋₈ heterocyclyl, C₆₋₁₂ aryl, C₃₋₁₂ heteroaryl, C₁₋₈ alkyl-C₃₋₈ cycloalkyl, C₁₋₈ alkyl-C₂₋₈ heterocyclyl, C₁₋₈ alkyl-C₆₋₁₂ aryl, and C₁₋₈ alkyl-C₃₋₁₂ heteroaryl; and wherein when R^{c} is not hydrogen, R^{c} may be optionally substituted at any position;
R², R⁴, R⁵, R^{a}, and R^{b} are independently selected from the group consisting of R^{c}, —OR^{c}, —N(R^{c})₂, —SR^{c}, —SO₂R^{c}, —SO₂N(R^{c})₂; —CR^{c}, —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —CF₃, or —NO₂;
wherein two or more of R⁰, R¹, R², R⁴, R⁵, R^{a} and R^{b} can together form a ring;
wherein any two of the aforementioned R groups when adjacent can together form a double bond;
wherein two of the aforementioned R groups, when germinal, can together form a carbonyl, olefin or imine; and
wherein one or more of R^{c} can together form a ring with any one or more of R⁰, R¹, R², R⁴, R⁵, R^{a}, R^{b} and R^{c}.

12. The method according to claim 11, wherein X is N.

13. The method according to claim 11, wherein R¹ is optionally substituted C₁₋₈ alkyl-C₆₋₁₂ aryl.

14. The method according to claim 11, wherein R¹ is an optionally substituted benzyl.

15. The method according to claim 1, wherein R² is —CF₃, or —Cl.

16. A method of inhibiting a respiratory infection or disease comprising administering to a patient in need thereof, an effective amount of a compound of Formula 1c: 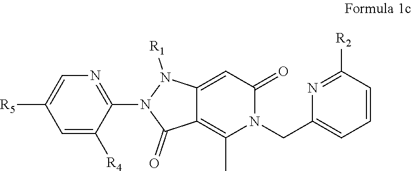
or a pharmaceutically acceptable salt thereof, wherein
R⁰ and R¹ are independently selected from the group consisting of —R^{c}, —SO₂R^{c}, —SO₂N(R^{c})₂; —CR^{c}, —C(O)R^{c}, —COOR^{c}, substituted or unsubstituted benzyl, and —C(O)N(R^{C})₂,
wherein R^{c} is selected from the group consisting of hydrogen, C₁₋₈ alkyl, C₃₋₈ cycloalkyl, C₂₋₈ heterocyclyl, C₆₋₁₂ aryl, C₃₋₁₂ heteroaryl, C₁₋₈ alkyl-C₃₋₈ cycloalkyl, C₁₋₈ alkyl-C₂₋₈ heterocyclyl, C₁₋₈ alkyl-C₆₋₁₂ aryl, and C₁₋₈ alkyl-C₃₋₁₂ heteroaryl; and wherein when R^{c} is not hydrogen, R^{c} may be optionally substituted at any position;
R², R⁴, R⁵, R^{a}, and R^{b} are independently selected from the group consisting of R^{c}, —OR^{c}, —N(R^{c})₂, —SR^{c}, —SO₂R^{c}, —SO₂N(R^{c})₂; —CR^{c}, —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —CF₃, or —NO₂;
wherein two or more of R⁰, R¹, R², R⁴, R⁵, R^{a} and R^{b} can together form a ring;
wherein any two of the aforementioned R groups when adjacent can together form a double bond;
wherein two of the aforementioned R groups, when germinal, can together form a carbonyl, olefin or imine; and
wherein one or more of R^{c} can together form a ring with any one or more of R⁰, R¹, R², R⁴, R⁵, R^{a}, R^{b} and R^{c}.

17. A method of treating or preventing a respiratory infection, comprising administering to a patient in need thereof an effective amount of a compound of Formula 1a: 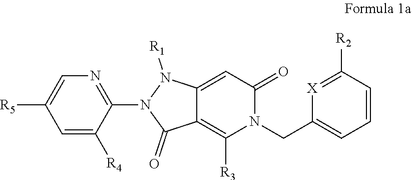
or a pharmaceutically acceptable salt thereof, wherein
X is N, C, —NR⁰, or —CR^{a}R^{b};
R⁰ and R¹ are independently selected from the group consisting of —R^{c}, —SO₂R^{c}, —SO₂N(R^{c})₂; —CR^{c}, —C(O)R^{c}, —COOR^{c}, or —C(O)N(R^{c})₂,
wherein R^{c} is selected from the group consisting of hydrogen, C₁₋₈ alkyl, C₃₋₈ cycloalkyl, C₂₋₈ heterocyclyl, C₆₋₁₂ aryl, C₃₋₁₂ heteroaryl, C₁₋₈ alkyl-C₃₋₈ cycloalkyl, C₁₋₈ alkyl-C₂₋₈ heterocyclyl, C₁₋₈ alkyl-C₆₋₁₂ aryl, and C₁₋₈ alkyl-C₃₋₁₂ heteroaryl; and wherein when R^{c} is not hydrogen, R^{c} may be optionally substituted at any position;
R², R³, R⁴, R⁵, R^{a}, and R^{b} are independently selected from the group consisting of R^{c}, —OR^{c}, —N(R^{c})₂, —SR^{c}, —SO₂R^{c}, —SO₂N(R^{c})₂; —CR^{c}, —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —CF₃, or —NO₂;
wherein two or more of R⁰, R¹, R², R³, R⁴, R⁵, R^{a} and R^{b} can together form a ring;
wherein any two of the aforementioned R groups when adjacent can together form a double bond;
wherein two of the aforementioned R groups, when germinal, can together form a carbonyl, olefin or imine; and
wherein one or more of R^{c} can together form a ring with any one or more of R⁰, R¹, R², R³, R⁴, R⁵, R^{a}, R^{b} and R^{c}

18. A method of inhibiting RSV comprising administering to a patient in need thereof, an effective amount of a compound of Formula 1a: 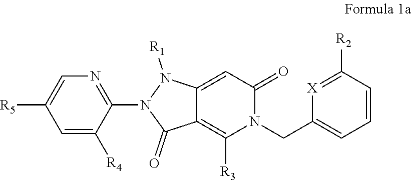
or a pharmaceutically acceptable salt thereof, wherein
X is N, C, —NR⁰, or —CR^{a}R^{b};
R⁰ and R¹ are independently selected from the group consisting of —R^{c}, —SO₂R^{c}, —SO₂N(R^{c})₂; —CR^{c}, —C(O)R^{c}, —COOR^{c}, or —C(O)N(R^{c})₂,
wherein R^{c} is selected from the group consisting of hydrogen, C₁₋₈ alkyl, C₃₋₈ cycloalkyl, C₂₋₈ heterocyclyl, C₆₋₁₂ aryl, C₃₋₁₂ heteroaryl, C₁₋₈ alkyl-C₃₋₈ cycloalkyl, C₁₋₈ alkyl-C₂₋₈ heterocyclyl, C₁₋₈ alkyl-C₆₋₁₂ aryl, and C₁₋₈ alkyl-C₃₋₁₂ heteroaryl; and wherein when R^{c} is not hydrogen, R^{c} may be optionally substituted at any position;
R², R³, R⁴, R⁵, R^{a}, and R^{b} are independently selected from the group consisting of R^{c}, —OR^{c}, —N(R^{c})₂, —SR^{c}, —SO₂R^{c}, —SO₂N(R^{c})₂; —CR^{c}, —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —CF₃, or —NO₂;
wherein two or more of R⁰, R¹, R², R³, R⁴, R⁵, R^{a} and R^{b} can together form a ring;
wherein any two of the aforementioned R groups when adjacent can together form a double bond;
wherein two of the aforementioned R groups, when germinal, can together form a carbonyl, olefin or imine; and
wherein one or more of R^{c} can together form a ring with any one or more of R⁰, R¹, R², R³, R⁴, R⁵, R^{a}, R^{b} and R^{c}.

19. A method of treating or preventing an RSV infection, comprising administering to a patient in need thereof an effective amount of a compound of Formula 1a: 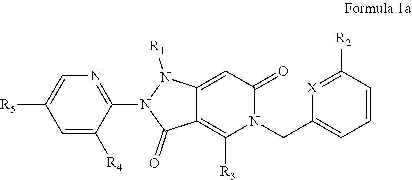
or a pharmaceutically acceptable salt thereof, wherein
X is N, C, —NR⁰, or —CR^{a}R^{b};
R⁰ and R¹ are independently selected from the group consisting of —R^{c}, —SO₂R^{c}, —SO₂N(R^{c})₂; —CR^{c}, —C(O)R^{c}, —COOR^{c}, or —C(O)N(R^{c})₂,
wherein R^{c} is selected from the group consisting of hydrogen, C₁₋₈ alkyl, C₃₋₈ cycloalkyl, C₂₋₈ heterocyclyl, C₆₋₁₂ aryl, C₃₋₁₂ heteroaryl, C₁₋₈ alkyl-C₃₋₈ cycloalkyl, C₁₋₈ alkyl-C₂₋₈ heterocyclyl, C₁₋₈ alkyl-C₆₋₁₂ aryl, and C₁₋₈ alkyl-C₃₋₁₂ heteroaryl; and wherein when R^{c} is not hydrogen, R^{c} may be optionally substituted at any position;
R², R³, R⁴, R⁵, R^{a}, and R^{b} are independently selected from the group consisting of R^{c}, —OR^{c}, —N(R^{c})₂, —SR^{c}, —SO₂R^{c}, —SO₂N(R^{c})₂; —CR^{c}, —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —CF₃, or —NO₂;
wherein two or more of R⁰, R¹, R², R³, R⁴, R⁵, R^{a} and R^{b} can together form a ring;
wherein any two of the aforementioned R groups when adjacent can together form a double bond;
wherein two of the aforementioned R groups, when germinal, can together form a carbonyl, olefin or imine; and
wherein one or more of R^{c} can together form a ring with any one or more of R⁰, R¹, R², R³, R⁴, R⁵, R^{a}, R^{b} and R^{c}

20. The method of treating or preventing an RSV infection according to claim 19 wherein the compound is administered via a route of administration selected from the group consisting of buccal, oral, intravenous, inhalation, intradermal, intramuscular, topical, subcutaneous, rectal, vaginal, parenteral, pulmonary, intranasal, and ophthalmic.

21. A compound of Formula 1a: 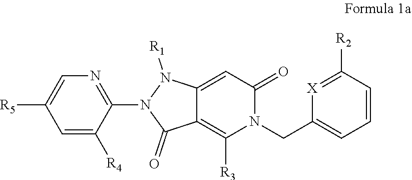
or a pharmaceutically acceptable salt thereof, wherein
X is N, C, —NR⁰, or —CR^{a}R^{b};
R⁰ and R¹ are independently selected from the group consisting of —R^{c}, —SO₂R^{c}, —SO₂N(R^{c})₂; —CR^{c}, —C(O)R^{c}, —COOR^{c}, or —C(O)N(R^{c})₂,
wherein R^{c} is selected from the group consisting of hydrogen, C₁₋₈ alkyl, C₃₋₈ cycloalkyl, C₂₋₈ heterocyclyl, C₆₋₁₂ aryl, C₃₋₁₂ heteroaryl, C₁₋₈ alkyl-C₃₋₈ cycloalkyl, C₁₋₈ alkyl-C₂₋₈ heterocyclyl, C₁₋₈ alkyl-C₆₋₁₂ aryl, and C₁₋₈ alkyl-C₃₋₁₂ heteroaryl; and wherein when R^{c} is not hydrogen, R^{c} may be optionally substituted at any position;
R², R³, R⁴, R⁵, R^{a}, and R^{b} are independently selected from the group consisting of R^{c}, —OR^{c}, —N(R^{c})₂, —SR^{c}, —SO₂R^{c}, —SO₂N(R^{c})₂; —CR^{c}, —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —CF₃, or —NO₂;
wherein two or more of R⁰, R¹, R², R³, R⁴, R⁵, R^{a} and R^{b} can together form a ring;
wherein any two of the aforementioned R groups when adjacent can together form a double bond;
wherein two of the aforementioned R groups, when germinal, can together form a carbonyl, olefin or imine; and
wherein one or more of R^{c} can together form a ring with any one or more of R⁰, R¹, R², R³, R⁴, R⁵, R^{a}, R^{b} and R^{c}.

22. The compound according to claim 21 wherein the compound is selected from the group consisting of:

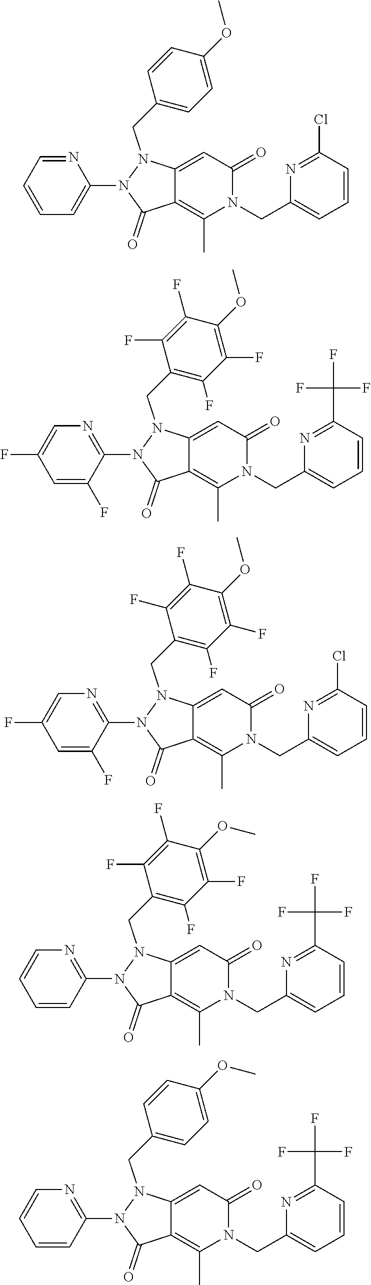

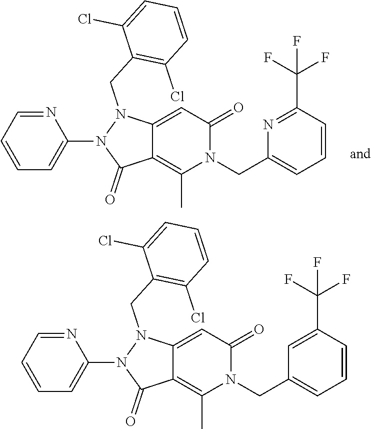

23. A pharmaceutical composition comprising the compound of claim 21 and a pharmaceutically acceptable carrier, vehicle, or excipient.

24. A pharmaceutical composition comprising a compound of Formula 1a: 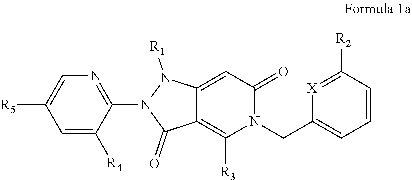
or a pharmaceutically acceptable salt thereof, wherein
X is N, C, —NR⁰, or —CR^{a}R^{b};
R⁰ and R¹ are independently selected from the group consisting of —R^{c}, —SO₂R^{c}, —SO₂N(R^{c})₂; —CR^{c}, —C(O)R^{c}, —COOR^{c}, or —C(O)N(R^{c})₂,
wherein R^{c} is selected from the group consisting of hydrogen, C₁₋₈ alkyl, C₃₋₈ cycloalkyl, C₂₋₈ heterocyclyl, C₆₋₁₂ aryl, C₃₋₁₂ heteroaryl, C₁₋₈ alkyl-C₃₋₈ cycloalkyl, C₁₋₈ alkyl-C₂₋₈ heterocyclyl, C₁₋₈ alkyl-C₆₋₁₂ aryl, and C₁₋₈ alkyl-C₃₋₁₂ heteroaryl; and wherein when R^{c} is not hydrogen, R^{c} may be optionally substituted at any position;
R², R³, R⁴, R⁵, R^{a}, and R^{b} are independently selected from the group consisting of R^{c}, —OR^{c}, —N(R^{c})₂, —SR^{c}, —SO₂R^{c}, —SO₂N(R^{c})₂; —CR^{c}, —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —CF₃, or —NO₂;
wherein two or more of R⁰, R¹, R², R³, R⁴, R⁵, R^{a} and R^{b} can together form a ring;
wherein any two of the aforementioned R groups when adjacent can together form a double bond;
wherein two of the aforementioned R groups, when germinal, can together form a carbonyl, olefin or imine; and
wherein one or more of R^{c} can together form a ring with any one or more of R⁰, R¹, R², R³, R⁴, R⁵, R^{a}, R^{b} and R^{c};
and a pharmaceutically acceptable carrier, vehicle, or excipient.

25. The pharmaceutical composition according to claim 24 wherein the compound is selected from the group consisting of:

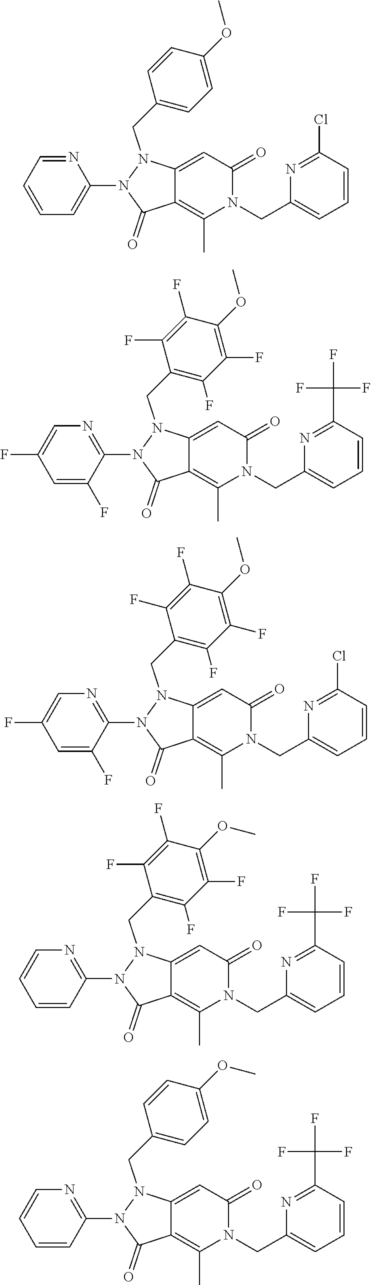

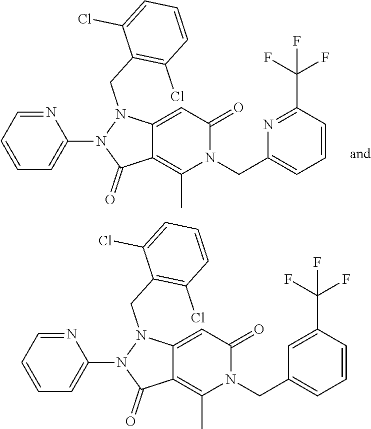

26. A method of inhibiting or impairing RNA elongation of viral RNA in a virus that causes a respiratory infection, disease, illness, or other respiratory condition, said method comprising administering to a patient in need thereof an effective amount of a compound of Formula 1a.

27. The method according to claim 26 wherein the virus is selected from the group consisting of RSV, coronavirus, SARS-CoV-2, SARS, pneumovirus, paramyxovirus, metapneumovirus, mumps virus, human parainfluenzaviruses, Nipah virus (NIV), and hendra virus.

28. The method according to claim 26 wherein the virus is RSV.

29. A method of blocking viral RNA-dependent RNA polymerase of a virus that causes a respiratory infection, disease, or other respiratory condition, said method comprising administering to a patient in need thereof an effective amount of a compound of Formula 1a.

30. The method according to claim 29 wherein the virus is selected from the group consisting of RSV, coronavirus, SARS-CoV-2, SARS, pneumovirus, paramyxovirus, metapneumovirus, mumps virus, human parainfluenzaviruses, Nipah virus (NIV), and hendra virus.

31. The method according to claim 29 wherein the virus is RSV.

32. The method according to claim 29 wherein the blocking is non-competitive blocking.
